(19) United States
(12) Patent Application Publication
(10) Pub. No.: US 2023/0270575 A1
(43) Pub. Date: Aug. 31, 2023

(54) DEVICES AND METHODS FOR GASTRIC RETENTIVE DRUG DELIVERY

(71) Applicant: Teva Pharmaceutical Industries Ltd., Tel Aviv (IL)

(72) Inventors: Avshalom BEN MENACHEM, Tel Aviv (IL); Ilan ZALIT, Tel Aviv (IL)

(21) Appl. No.: 18/023,706

(22) PCT Filed: Aug. 27, 2021
(86) PCT No.: PCT/IB2021/057886

§ 371 Date: Feb. 27, 2023

(30) Foreign Application Priority Data
Aug. 30, 2020 (EP) 20193509.5

(51) Int. Cl. A61F 5/00

(52) U.S. Cl. CPC A61F 5/0036

(57) ABSTRACT

The present disclosure provides with a device for temporary residence in a stomach of a subject, the device carrying a load of an active pharmaceutical ingredient and having an expanded configuration. When the device is positioned in the stomach and in the expanded configuration, the device is configured to be retained in the stomach of the subject, and for releasing at least partially the active pharmaceutical ingredient while allowing chyme flow. The device is further configured to transfer from the expanded configuration into an emptying configuration in which the device is configured to pass through the pyloric valve. This enables dispensing locally therapeutic substances at a gastric region of a human body.

### CROSS REFERENCE TO RELATED APPLICATION

This application claims the benefit of European patent application no. 20193509.5, filed Aug. 30, 2020, the entirety of which is incorporated by reference herein.

### TECHNOLOGICAL FIELD

The present disclosure relates generally to the field of regio-specific release of a substance at a site of a gastrointestinal (GI) tract. More specifically, the present disclosure relates to devices and methods for dispensing locally therapeutic substances at a gastric region of a human body (gastric retentive drug delivery).

### BACKGROUND

The gastrointestinal (GI) tract generally provides a therapeutic medium for an individual's body. At times, therapeutic drugs may need to be dispensed during a specified continuous period of time within the stomach to cure or alleviate the symptoms of some medical conditions. However, locally dispensing therapeutic drugs for a sufficiently long period of time, i.e. for example within the stomach inside a human body, can be difficult and requires a device or mechanism (e.g., special platform) to stay or to be retained in the stomach for sufficient time and release the therapeutic drug for a specified period of time. Such a device or mechanism also would need to be operated in a safe manner, in that the device or mechanism needs to physically enter and empty of the human body.

Dispensing therapeutic drugs within the stomach inside a human body for a sufficiently long period of time may be useful for enabling treatment of a disease, for example by releasing a medication directly in the vicinity of the stomach (e.g., when this is a disease site) and/or for improving action of medications which have an improved absorption into the blood circulation.

Gastric retentive drug delivery systems and gastric retentive drug delivery dosage forms (GRDF) would be particularly useful for the delivery of drugs that are preferentially absorbed in the stomach, and/or have better solubility in stomach, and/or intended for local treatment of stomach and/or duodenum, and/or poorly soluble in alkaline pH or intestine, and/or those drugs that degrade in the colon or in the intestine, and/or those drugs that disturb colonic microbes, and/or those drugs with poor patient adherence, or drugs that have slow and or incomplete intestinal absorption, short biological half-life and therapeutic index is low, e.g., absorption is limited to upper small intestine (narrow absorption window) such as the duodenum and or jejunum.

A desired GRDF would be orally dosed, be retained in the stomach for a prolonged predictable period of time, during which it would release the drug in a predefined manner, while after releasing a sufficient amount of drug, the device will (e.g., disintegrate or disassemble and) be emptied out of the stomach and eventually emptied out of the body. When operating in body, the device would be safe. The device would have to operate in a consistent way under variable human GI physiology (intra-inter patient variability in GI motility, GI forces, GI dimension, stomach pylorus size, change in fed state during the day, etc.). In practice, however, there are many challenges in designing GRDF systems. Examples include ability to manufacture and scale up, use of biocompatible materials, sufficient drug loading capacity, small swallow size while having controlled retention capabilities in the stomach during fed and fasted states. Further challenges are safety while being resident in stomach for long periods, and including methods to control the emptying time from the stomach to optimize treatment as well as emergency expelling, etc., (e.g., see Seong Hoon Jeong et al, “Current State and Future Perspectives on Gastroretentive Drug Delivery Systems”).

Improvements in any of these challenges would provide a significant contribution in the field of GRDF to enable new and improved treatment opportunities for the patient. Improvements may include:
1. Topical treatment: improved treatment regimens for gastrointestinal diseases, including a need for regimens which can dispense therapeutics in the gastric region for prolonged time like the stomach, the pyloric region and the proximal duodenum, thereby reducing or avoiding the drawbacks of other forms of administration.
2. Improved systemic therapeutic exposure: achieving systemic plasma concentrations having minimal fluctuations enabling better efficacy and safety.

Improvements in any of these directions would provide a significant contribution to the area of gastric retentive drug delivery systems and dosage forms.

### GENERAL DESCRIPTION

The present disclosure notably provides methods and devices for specifically releasing a therapeutic substance in the gastric region, which alleviate at least in part the limitations of the prior art techniques.

Gastric release has great benefits for some therapeutic agents when used in various medical conditions:
a. Treatment of a gastric ulcer, or gastroesophageal reflux disease.
b. Treatment of stomach cancer or stomach infections.
c. Improved systemic therapeutic exposure by durable absorption and improved bioavailability, i.e., achieving systemic plasma concentrations having minimal fluctuations enabling better efficacy and safety.

Therefore, the present disclosure provides devices, an oral dosage form, and a method, according to any of the following clauses:
1. A device for temporary residence in a stomach of a subject, the device carrying a load of an active pharmaceutical ingredient and having an expanded configuration, wherein when the device is in the expanded configuration, the device:
   (a) is configured to be retained in the stomach of the subject;
   (b) is configured for releasing at least partially the active pharmaceutical ingredient; and
   the device being further configured to transfer from the expanded configuration into an emptying configuration in which the device is configured to pass through the pyloric valve.
2. The device according to clause 1, wherein the device is configured for allowing chyme flow therethrough when the device is positioned in the stomach, for example near the pylorus valve, and in the expanded configuration.
3. The device according to clause 1 or 2, wherein the device further has a closed configuration and is deformable from the closed configuration into the expanded configuration. Optionally, the device may be reversibly deformable from the expanded configuration into the closed configuration, for example during manufacturing.
4. The device according to clause 3, wherein the dimensions of the device in the closed configuration are such that it can be ingested and preferably fitted in a cylinder of length of about 35 mm or less and/or of diameter of about 12 mm or less, preferably in a cylinder of length equal to or less than 32 mm, 31 mm, 30 mm or 29 mm and/or of diameter equal to or less than 11, 10.5 mm or 10.2 mm, preferably a cylinder of length equal to or less that 31 mm and/or of diameter equal to or less than 11 mm, more preferably in a cylinder of length equal to 29 mm and of diameter equal to 10.2 mm.
5. The device according to any of clauses 3 to 4, wherein the device in the closed configuration has a compact shape.
6. The device according to any of clauses 3 to 5, further comprising an opening assembly configured to transfer the device from the closed configuration into the expanded configuration.
7. The device according to clause 6, wherein the opening assembly comprises a biasing assembly configured to bias the device in the expanded configuration so that it tends to resiliently return from the closed configuration into the expanded configuration.
8. The device according to clause 7, wherein:
   (a) the device comprises:
   at least two flexible arms, each arm having a first end and a second end;
   (b) the biasing assembly comprises:
      i) a first and second coupling heads, wherein the first end of each flexible arm is coupled to the first coupling head and the second end of each flexible arm is coupled to the second coupling head;
      ii) a resiliently deformable member arranged between the at least two arms and configured to force the coupling heads together to bend the arms thereby biasing the device in the expanded configuration (e.g., wherein the coupling heads may come close one to another in the trapping configuration thanks to retraction of the resiliently deformable member, i.e., the resiliently deformable member forces the coupling heads to transit/move towards each other during transfer/deformation/transit of the device from the closed configuration into the expanded configuration, such that the coupling heads are closer one to another in the expanded configuration than in the closed configuration).
9. The device according to clause 8, wherein the device is configured to transfer from the expanded configuration into the emptying configuration upon the resiliently deformable member stopping to force the coupling heads together.
10. The device according to any of clauses 8 to 9, wherein the resiliently deformable member is arranged between the arms.
11. The device according to any of clauses 8 to 10, wherein each arm is arranged longitudinally alongside each other.
12. The device according to any of clauses 8 to 11, wherein the device comprises at least three or at least four such flexible arms, the resiliently deformable member being optionally arranged between the at least three or at least four arms.
13. The device according to any of clauses 8 to 12, wherein the device further comprises one or more circumferential belts circling around the flexible arms, and/or one or more circumferential threads circumferentially linking the flexible arms.
14. The device according to clause 12, wherein the device further comprises one or more circumferential belts circling around the at least three or at least four flexible arms, and/or one or more circumferential threads circumferentially linking the at least three or at least four flexible arms.
15. The device according to any of clauses 8 to 14, further comprising a container to temporarily at least partially enclose the device in the closed configuration, wherein the container is configured to degrade in stomach environmental conditions.
16. The device according to any of clauses 8 to 15, wherein the resiliently deformable member is releasably secured to at least one of the first and second coupling heads.
17. The device according to clause 16, wherein the resiliently deformable member is configured to be released from the at least one of the first and second coupling heads when the device in the expanded configuration is exposed to a predetermined activation signal.
18. The device according to any of clauses 8 to 17, wherein the first end of each flexible arm is coupled to the first coupling head releasably, and the second end of each flexible arm is coupled to the second coupling head releasably.
19. The device according to clause 18, wherein, in the expanded configuration, said first and second ends are each inserted in a respective cavity of the first and second coupling heads, the resiliently deformable member maintaining said first and second end each secured inside the respective cavity.
20. The device according to clause 19, wherein said first and second ends are each rotatable in the respective cavity.
21. The device according to any of clauses 19 to 20, wherein, when the device transfers from the expanded configuration into the emptying configuration, said first and second ends are each dimensioned to move out of the respective cavity (i.e., the dimensioning enables such moving out), the device being thereby configured for the disassembling of the flexible arms from the first and second coupling heads. In particular, the detachment of the arms from the head may be possible when the device is in the emptying configuration, and still the detachment of the arms from the head cavities may be induced by GI motility. For example, the transfer into the emptying configuration may move the arm ends out of abutment with abutment surfaces in the cavities, thereby enabling the ends to move out of the cavities.
22. The device according to clause 21, wherein the resiliently deformable member is releasably secured to at least one of the first and second coupling heads, and the resiliently deformable member is configured to be released from the at least one of the first and second coupling heads when the device in the expanded configuration is exposed to a predetermined activation signal, thereby causing the device to transfer from the expanded configuration into the emptying configuration.
23. The device according to any of clauses 8 to 22, wherein the device further comprises a support tube, the resiliently deformable member being arranged inside the support tube.
24. The device according to clause 23, the device carries at least part of the load of the active pharmaceutical ingredient inside or on the support tube, for example in an interstice formed between the support tube and the resiliently deformable member.
25. The device according to clause 24, wherein the at least part of the load of the active pharmaceutical ingredient is carried inside or on the support tube in a solid, semi-solid, powder, gel, and/or liquid form.
26. The device according to any of clauses 23 to 25, wherein the support tube has apertures formed thereon, the apertures providing exposure the inside of the support tube.
27. The device according to any of clauses 23 to 26, wherein the support tube has one or more peripheral grooves each lodging a ring-shaped form containing the active pharmaceutical ingredient.
28. The device according to any of clauses 8 to 27, wherein at least one (e.g., each) flexible arm is articulated and/or composed of a set of arm sections, wherein said arm sections (e.g., of each arm) are preferably rigid.
29. The device according to clause 28, wherein at least one (e.g., each) arm section comprises one or more integrally formed components.
30. The device according to clause 29, wherein the one or more integrally formed components of said at least one arm section comprise one or more integrally formed components made of a rigid material, one or more integrally formed components made of a semi-rigid material, and/or one or more integrally formed components made of a flexible material.
31. The device according to any of clauses 29 to 30, wherein each integrally formed component of said at least one arm section is 3D printed or injection molded.
32. The device according to any of clauses 28 to 31, wherein at least two (e.g., all) arm sections of at least one (e.g., each) flexible arm are coupled end to end with a pivot-type coupling.
33. The device according to clause 32, wherein the pivot-type coupling comprises transversal hinge holes of the arm sections of the at least two arm sections and a hinge connector passing through the transversal hinge holes.
34. The device according to any of clauses 28 to 33, wherein each flexible arm consists of two arm sections, each arm section being coupled at one end to the first or second coupling head and at the other end to the other arm section.
35. The device according to clause 34, wherein each arm section is substantially of a same length, the device having a generally bipyramidal shape in the expanded configuration, preferably a generally octahedral shape.
36. The device according to any of clauses 28 to 35, wherein at least one arm section comprises an exposed cavity.
37. The device according to clause 36, wherein the at least one arm section comprises two components attached one to another and forming the exposed cavity there between.
38. The device according to clause 37, wherein the two components are snapped one to another.
39. The device according to any of clauses 36 to 38, wherein the at least one arm section comprises a peripheral wall, the peripheral wall having apertures formed thereon, the apertures providing exposure of the exposed cavity.
40. The device according to any of clauses 36 to 39, wherein the exposed cavity contains at least part of the load of the active pharmaceutical ingredient.
41. The device according to clause 40, wherein the at least part of the load of the active pharmaceutical ingredient is contained in the exposed cavity in a solid, semi-solid, powder, gel, and/or liquid form.
42. The device according to any of clauses 8 to 27, wherein each flexible arm is made of an elastic material, the resiliently deformable member being configured to bend the arms in a rounded shape.
43. The device according to clause 42, wherein each flexible arm comprises an integrally formed component made of the elastic material, the resiliently deformable member being configured to bend the integrally formed component in a rounded shape.
44. The device according to any of clauses 42 to 43, wherein the elastic material is silicone.
45. The device according to any of clauses 8 to 44, wherein the resiliently deformable member is elastic.
46. The device according to clause 45, wherein the resiliently deformable member is made of silicone.
47. The device according to any of clauses 8 to 44, wherein the resiliently deformable member is a spring.
48. The device according to clause 47, wherein the spring is made of metal, preferably stainless steel or Nitinol.
49. The device according to any of clauses 8 to 48, wherein the arms are at least partially made of a material that gradually erodes in the intestine, so as to facilitate emptying out of the body.
50. The device according to any of clauses 3 to 49, further comprising a locking element arranged to temporary maintain the device in the closed configuration for facilitating administration.
51. The device according to clause 50, wherein the locking element is configured to release the device in the expanded configuration in the stomach when the device is positioned in the stomach.
52. The device according to any of clauses 50 to 51, wherein the locking element is at least partially made of a material degrading at standard stomach environmental conditions.
53. The device according to any of clauses 50 to 52, wherein the locking element is a container and the device is contained in the container in the closed configuration.
54. The device according to any of clauses 50 to 52, wherein the device comprises, additionally to the locking element, a container and the device is contained in the container in the closed configuration.
55. The device according to any of clauses 1 to 54, wherein the device in the expanded configuration is configured to be capable of moving in the stomach by standard stomach motility without damaging the stomach.
56. The device according to clause 55, wherein the dimensions of the device in the expanded configuration are such that it can be fitted in a sphere of a diameter of about 35 mm or less, and preferably of about 30 mm or 25 mm or less.
57. The device according to any of the preceding clauses, wherein the device comprises an inner space in the closed configuration formed between components of the device, the inner space containing at least part of the load of the active pharmaceutical ingredient.
58. The device according to 57, wherein the device comprises at least one portion or component which comprises an exposed cavity, the exposed cavity containing at least part of the load of the active pharmaceutical ingredient.
59. The device according to clause 58, wherein the at least one portion or component comprises a peripheral wall, the peripheral wall having apertures formed thereon, the apertures providing exposure of the exposed cavity.
60. The device according to any of the preceding clauses, wherein the device comprises an exposed recess, the exposed recess lodging at least part of the load of the active pharmaceutical ingredient.
61. The device according to any of the preceding clauses, wherein the device comprises a coating on an exposed surface, the coating containing at least part of the load of the active pharmaceutical ingredient.
62. The device according to any of the preceding clauses, wherein the load of the active pharmaceutical ingredient is carried in a solid, semi-solid, powder, gel, and/or liquid form.
63. The device according to any of clauses 57 to 62, wherein the load of the active pharmaceutical ingredient occupies at least 5% or at least 10% or at least 15% or at least 20% or preferably at least 25% of the volume of a convex hull of the device in the closed configuration.
64. The device according to any of the preceding clauses, wherein each coupling head (of clause 8) comprises one or more rigid and/or integrally formed components, each coupling head preferably being rigid and integrally formed.
65. The device according to any of the preceding clauses, wherein an external envelope of the device in the expanded configuration is configured to contact the subject tissue and the external envelope of the device is configured to avoid damaging the subject tissue.
66. The device according to clause 65, wherein the external envelope of the device is flexible and/or blunt to avoid damaging the subject tissue.
67. The device according to any of the preceding clauses, wherein the device further comprises an external padding structure.
68. The device according to any of the preceding clauses, wherein a volume occupied by the device in the expanded configuration is larger than a sphere of a diameter of about 17 mm, and optionally larger than a sphere of a diameter of about 20 mm. In other words, an external hull of the device in the expanded configuration sticks out of a sphere of a diameter of about 17 mm and optionally of a sphere of a diameter of about 20 mm.
69. The device according to any of the preceding clauses, wherein the device comprises a meshed structure having one or more openings configured for allowing chyme flow through the device, when the device is in the expanded configuration.
70. The device according to any of the preceding clauses, further comprising a trigger assembly configured to cause the device to transfer from the expanded configuration into the emptying configuration upon activation.
71. The device according to clause 70, wherein the trigger assembly is configured to activate when the device in the expanded configuration is exposed to an activation signal.
72. The device according to clause 71, wherein the activation signal comprises exposure to standard stomach environmental conditions for a predetermined residence time period, wherein preferably the predetermined residence time period one day, two days, three days or more and/or twelve weeks or less, for example one month, one week, two weeks, three weeks, one day, two days or three days (i.e., the expanded device is designed to be retained in the stomach for said residence time period before being transferred into the emptying configuration).
73. The device according to any of clauses 71 to 72, wherein the activation signal comprises exposure of the device in the expanded configuration to at least one activation environmental condition.
74. The device according to clause 73, wherein the activation environmental condition comprises a surrounding environment reaching a predetermined pH threshold, for example reaching above pH 6. 75. The device according to any of clauses 71 to 74, wherein the activation signal comprises an electromagnetic or magnetic or ultrasound signal.
76. The device according to any of clauses 70 to 75, wherein the trigger assembly comprises one or more support elements configured to temporarily maintain the device in the expanded configuration.
77. The device according to clause 76, wherein the one or more support elements are configured to be disabled upon activation of the trigger assembly thereby causing the device to transfer into the emptying configuration.
78. The device according to any of clauses 76 to 77, wherein the one or more support elements are configured to be disabled when the device in the expanded configuration is exposed to the activation signal.
79. The device according to any of clauses 76 to 78, wherein the one or more support elements are configured to be disabled by degradation when the device in the expanded configuration is exposed to the activation signal.
80. The device according to any of clauses 76 to 79, wherein the one or more support elements are configured to be disabled when a surrounding pH reaches a predetermined pH threshold.
81. The device according to clause 80, wherein the one or more support elements are at least partially made of a pH dependent polymer configured to dissolve when the surrounding pH reaches the predetermined pH threshold, preferably when the surrounding pH reaches above pH 6.
82. The device according to any of clauses 76 to 79, wherein the one or more support elements are configured to be disabled when a predetermined time period elapsed subsequent to positioning of the device in the stomach.
83. The device according to clause 82, wherein the one or more support elements are at least partially made of a material configured to degrade at standard stomach environmental conditions.
84. The device according to clause 83, wherein the one or more support elements comprise a material configured to degrade when the surrounding pH is between 1 and 3.
85. The device according to any of clauses 76 to 84, wherein the device comprises one or more first support elements according to clause 61 and additionally one or more second support elements according to clause 64. In some embodiments, the one or more second support elements are configured to degrade faster than the one or more first support elements.
86. The device according to any of clauses 76 to 84, wherein at least one (e.g., each) of the one or more support elements is an integrally formed component presenting a diameter (i.e., length of longest straight segment from a point of object to another point of object) shorter than a half (preferably shorter than a fourth) of a diameter of the device in the expanded configuration. In examples where the device is configured to transfer from the expanded configuration into the emptying configuration by disassembling into at least two disassembled subcomponents, such support element may present a diameter shorter than a half (preferably shorter than a fourth) of a diameter of each disassembled subcomponent (e.g., at the time when the disassembling occurs in case the disassembled subcomponent is not rigid). Such a support element may enable localized degradation until emptying of the device, thus little affecting (at least not substantially) structural rigidity, shape, and/or size of the device while in the expanded configuration. The integrally formed component may be separate or distinct from at least part (e.g., all) of the carried/embedded load of API.
87. The device according to any of clauses 76 to 86, wherein the one or more support elements are configured to form one or more structural weak points of the device to enable a collapse of the device into the emptying configuration after the device is exposed to the activation signal. This may contribute to providing for a rapid transfer from the expanded configuration into the emptying configuration. This also participates in enabling the device to maintain structural integrity until the device transfers into the emptying configuration thereby improving device efficiency until collapse. Furthermore, the components of the device which play a significant role in the main functionalities of the device such as stomach retention and/or API release may generally be configured to not be affected by the activation signal. For example, these components (e.g., arms and/or biasing assembly) may not be made of a material configured to degrade at (i.e., may be made of a material configured to resist to) the environmental conditions of the stomach in embodiments in which the activation signal is exposure to standard stomach region environmental conditions for a predetermined residence time period.
87. The device according to any of clauses 76 to 86, wherein the one or more support elements are configured to be disabled in less than 7 days, 3 days, 1 day or ½ day after said the device is exposed to the activation signal.
89. The device according to any of clauses 70 to 88, wherein the trigger assembly is configured such that the device in the expanded configuration maintains structural integrity (i.e., any one or more of shape, size and/or structural rigidity) until transfer in the emptying configuration is effected (i.e., achieved).
90. The device according to any of the preceding clauses, wherein the device is configured to transfer from the expanded configuration into the emptying configuration by changing shape.
91. The device according to any of the preceding clauses, wherein the device is configured to transfer from the expanded configuration into the emptying configuration by changing size.
92. The device according to any of the preceding clauses, wherein the device is configured to transfer from the expanded configuration into the emptying configuration by decrease of structural rigidity.
93. The device according to any of the preceding clauses, wherein the device is configured to transfer from the expanded configuration into the emptying configuration by disassembling into at least two disassembled subcomponents.
94. The device according to any of clauses 1 to 93, wherein the device in the closed configuration (of clause 3) or emptying configuration can pass through the pyloric valve of the subject when positioned in the stomach.
95. The device according to clause 94, wherein an external envelope of the device in the emptying configuration and an external envelope of the device in the closed configuration are of same dimensions.
96. The device according to any of clauses 94 to 95, wherein the closed configuration of the device is the same as the emptying configuration of the device.
97. The device according to any of the preceding clauses, wherein the trigger assembly (of clause 70) and the opening assembly (of clause 6) form a single transferring assembly configured to transfer the device from the closed configuration (of clause 3) into the expanded configuration and to transfer the device from the expanded configuration into the emptying configuration upon activation.
98. The device according to any of the preceding clauses, wherein the one or more support elements (of clause 76) are configured to cooperate with the biasing assembly (of clause 7) so that, when the one or more support elements are disabled, the biasing of the biasing assembly is prevented and the device thereby transfers into the emptying configuration.
99. The device according to any of the preceding clauses, wherein in the expanded configuration, any one or more of a shape of the device and/or a size of the device and/or a structural rigidity of the device prevent passage of the device through the pyloric valve, when the device is positioned in the stomach.
100. The device according to any of the preceding clauses, further comprising a labelling element enabling detection of the device in a subject by external imaging means, wherein the imaging means is optionally X-ray imaging.
101. The device according to any of the preceding clauses, wherein the device has in the expanded configuration a convex hull presenting a (e.g., Hakon Wadell) sphericity above 0.8, and/or a ratio between a maximal (e.g., planar) circumference and a minimal (e.g., planar) circumference below 1.5.
102. A device for temporary residence in a stomach of a subject, the device comprising:
   at least two flexible arms, each arm having a first end and a second end;
   a first and second coupling heads, wherein the first end of each arm is coupled to the first coupling head and the second end of each arm is coupled to the second coupling head;
   a resiliently deformable member configured to force the coupling heads together to bend the arms thereby biasing the device in an expanded configuration (e.g., wherein the coupling heads may come close one to another in the trapping configuration thanks to retraction of the resiliently deformable member, i.e., the resiliently deformable member forces the coupling heads to transit/move towards each other during transfer/deformation/transit of the device from the closed configuration into the expanded configuration, such that the coupling heads are closer one to another in the expanded configuration than in the closed configuration), wherein in the expanded configuration, the device is configured, when the device is positioned in the stomach, to be retained in the stomach of the subject; and
the device being further configured to transfer from the expanded configuration into an emptying configuration, the device being configured to pass through the pyloric in the emptying configuration;
the device further carrying a load of an active pharmaceutical ingredient and being configured, when the device is in the expanded configuration and positioned in the stomach, for releasing at least partially the active pharmaceutical ingredient.
103. The device according to clause 102, wherein the device is configured for allowing chyme flow therethrough, when the device is positioned in the stomach and in the expanded configuration.
104. The device according to any of clauses 102 to 103, wherein the at least two flexible arms comprise at least three or at least four such flexible arms (i.e., the device comprises at least three or at least four flexible arms as recited in clause 102).
105. The device according to any of clauses 102 to 104, wherein each arm is arranged longitudinally alongside each other.
106. The device according to any of clauses 102 to 105, wherein the device is configured to transfer from the expanded configuration into the emptying configuration upon the resiliently deformable member stopping to force the coupling heads together.
107. The device according to any of clauses 102 to 106, wherein the resiliently deformable member is arranged between the arms.
108. The device according to any of clauses 102 to 107, wherein the device further comprises one or more circumferential belts circling around the arms, and/or one or more circumferential threads circumferentially linking the arms.
109. The device according to any of clauses 102 to 108, the device has a closed configuration, and in the closed configuration, the resiliently deformable member is stretched and the arms are substantially straight. In the expanded configuration, the arms are bent, and the resiliently deformable member may be stretched, but less that in the closed configuration. In other words, an extension of the resiliently deformable member may be larger in the closed configuration than in the expanded configuration and the arms may be substantially straight in the closed configuration. Optionally, in the manufacturing process, the resiliently deformable member may be reversibly stretched, thereby straightening the at least three arms to reversibly deform the device from the expanded configuration into the closed configuration. Optionally, in the manufacturing process, the resiliently deformable member may be reversibly stretched, thereby straightening the at least three arms to reversibly deform the device from the expanded configuration into the closed configuration.
110. The device according to clause 109, wherein the dimensions of the device in the closed configuration are such that it can be ingested and preferably fitted in a cylinder of length of about 35 mm or less and/or of diameter of about 12 mm or less, preferably in a cylinder of length equal to or less than 32 mm, 31 mm, 30 mm or 29 mm and/or of diameter equal to or less than 11, 10.5 mm or 10.2 mm, preferably a cylinder of length equal to or less that 31 mm and/or of diameter equal to or less than 11 mm, more preferably in a cylinder of length equal to 29 mm and of diameter equal to 10.2 mm.
111. The device according to any of clauses 109 to 110, further comprising a container to temporarily at least partially enclose the device in the closed configuration, wherein the container is configured to degrade in stomach environmental conditions.
112. The device according to any of clauses 109 to 111, further comprising a locking element arranged to temporary maintain the device in the closed configuration for facilitating administration.
113. The device according to clause 112, wherein the locking element is configured to release the device in the expanded configuration in the stomach when the device is positioned in the stomach.
114. The device according to any of clauses 112 to 113, wherein the locking element is at least partially made of a material degrading at standard stomach environmental conditions.
115. The device according to any of clauses 112 to 114, wherein the locking element is a container and the device is contained in the container in the closed configuration.
116. The device according to any of clauses 112 to 114, wherein the device comprises, additionally to the locking element, a container and the device is contained in the container in the closed configuration.
117. The device according to any of clauses 102 to 106, wherein the first end of each arm is coupled to the first coupling head releasably, and the second end of each arm is coupled to the second coupling head releasably.
118. The device according to clause 117, wherein, in the expanded configuration, said first and second ends are each inserted in a respective cavity of the first and second coupling heads, the resiliently deformable member maintaining said first and second end each secured inside the respective cavity.
119. The device according to clause 118, wherein said first and second ends are each rotatable in the respective cavity.
120. The device according to any of clauses 118 to 119, wherein, when the device transfers from the expanded configuration into the emptying configuration, said first and second ends are each dimensioned to move out of the respective cavity (i.e., the dimensioning enables such moving out), the device being thereby configured for the disassembling of the arms from the first and second coupling heads. In particular, the detachment of the arms from the head may be possible when the device is in the emptying configuration, and still the detachment of the arms from the head cavities may be induced by GI motility. For example, the transfer into the emptying configuration may move the arm ends out of abutment with abutment surfaces in the cavities, thereby enabling the ends to move out of the cavities.
121. The device according to any of clauses 102 to 120, wherein the resiliently deformable member is secured to both the first and second coupling heads, and the resiliently deformable member is configured, for each respective activation signal from a predetermined set of one or more activation signals, to be released from at least one respective coupling head from the first and second coupling heads, when the device in the expanded configuration is exposed to the respective activation signal, thereby causing the device to transfer from the expanded configuration into the emptying configuration.
122. The device according to clause 121, further comprising a trigger assembly configured, when the device in the expanded configuration is exposed to the respective activation signal, to release the resiliently deformable member from the at least one respective coupling head, thereby causing the device to transfer from the expanded configuration into the emptying configuration.
123. The device according to clause 122, wherein the predetermined set of one or more activation signals includes a signal which comprises exposure to standard stomach environmental conditions for a predetermined residence time period, wherein preferably the predetermined residence time period is one day, two days, three days or more and/or twelve weeks or less, for example one month, two weeks, three weeks, one day, two days, one week, or three days (i.e., the expanded device is designed to be retained in the stomach for said residence time period before being transferred into the emptying configuration).
124. The device according to any of clauses 122 to 123, wherein the predetermined set of one or more activation signals includes a signal which comprises exposure of the device in the expanded configuration to at least one activation environmental condition.
125. The device according to clause 124, wherein the signal which comprises exposure of the device in the expanded configuration to at least one activation environmental condition comprises a surrounding environment reaching a predetermined pH threshold.
126. The device according to any of clauses 122 to 125, wherein the predetermined set of one or more activation signals includes a signal which comprises an electromagnetic or magnetic or ultrasound signal.
127. The device according to any of clauses 122 to 126, wherein the trigger assembly comprises one or more support elements configured to temporarily secure the resiliently deformable member to the at least one respective coupling head, and thereby temporarily maintain the device in the expanded configuration.
128. The device according to clause 127, wherein, for each respective activation signal from the predetermined set of one or more activation signals, at least one respective support element from the one or more support elements is configured to be disabled upon the device in the expanded configuration being exposed to the respective activation signal, thereby causing release of the resiliently deformable member from the at least one of the first and second coupling heads, thus further causing the device to transfer into the emptying configuration.
129. The device according to clause 128, wherein the at least one respective support element is configured to be disabled by degradation when the device in the expanded configuration is exposed to the respective activation signal.
130. The device according to clause 129, wherein, before degradation, the at least one respective support element securely attaches the at least one respective coupling head to the resiliently deformable member, and upon degradation, the resiliently deformable member retracts and detaches from the at least one respective coupling head.
131. The device according to any of clauses 127 to 130, wherein the one or more support elements comprise at least one given support element configured to be disabled when a surrounding pH reaches a predetermined pH threshold, thus causing the device to transfer into the emptying configuration.
132. The device according to clause 131, wherein the at least one given support element, configured to be disabled when a surrounding pH reaches a predetermined pH threshold, is at least partially made of a pH dependent polymer configured to dissolve when the surrounding pH reaches the predetermined pH threshold, preferably when the surrounding pH reaches above pH 6.
133. The device according to any of clauses 127 to 132, wherein the one or more support elements comprise at least one given support element configured to be disabled when a predetermined time period elapsed subsequent to positioning of the device in the stomach, thus causing the device to transfer into the emptying configuration.
134. The device according to clause 133, wherein the at least one given support element, configured to be disabled when a predetermined time period elapsed subsequent to positioning of the device in the stomach, is at least partially made of a material configured to degrade at standard stomach environmental conditions.
135. The device according to clause 134, wherein the at least one given support element, configured to be disabled when a predetermined time period elapsed subsequent to positioning of the device in the stomach, comprises a material configured to degrade when the surrounding pH is between 1 and 3.
136. The device according to any of clauses 127 to 135, wherein at least one (e.g., each) of the one or more support elements is an integrally formed component presenting a diameter (i.e., length of longest straight segment from a point of object to another point of object) shorter than a half (preferably shorter than a fourth) of a diameter of the device in the expanded configuration. In examples where the device is configured to transfer from the expanded configuration into the emptying configuration by disassembling into at least two disassembled subcomponents, such support element may present a diameter shorter than a half (preferably shorter than a fourth) of a diameter of each disassembled subcomponent (e.g., at the time when the disassembling occurs in case the disassembled subcomponent is not rigid). Such a support element may enable localized degradation until emptying of the device, thus little affecting (at least not substantially) structural rigidity, shape, and/or size of the device while in the expanded configuration. The integrally formed component may be separate or distinct from at least part (e.g., all) of the carried/embedded load of API.
137. The device according to any of clauses 127 to 136, wherein the one or more support elements are configured to form one or more structural weak points of the device to enable a collapse of the device into the emptying configuration after the device is exposed to the activation signal. The one or more structural weak points may enable (preferably sudden) detachment/release of the resiliently deformable member from the at least one respective coupling head. This may contribute to improving a (preferably rapid) transfer from the expanded configuration into the emptying configuration. This also participates in enabling the device to maintain structural integrity until the device transfers into the emptying configuration thereby enabling device efficiency until collapse. Furthermore, the components of the device which play a significant role in the main functionalities of the device such as stomach retention and/or API release may generally be configured to not be affected by the predetermined set of one or more activation signals. For example, these components (e.g., arms and/or biasing assembly) may not be made of a material configured to degrade at (i.e., may be made of a material configured to resist to) the environmental conditions of the stomach in embodiments in which the activation signal is exposure to standard stomach environmental conditions for a predetermined residence time period.
138. The device according to any of clauses 122 to 137, wherein the trigger assembly is configured such that the device in the expanded configuration maintains structural integrity (i.e., any one or more of shape, size and/or structural rigidity) until transfer in the emptying configuration is effected.
139. The device according to any of clauses 127 to 138, wherein the one or more support elements comprise at least one given support element configured to be disabled in less than 7 days, 3 days, 1 day or ½ day after said the device is exposed to the activation signal (preferably said activation comprising the exposure to stomach environmental conditions).
140. The device according to any of clauses 127 to 139, wherein the one or more support elements comprise several support elements each configured to be disabled upon the device in the expanded configuration being exposed to a different activation signal, thus causing the device to transfer into the emptying configuration.
141. The device according to any of clauses 127 to 140, wherein the one or more support elements comprise at least one given support element configured to form a structural weak point of the device to enable a collapse of the device into the emptying configuration after the device is exposed to a given activation signal (as in clause 137).
142. The device according to any of clauses 102 to 141, wherein the device further comprises a support tube, the resiliently deformable member being arranged inside the support tube.
143. The device according to clause 142, the device further carries at least part of the load of the active pharmaceutical ingredient inside or on the support tube, for example in an interstice formed between the support tube and the resiliently deformable member.
144. The device according to clause 143, wherein the at least part of the load of the active pharmaceutical ingredient is carried inside or on the support tube in a solid, semi-solid, powder, gel, and/or liquid form.
145. The device according to any of clauses 143 to 144, wherein the support tube has apertures formed thereon, the apertures providing exposure of the inside of the support tube.
146. The device according to any of clauses 142 to 145, wherein the support tube has one or more peripheral grooves each lodging a ring-shaped form containing the active pharmaceutical ingredient.
147. The device according to any of clauses 102 to 146, wherein at least one (e.g., each) arm is articulated and/or composed of a set of arm sections, wherein said arm sections (e.g., of each arm) are preferably rigid.
148. The device according to clause 147, wherein each arm section comprises one or more integrally formed components.
149. The device according to clause 148, wherein the one or more integrally formed components of each arm section comprise one or more integrally formed components made of a rigid material, one or more integrally formed components made of a semi-rigid material, and/or one or more integrally formed components made of a flexible material.
150. The device according to any of clauses 148 to 149, wherein each integrally formed component is 3D printed or injection molded.
151. The device according to any of clauses 147 to 150, wherein at least two (e.g., all) arm sections of at least one (e.g., each) arm are coupled end to end with a pivot-type coupling (e.g., said at least one arm being articulated at each pivot-type coupling).
152. The device according to clause 151, wherein the pivot-type coupling comprises transversal hinge holes of the arm sections of the at least two arm sections and a hinge connector passing through the transversal hinge holes.
153. The device according to any of clauses 147 to 152, wherein each arm consists of two arm sections, each arm section being coupled at one end to the first or second coupling head and at the other end to the other arm section.
154. The device according to clause 153, wherein each arm section is substantially of a same length, the device having a generally bipyramidal shape in the expanded configuration, preferably a generally octahedral shape.
155. The device according to any of clauses 147 to 154, wherein at least one arm section comprises an exposed cavity.
156. The device according to clause 155, wherein the at least one arm section comprises two components attached one to another and forming the exposed cavity therebetween.
157. The device according to clause 156, wherein the two arm section components are snapped one to another.
158. The device according to any of clauses 155 to 157, wherein the at least one arm section comprises a peripheral wall, the peripheral wall having apertures formed thereon, the apertures providing exposure of the exposed cavity.
159. The device according to any of clauses 155 to 158, wherein the exposed cavity contains at least part of the load of the active pharmaceutical ingredient.
160. The device according to clause 159, wherein the at least part of the load of the active pharmaceutical ingredient is contained in the exposed cavity in a solid, semi-solid, powder, gel, and/or liquid form.
161. The device according to any of clauses 102 to 146, wherein each flexible arm is made of an elastic material, the resiliently deformable member being configured to bend the arms in a rounded shape.
162. The device according to clause 161, wherein each flexible arm comprises an integrally formed component made of the elastic material, the resiliently deformable member being configured to bend the integrally formed component in a rounded shape.
163. The device according to any of clauses 161 to 162, wherein the elastic material is silicone.
164. The device according to any of clauses 102 to clause 163, wherein the resiliently deformable member is elastic.
165. The device according to clause 164, wherein the resiliently deformable member is made of silicone.
166. The device according to any of clauses 102 to 163, wherein the resiliently deformable member is a spring.
167. The device according to clause 166, wherein the spring is made of metal.
168. The device according to clause 167, wherein the metal is stainless steel or Nitinol.
169. The device according to any of clauses 102 to 168, wherein the arms are at least partially made of a material that gradually erodes in the intestine, so as to facilitate emptying out of the body.
170. The device according to any of clauses 102 to 169, wherein the device in the expanded configuration is configured to be capable of transiting in the stomach by standard GI motility without damaging the stomach.
171. The device according to clause 170, wherein the dimensions of the device in the expanded configuration are such that it can be fitted in a sphere of a diameter of about 35 mm or less, and preferably of about 30 mm or 25 mm or less.
172. The device according to any of clauses 102 to 171, wherein the device comprises an inner space in the closed configuration formed around the resiliently deformable member between the arms and the coupling heads, the inner space containing at least part of the load of the active pharmaceutical ingredient.
173. The device according to any of clauses 102 to 172, wherein the device comprises at least one portion or component which comprises an exposed cavity, the exposed cavity containing at least part of the load of the active pharmaceutical ingredient.
174. The device according to clause 173, wherein the at least one portion or component comprises a peripheral wall, the peripheral wall having apertures formed thereon, the apertures providing exposure of the exposed cavity.
175. The device according to any of clauses 102 to 174, wherein the device comprises an exposed recess, the exposed recess lodging at least part of the load of the active pharmaceutical ingredient.
176. The device according to any of clauses 102 to 175, wherein the device comprises a coating on an exposed surface, the coating containing at least part of the load of the active pharmaceutical ingredient.
177. The device according to any of clauses 102 to 176, wherein the load of the active pharmaceutical ingredient is carried in a solid, semi-solid, powder, gel, and/or liquid form.
178. The device according to any of clauses 102 to 177, wherein the load of the active pharmaceutical ingredient occupies at least 5% or at least 10% or at least 15% or at least 20% or preferably at least 25% of the volume of a convex hull of the device in the closed configuration.
179. The device according to any of clauses 102 to 178, wherein each coupling head comprises one or more rigid and/or integrally formed components, each coupling head preferably being rigid and integrally formed.
180. The device according to any of clauses 102 to 179, wherein an external envelope of the device in the expanded configuration is configured to contact the subject tissue and the external envelope of the device is configured to avoid damaging the subject tissue.
181. The device according to clause 180, wherein the external envelope of the device is blunt to avoid damaging the subject tissue.
182. The device according to any of clauses 102 to 181, wherein the device further comprises an external padding structure.
183. The device according to any of clauses 102 to 182, wherein a volume occupied by the device in the expanded configuration is larger than a sphere of a diameter of about 17 mm, and optionally larger than a sphere of a diameter of about 20 mm. In other words, an external hull of the device in the expanded configuration may stick out of a sphere of a diameter of about 17 mm, and optionally a sphere of a diameter of about 20 mm.
184. The device according to any of clauses 102 to 183, wherein the device comprises a meshed structure having one or more openings configured for allowing chyme flow through the device, when the device is in the expanded configuration.
185. The device according to any of clauses 102 to 184, wherein the device in the closed configuration (of clause 109) can pass through the pyloric valve of the subject when positioned in the stomach.
186. The device according to any clauses 102 to 185, wherein in the expanded configuration, any one or more of a shape of the device and/or a size of the device and/or a structural rigidity of the device prevent passage of the device through the pyloric valve, when the device is positioned in the stomach.
187. The device according to any of clauses 102 to 186, further comprising a labelling element enabling detection of the device in a subject by external imaging means, wherein the imaging means is optionally X-ray imaging.
188. The device according to any of clauses 102 to 187, wherein the device has in the expanded configuration a convex hull presenting a sphericity above 0.8, and/or a ratio between a maximal circumference and a minimal circumference below 1.5.
189. An oral dosage form for administering to a subject, the oral dosage form being intended for use in conjunction with a device according to any of clauses 1 to 101 (as dependent on clause 70) or according to any of clauses 102 to 188 (as dependent on clause 122) when said device is positioned in the stomach of the subject in the expanded configuration, wherein the dosage form comprises an effective amount of an emptying agent, wherein the amount of the emptying agent is sufficient to cause the stomach environment to reach at least one activation environmental condition thereby causing the device to transfer into the emptying configuration, such as the at least one activation environmental condition of clause 73 or of clause 124.
190. The oral dosage form according to clause 189, wherein the emptying agent comprises an amount of a base (antacid) sufficient to bring a stomach environmental pH above a pH threshold, for example pH 6.
191. The oral dosage form according to any of clauses 189 to 190, wherein the dosage form is a tablet having an oval or elliptical or capsule shape having a length of about 10 mm to 30 mm and a diameter of about 7 mm to 12 mm.
192. The oral dosage form according to any of clauses 189 to 191, wherein the base comprises aluminum hydroxide, magnesium carbonate, calcium carbonate sodium bicarbonate or such.
193. The oral dosage form according to any of clauses 190 to 192, wherein the pH threshold is 6.0 or 7.0.
194. The oral dosage form according to any of clauses 189 to 193, wherein the dosage is a gel form, a solid form, a semi-solid form, a liquid form, or any combination thereof.
195. A method of treating a condition which benefits from local dispensing of an active pharmaceutical ingredient (API) in the stomach of a subject suffering from said condition, comprising administering to said subject the device according to any of clauses 1 to 101 or the device according to any of clauses 102 to 188, thereby treating said condition in said subject.
196. The method according to clause 195, wherein the condition is a gastric disease, such as ulcer or cancer.
197. The method according to any of clauses 195 to 196, wherein the API released in the stomach is absorbed in the small intestine so as to treat said condition or improve treatment of said condition.
198. The method according to any of clauses 195 to 197, wherein the API is a drug that has at least one of the following: slow and or incomplete intestinal absorption, short biological half-life and therapeutic index (e.g., narrow absorption window), and/or high degradation in intestine.
199. The method according to any of clauses 195 to 198, comprising subsequently administering the oral dosage form according to any of clauses 189 to 193, thereby emptying the device.
200. The method according to clause 199, wherein the subsequently administering of the oral dosage form is performed a predetermined time after administering the device, the device being retained in the stomach during the predetermined time.
201. The method according to clause 200, wherein the predetermined time is at least 6 hours, at least 12 hours, at least 24 hours, at least 48 hours, at least three days, at least one week, at least two weeks, at least three weeks, or at least one month.
202. The method according to any of clauses 195 to 201, comprising subsequently administering one or more subsequent devices according to need or at constant intervals.
203. The method according to any of clauses 195 to 202, wherein the device has in the expanded configuration a convex hull presenting a sphericity above 0.8, and/or a ratio between a maximal circumference and a minimal circumference below 1.5.

The present disclosure also provides a kit comprising any device as previously disclosed, and optionally one or more conjunct emptying oral dosage forms as previously disclosed.

The present disclosure also provides a kit comprising a device for temporary residence in the stomach of a subject suffering from a condition which may benefit from local dispensing in the gastric region of an active pharmaceutical ingredient (API), the device having an expanded configuration, wherein in the expanded configuration, the device is configured to be retained fin the stomach of the subject when the device is positioned in the stomach, the device carries a load of the API and is configured for releasing at least partially the API while allowing chyme flow; and the device is further configured to be capable of transferring from the expanded configuration into an emptying configuration in which the device is configured to pass through the pyloric valve.

In some embodiments, the kit further comprises a conjunct emptying oral dosage form, the conjunct emptying oral dosage form comprising an effective amount of an emptying agent, wherein the device and the conjunct emptying dosage form are configured so that release of the emptying agent causes the device to transfer into the emptying configuration.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B illustrate perspective views of a device according to embodiments of the present disclosure respectively in an expanded configuration and in a closed configuration.

FIGS. 2A-2B show perspective views of the device shown in FIG. 1 in the expanded configuration.

FIGS. 3A-3B show a perspective view and a cross section view of a coupling head for use in a biasing assembly of the device shown in FIG. 1.

FIGS. 4A-4B show a front view and a cross section view of a trigger assembly cooperating with a biasing assembly for use in a device according to embodiments of the present disclosure.

FIGS. 5A-5B show a perspective view and a cross section view of the device of FIG. 1 in an emptying configuration.

FIGS. 6A-6B show cross section views of another example of a device according to embodiments of the present disclosure.

FIG. 7 shows a cross section view of another example of a device according to embodiments of the present disclosure.

FIGS. 8A-8F illustrate another example of a device according to embodiments of the present disclosure.

FIGS. 8G-8J illustrate a disassembling of the device according to embodiments of the present disclosure.

FIGS. 8′A-8′F illustrate another example of a device according to embodiments of the present disclosure.

FIGS. 9A-9F illustrate another example of a device according to embodiments of the present disclosure.

FIGS. 9′A-9′F illustrate another example of a device according to embodiments of the present disclosure.

FIGS. 10A-10F illustrate another example of a device according to embodiments of the present disclosure.

FIGS. 11A-11H illustrate another example of a device according to embodiments of the present disclosure.

FIG. 12 shows a device useful in a method of determining in vitro pyloric valve retention capability of a device in accordance with embodiments of the present disclosure.

FIG. 13 shows a device useful in a method of determining in vitro a capability of the device of being stopped by the pyloric valve while allowing chyme flow.

FIGS. 14A-14G illustrate an example of a device to allow chyme flow of which capability may be determined in vivo on a pig.

FIGS. 15A-15B illustrate a dosing system prepared for an in vivo study on a pig.

FIG. 16 is an Xray image of an exemplary device in the stomach of a pig.

FIG. 17 is a graph that illustrates the retention of an exemplary device in the stomach and rapid exit of dummy tablets through the GI of a pig.

### DETAILED DESCRIPTION OF EMBODIMENTS

Described herein are some examples of devices and methods useful for local drug release at the gastric region of a patient (human, or particularly adult human or young human).

In the following detailed description, numerous specific details are set forth in order to provide a thorough understanding of the subject matter. However, it will be understood by those skilled in the art that some examples of the subject matter may be practiced without these specific details. In other instances, well-known methods, procedures and components have not been described in detail so as not to obscure the description.

As used herein, the phrase “for example,” “such as”, “for instance” and variants thereof describe non-limiting examples of the subject matter.

Reference in the specification to “one example”, “some examples”, “another example”, “other examples, “one instance”, “some instances”, “another instance”, “other instances”, “one case”, “some cases”, “another case”, “other cases” or variants thereof means that a particular described feature, structure or characteristic is included in at least one example of the subject matter, but the appearance of the same term does not necessarily refer to the same example.

It should be appreciated that certain features, structures and/or characteristics disclosed herein, which are, for clarity, described in the context of separate examples, may also be provided in combination in a single example. Conversely, various features, structures and/or characteristics disclosed herein, which are, for brevity, described in the context of a single example, may also be provided separately or in any suitable sub-combination.

As used herein, the terms “about” and “at or about” mean that the amount or value in question can be the value designated some other value approximately or about the same. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

In the present application, the following terms and their derivatives may be understood in light of the below explanations:

As used herein, the terms “local dispensing” and “local release” and their derivatives may be used to refer to a regio-specific release/dispensing of a substance at a predefined location in the GI tract, in particular at the gastric region.

The term “swallowable” may be used to refer to an object having dimensions enabling oral administration to a human subject. The term oral administration may refer to ingesting the object. A swallowable object may be characterized as an object being capable of being fitted in a cylinder, wherein a length of the cylinder is, for example, about 35 mm or less and a diameter of the cylinder is, for example, about 12 mm or less.

As used herein, the term “gastric region” may be used to refer to a region substantially consisting of the stomach, and for example including the pyloric valve.

As used herein, the term “retained in the stomach” means that the device, in the expanded configuration and after reaching the pyloric region, does not pass through the pyloric valve and rather stays in the stomach. In other words, the device remains positioned within the stomach though the device may make movements inside the stomach.

As used herein “structural rigidity” may refer to the ability of an object to maintain its shape while being exposed to an external load.

The term “environmental conditions” may refer to biological, physical and/or chemical conditions of an environment i.e., a medium or milieu in which the device is positioned or intended to be positioned. The environmental conditions may include for example, temperature, pH, atmospheric pressure, gravity, electromagnetic field, vibration, glucose concentration, oxygen concentration, enzyme concentration, etc. In a predetermined part of the GI tract, for example the stomach or pyloric region, standard environmental conditions may refer to average physiological conditions observed in said part of the GI tract. For example, standard pH conditions for different parts of the human GI tract are summarized in the table below:

| | pH along the GI tract (1) | | |
|---|---|---|---|
| | | 25th | 75th |
| GI segment | median | percentile | percentile |
| stomach | 1.5 | 1.1 | 1.8 |
| duodenum | 6.4 | 5.7 | 6.9 |
| proximal small | 6.6 | 6.2 | 7.1 |
| intestine | | | |
| mid small intestine | 7.1 | 6.6 | 7.4 |
| distal small intestine | 7.3 | 7.0 | 7.5 |
| (including ICV) | | | |
| cecum | 5.7 | 5.2 | 6.3 |
| ascending colon | 5.6 | 5.1 | 6.5 |
| transverse colon | 5.8 | 5.3 | 6.4 |
| descending colon | 6.6 | 6.3 | 7.0 |
| rectum | 6.6 | 5.8 | 7.1 |

As used herein “degradability” may refer to the ability of a device to lose structural rigidity under certain physiologic conditions. The degradation products may be excretable and/or absorbable by the body.

As used herein, the term “closed configuration” of a device may be the state of the device prior to administration where the device has a size that is suitable for swallowing or for oral manipulation with an endoscope or suitable for administration by colonoscopy (lower endoscopy). The closed configuration may also be referred to as a swallowing or swallowable configuration, collapsed configuration, compact configuration, compressed configuration, deflated configuration, folded configuration or the like.

As used herein, the “expanded configuration” of the device may be the state of the device when it resides in the stomach. In some embodiments, the device may be orally administered, for example via ingestion or via upper endoscopy, and the device may be delivered in the expanded configuration in the stomach. The device may be transited by the GI motility to the pyloric region and in the vicinity of the pyloric valve. The expanded configuration of the device notably prevents passage of the device through the pyloric valve. The expanded configuration of the device may not block the pyloric valve and may enable chyme flow therethrough. The expanded configuration may also be referred to as opened configuration, inflated configuration, unfolded configuration or the like.

The devices and methods discussed herein thus provide gastric retentive drug devices and dosage forms that are useful for local drug release at the gastric region, based on systems with expanding geometry through unfolding. The system or dosage form are initially in a condition or configuration suitable for swallowing. Then, the system or dosage form may expand in the stomach to prevent being released from the stomach. Eventually, the system or dosage form may reduce in size to pass through the pylorus valve or disassemble or disintegrate. The devices and methods discussed herein may provide the following aspects: rapidly expand in a sufficiently short time; stay in the stomach for a predetermined residence time period, such that releasing of the drug can be efficiently achieved; and maintain a sufficiently large size throughout the residence time period under gastric or physiology conditions under fasted or fed states. The devices and methods discussed herein may comprise an emergency release mechanism to expel the delivery system or dosage form in an emergency situation.

The expression “carrying a load of an active pharmaceutical ingredient” as used in the present disclosure refers to the fact that the device securely contains a quantity of said API in the state of the device prior to administration (e.g., the closed configuration). By “securely”, it is meant that prior to administration (e.g., the closed configuration), the device securely contains a quantity of said API located inside a convex hull of the device, and said API is not extractible unless deforming the outer shape of device, due to physical barriers formed all around the API by components and/or material. In other words, the device may carry an embedded dosage form including an API, i.e., a therapeutic dosage form. Thus, the device is enabled to be administered and securely carry/transport with itself a certain amount of the API to the stomach. Then, when the device is in the expanded configuration, the device is configured for releasing at least part (e.g., all of) said (initially carried) load of the API. Indeed, the device is configured for the API to be exposed to chyme flow so as to release the API, at least when the device is in the expanded configuration and positioned in the stomach. The release may optionally be over a certain period of time, e.g., longer than one minute, ten minutes, thirty minutes, one hour, two hours, five hours, twelve hours, one day, or one week. In other words, the API load may be configured to erode, diffuse, or dissolve from within the device.

The device may present a structural rigidity substantially independent of the load of the API. The device may notably maintain its expanded shape while the API is being released, and in particular even if the API has been fully released. In particular, the device may be configured such that, when administered to a subject but without any load of API, the device is structurally rigid enough to be retained in the stomach of the subject. As such, the load of the API may be unsupportive of the device's structure.

The device may carry a load of the API in any manner, and optionally in several different manners. The device may embed at least part of the load of the API inside (e.g., enclosed in) or on (e.g., mounted onto or attached to) one of its components. Said at least part of the load may remain embedded in the device. In other words, said at least part of the load may be securely attached to the device even in the expanded configuration, such that said at least part of the load is not extractible (apart from release of the API) unless exerting a force above a predetermined threshold. Alternatively or additionally, at least part of the carried load may be detachable from the device when the device deforms into the expanded configuration.

The device may for example comprise an inner (available) space in the closed configuration. The inner space may contain (e.g., lodged therein and/or unattached) at least part of the load of the API. The inner space may be an interstice left between components of the device. The inner space may be an interstice left (i.e., present) between or flanked by components of the device. Such examples optimize space left between components of the device by using it for API loading. In examples, the device may comprise at least two flexible arms (e.g., at least three or at least four), each arm having a first end and a second end, a first and second coupling heads, wherein the first end of each arm is coupled to the first coupling head and the second end of each arm is coupled to the second coupling head, and a resiliently deformable member configured to force the coupling heads together to bend the arms thereby biasing the device in the expanded configuration. In such examples, the inner space may be formed in the closed configuration around the resiliently deformable member and between the arms and the coupling heads. In options, the device may further comprise a support tube, the resiliently deformable member being arranged inside the support tube. In such options, the inner space may comprise an interstice formed between the support tube and the resiliently deformable member. In examples of such options, the support tube may have apertures (on its peripheral wall) which provide exposure (i.e., fluid communication with the physiological environment surrounding the device) to the cavity. This allows fine control of the API release. In particular, the apertures may present a design which provides a predetermined release rate. Alternatively or additionally, the exposure apertures may be coated, so as to expose the contained API load only when desired, or on the contrary uncoated. Alternatively or additionally, the exposure apertures may be coated so as to expose the contained API load only when desired, for example when the device reaches the stomach. Alternatively, the exposure apertures may be uncoated.

Alternatively or additionally, at least one material portion or at least one component of the device may comprise an exposed cavity (i.e., a substantially void space inside a portion of material and having a peripheral wall substantially enclosing the space). In such a case, the exposed cavity may contain (e.g., enclosed therein and/or unattached) at least part of the load of the API. The cavity is open to the device environment, at least when the device is in the expanded configuration. Thus, the load of the API may flow/erode from the cavity when the device is in the expanded configuration. The cavity may be formed within a peripheral wall having apertures which provide exposure of the exposed cavity. Such examples allow simple manufacturing and fine control of the API release. In particular, the apertures may present a design which provides a predetermined release rate. Alternatively or additionally, the exposure apertures may be coated, so as to expose the contained API load only when desired, or on the contrary uncoated. In examples, the device may comprise at least two flexible arms (e.g., at least three or at least four), and one or more (e.g., all) of the arms may comprise such cavity (e.g., within one or more—e.g., each —arm section when the arm is articulated and/or composed of a set arm sections).

Alternatively or additionally, at least one material portion or at least one component of the device may comprise an exposed recess. In such a case, the exposed recess may lodge (i.e., be filled with) at least part of the load of the API (e.g., press-fitted in the recess, or formed by molding inside the recess). Such examples allow simple manufacturing. In examples where the device comprises a support tube, the support tube may have one or more peripheral groove recesses. Each peripheral groove may lodge the API, for example a ring-shaped (e.g., solid) form containing the API. Alternatively or additionally, the arms may comprise exposed recesses. Each exposed recess may be coated at its opening, so as to expose the contained API load only when desired, or on the contrary uncoated.

Alternatively or additionally, at least one material portion or at least one component of the device may comprise a coating on an exposed surface, the coating containing at least part of the load of the API. For example, each arm section and/or a support tube may be coated with any mixture containing the API.

The load of the API may present any texture, shape, and/or composition. For example, the API may be contained in a solid form, in a semi-solid form, as powder, as a gel texture, and/or in a liquid. In addition, the API may be contained in one texture, shape, and/or composition at one location, and in another texture, shape, and/or composition at another location. Furthermore, the device may carry several APIs, for example cooperating together to treat a medical condition. The load of the API may contain one or more pharmaceutically acceptable excipients. The one or more excipients may be inactive and/or include one or more of filler, binder, lubricant, diluent, preservative, control release agent, disintegrant (e.g., sodium, starch, glycolate) and the like. Solid forms of the API may include one or more tablets, and/or pellets. The carried tablets (e.g., pills) may have common tablet shapes such as round, standard convex, compound cup, oval, bullet, triangle, diamond, etc., so as to fit the cavity, recess, groove, inner space designed to accommodate the tablet. The solid forms may be coated or uncoated.

The device may be unable to release the API (at all) when the device is in the closed configuration. The device may further be optionally unable to release the API unless the device is in the expanded configuration. For example, in some embodiments of the present disclosure the device may comprise a container in which the device can be fitted in the closed configuration, and the container may form a physical barrier preventing release of the API carried by the device prior to the container dissolving and the device thereby transferring from the closed configuration into the expanded configuration. In some embodiments, the device includes an API useful in treating a disease or disorder for which dispensing of the API in the gastric region is beneficial.

The device may carry any quantity of the API. For example, the load of the API (e.g., possibly including excipients) may occupy at least 5%, 10%, 15%, 20%, or 25% of the volume of a convex hull of the device in the closed configuration. In other words, the API may be contained in an embedded dosage form or formulation, and the form/formulation, and the device may offer in the closed configuration enough free space such that at least 5%, 10%, 15%, 20%, or 25% of the volume of its convex hull is occupied by (accommodated with) the form/formulation. The type and quantity of API load may be determined by a healthcare professional.

The term “dosage form” as used in the present disclosure refers to solid dosage forms which may include an emptying agent for modifying the gastric environmental conditions to cause the device according to some embodiments of the present disclosure to transfer from the expanded configuration into the emptying configuration. Such a dosage form may optionally include one or more pharmaceutically acceptable excipient. The dosage forms may be referred to as “emptying dosage forms”. In some embodiments, the emptying agent is a pharmaceutically acceptable ingredient in an amount effective to cause transfer of the device into the emptying configuration. In some embodiments the emptying agent is a basic ingredient including aluminum hydroxide, magnesium carbonate, calcium carbonate sodium bicarbonate and the like. The dosage forms (emptying agent) may include, for example, tablets, pellets or capsules. Tablets may have common tablet shapes such as round, standard convex, compound cup, oval, bullet, triangle, diamond, etc. Capsules may carry a solid (e.g., tablet, particles, granulates) or liquid load. Dosage forms generally have dimensions such as to fit into a cylinder having a length from 10 mm to 30 mm and a diameter from 7 mm to 12 mm. In some embodiments, the dosage form comprises a mixture of active ingredient(s) (emptying agent) and inactive excipients including one or more of filler, binder, lubricant, diluent, preservative and the like.

In some embodiments the dosage form may present any form that can be dosed to the subject, such as a gel, a solid, a semi-solid, or a liquid form, or any combination thereof.

In some embodiments, the emptying dosage form may be an emptying tablet, that is, an oral dosage form for cooperation with the device to trigger emptying, when the trigger assembly is configured to activate when the device in the expanded configuration is exposed to a surrounding environment of a pH being above a predetermined threshold of, for example pH 6). The emptying dosage form may be designed to transit to the stomach, be retained in the stomach, release its basic payload (emptying agent) thereby increasing the surrounding environment pH, enabling activation of a trigger assembly of the device (e.g., erosion of—e.g., enteric polymers such as Eudragit® L or S-pins as support elements; methacrylic acid-methyl methacrylate (MMA) copolymers) and causing transfer of the device into the emptying configuration. An exemplary emptying tablet composition includes a basifying agent.

The term “external envelope” may be used to refer to a hull separating internal and external portions of the device. For example, the external envelope of the device may comprise an outer surface of the device.

The term “pharmaceutically acceptable” refers to a material that is not biologically or otherwise unacceptable when used for the purposes of the present disclosure. For example, the term “pharmaceutically acceptable carrier” refers to a material that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration. The materials used for manufacturing the device in accordance with the present disclosure may be pharmaceutically acceptable.

The term “pH dependent polymer” may refer generally to a polymer whose degradability or dissolubility changes depending on the pH of a surrounding solution. For example, in some embodiments of the present disclosure a trigger assembly may include one or more support elements which may be at least partially made of a pH dependent polymer so that the one or more support elements do not degrade at a standard stomach environmental conditions (i.e., stomach standard pH of about 1-3) while do degrade at a less acidic pH (e.g., pH of about 6.0 or 7.0 or 8.0). A type of pH dependent polymer is an enteric polymer. An enteric polymer may be understood as a polymer that does not readily dissolve or degrade under the typical pH and other physical conditions of a human stomach, but that does dissolve or degrade at pH and other physical conditions of the intestinal tract of a human, i.e., the conditions that exist following passage from the stomach through the pylorus (i.e., pH>5). For example, in some embodiments of the present disclosure the device may comprise a container in which the device can be fitted in the closed configuration and the container may dissolve in the stomach. In some embodiments, as described below, the trigger assembly may include one or more support elements at least partially made of an enteric polymer or of a pH dependent polymer. When the singular form of “pH dependent polymer” is used, this can refer to one enteric polymer, a mixture of two or more enteric polymers, or a mixture of polymers of which at least one is a pH dependent polymer, as long as the resulting mixture is pH dependent in nature.

The load of API carried by the device may be distinct and separate from the support elements (e.g., timers). Each support element may be integrally formed, and the load of the API may be elsewhere. In examples, the support elements may be located inside the coupling heads, while the carried load of the API may be located between the coupling heads and the arms (i.e., outside the coupling heads).

Local release of therapeutic agents in the gastric region may have therapeutic benefit when used in various medical conditions. Non limiting examples include:
a. Treatment of a gastric ulcer, or gastroesophageal reflux disease. Effective treatment may be achieved when local and durable the drug in the stomach is obtained (i.e., high local concentration and long exposure duration).
b. Treatment of other stomach disease, including stomach cancer and gastric microbial diseases. Examples of stomach cancer drugs include including angiogenesis inhibitors, check point inhibitors, metabolite inhibitors etc. Stomach infections may be caused, for example, by parasites, bacteria (such as H. pylori, viruses, fungi. Useful anti-microbials include amoxycillin neomycin, rifaximin, anthracyclines, bleomycins, mitomycin, dactinomycin, and plicamycin. A health care professional may identify a relevant antibiotic for a particular disease, from for example, the WHO publication “Critically important antimicrobials for human medicine”, (ISBN: 978-92-4-151552-8; 6th revision).
c. Improved systemic therapeutic exposure of the APIs (e.g., absorption and bioavailability) which may enable achieving systemic plasma concentrations having minimal fluctuations enabling better efficacy and safety. For example, to improve absorption in drugs having short absorption window such as Dopa agonists for Parkinson's or to lower risk of adverse effect in drugs having low therapeutic index such as anti-cancer (e.g. ibrutinib) and steroids.
d. Improvement in patient adherence in which the device may be designed to be retained for prolonged time in the stomach while releasing API. Such treatments lower the amount of drug dosing per day and improve the adherence to treatment for both the patient and the care givers (for example, anti-psychotic drugs, anti-HIV medications, etc.).

An API may include any substance relevant for gastric retention as recognized in the art which may be therapeutic, diagnostic or otherwise beneficial. A list of relevant APIs may comprise APIs which act locally in the stomach, APIs with primarily absorption in the stomach, and APIs which are poorly soluble in alkaline pH. The list may further comprise APIs with narrow windows of absorption, APIs with rapid absorption from the GI tract, APIs that degrade in the colon, and APIs that disturb colonic microbes.

Examples of the API contained in the load carried by the device are now discussed. In some embodiments, the API is a small molecule. In some embodiments, the API is a prodrug. In some embodiments, the API is a pharmaceutically acceptable salt of an API. In some embodiments, the API comprises amino acid or nucleotides. In some embodiments, the therapeutic agent may be a combination of two or more therapeutic agents. A non-limiting list of APIs includes anti-retroviral agents; CYP3A inhibitors; CYP3A inducers; protease inhibitors; adrenergic agonists; anti-cholinergics; mast cell stabilizers; xanthines; leukotriene antagonists; glucocorticoids treatments; local or general anesthetics; non-steroidal anti-inflammatory agents (NSAIDs, e.g., naproxen); antibacterial agents; anti-fungal agents; sepsis treatments; steroidals; local or general anesthetics; monoamine oxidase inhibitors; hypothalamic phospholipids; endothelin converting enzyme (ECE) inhibitors; opioids; thromboxane receptor antagonists; potassium channel openers; thrombin inhibitors; growth factor inhibitors (e.g., modifiers of EGF, PDGF activity); anti-cytokines (e.g., anti-TNF activity); anti-platelet agents (e.g., aspirin); anticoagulants; heparins; renin inhibitors; neutral endopeptidase (NEP) inhibitors; vasopepsidase inhibitors (dual NEP-ACE inhibitors); HMG CoA reductase inhibitors (e.g., statins); squalene synthetase inhibitors; fibrates; bile acid sequestrants; anti-atherosclerotic agents; MTP Inhibitors; calcium channel blockers; potassium channel activators; alpha-muscarinic agents; beta-muscarinic agents; antiarrhythmic agents; diuretics; thrombolytic agents; anti-diabetic agents; mineralocorticoid receptor antagonists; aP2 inhibitors; phosphodiesterase inhibitors; protein tyrosine kinase inhibitors; antiproliferatives; immunosuppressants; antimetabolites; antibiotics; enzymes; farnesyl-protein transferase inhibitors; hormonal agents (e.g., cortisone); microtubule-disrupter agents; plant-derived products (e.g., taxanes); topoisomerase inhibitors; prenyl-protein transferase inhibitors; cyclosporins. Listings of additional examples of known therapeutic agents can be found, for example, in the United States Pharmacopeia (USP), Physician's Desk Reference (Thomson Publishing), and/or The Merck Manual of Diagnosis and Therapy, 20th ed. (2018) Robert S. Porter, ed., Merck Publishing Group, or, in the case of animals, The Merck Veterinary Manual, 11th ed., Susan E. Aiello and Michael A. Moses eds., Merck Publishing Group, 2016; and “Approved Drug Products with Therapeutic Equivalence and Evaluations,” published by the United States Food and Drug Administration (F.D.A.) (the “Orange Book”).

Further provided herein is a method of treating a condition which may benefit from local dispensing of an API in the stomach of a subject suffering from the condition. The treatment includes orally administering to the subject a device as described herein, thereby treating said condition in said subject.

“Treating” as used herein encompasses, e.g., inducing inhibition, regression, clinical remission or stasis or inhibiting, reducing the severity of, eliminating or substantially eliminating, or ameliorating a symptom of the disease or disorder, e.g., a gastric ulcer, and gastroesophageal reflux disease, lowering Parkinson's disease related symptoms.

“Inhibition” of disease progression or disease complication in a subject means preventing or reducing the disease progression and/or disease complication in the subject.

A “symptom” associated with a disease or disorder disclosed herein includes any clinical or laboratory manifestation associated with any relevant disease or disorder and is not limited to what the subject can feel or observe. A non-limiting example includes the following for gastric ulcers. weight loss diarrhea, nauseas, vomiting, abdominal cramps and aches, abdominal pain, inflammation, tiredness, anemia, perforations. Symptoms of other conditions are known in the art and are identified by a medical practitioner.

The device according to the present disclosure may have any physical configuration that is compatible with certain basic functionalities.

First, it is configured for being administered to a human. For example, the device can be deformed (e.g., folded) from an expanded configuration into a closed configuration and locked by a locking element (e.g., a capsule) so as to be sized and shaped for oral administration i.e., ingestion. The locking element may substantially maintain the device integrity prior to entry into the stomach. The locking element may dissolve after (e.g., within 1 or 10 minutes) exposure to stomach environmental conditions. The device may also, in some embodiments, be configured to be positioned by endoscopy i.e., by gastroscope —in the stomach. In some embodiments, the device in the collapsed configuration may be manipulated with the endoscope and expanded in the stomach. In some endoscopically administered embodiments, the device may be inflatable between a collapsed/deflated configuration and the expanded configuration. The device may be positioned in the stomach in the collapsed configuration and inflated in the expanded configuration thereafter. In some upper endoscopy administered embodiments as well as in some orally administered (ingested) embodiments, the device may be administered to the subject under fasted conditions.

Second, in orally administered/ingestible embodiments, the device is configured for being capable of transiting through the GI until the stomach. For example, the device may be contained in a capsule which does not dissolve before being in standard stomach environmental conditions (fasted and/or fed). Further, the device in the expanded configuration may be configured to enable chyme flow into the small intestine while moving in the stomach.

Third, the device in the expanded configuration is configured for retention in the stomach (i.e., not to pass through the pyloric valve) in standard GI motility conditions. For example, the device in the expanded configuration may be sized and/or shaped and/or have a structural rigidity that prevents passage through the pyloric valve of a standard subject under standard GI motility conditions.

Fourth, the device is configured for being capable of transferring into an emptying configuration which allows passage through the pyloric valve, and optional excretion through the subject's body. For example, the device may include a trigger assembly causing the device to transfer into the emptying configuration when activated. The trigger assembly may be configured to be activated when the device in the expanded configuration is exposed to an activation signal such as being exposed to standard stomach environmental conditions for a predetermined residence time period or such as a surrounding environment reaching a predetermined set of environmental conditions (e.g., a pH threshold). The device may be configured to include one or more structural weak points which may cause the device to swiftly fall apart into the emptying configuration when the trigger assembly is activated.

In some embodiments, the device may be used in conjunction with another means able to trigger the device to transfer into an emptying configuration. For example, the means may comprise an emptying dosage form. Alternatively, the means may comprise an oral lead such as a catheter or endoscope that can be placed close to the stomach (e.g., pylorus region). The oral lead may deploy an amount of an emptying agent sufficient to cause the stomach environment to reach at least one activation environmental condition thereby causing activation of the trigger assembly and transfer of the device into the emptying configuration. In examples of this embodiment, the means can be used in case of emergency, e.g., when the pyloric valve is obstructed.

Fifth, the device in the expanded configuration is configured to release at least partially the carried API, thus enabling a retained treatment within the stomach of a desired duration. The device may allow chyme flow through the pyloric valve while releasing at least partially the carried API.

In particular, in embodiments where the device comprises at least two (e.g., at least three or at least four) flexible arms, two coupling heads, and a resiliently deformable member, the flexible arms, coupling heads, and resiliently deformable member may be dimensioned and/or arranged to allow chyme flow in an inside space, e.g., formed around the resiliently deformable member between the arms and the coupling heads, while the API is being released. The chyme flow in the inside space may improve the release of the API by providing a more uniform diffusion for the API. In particular, in such embodiments, the device may carry/embed the API such that it is exposed at least to said inside space.

Furthermore, the device according to some embodiments of the disclosure may have in the expanded configuration a convex hull with a high sphericity (e.g., larger than 0.8) and/or a low ratio between a maximal circumference and a minimal circumference (e.g., below 1.5). This high sphericity of the expanded structure improves tissue pressure distribution. The device according to the present disclosure may have a particularly high (convex hull) volume ratio of the expanded configuration (i.e., the form in which the device is retained in the stomach) relative to the closed configuration (i.e., the form in which the device is administrated). This high ratio enables the retainability of the device in the stomach while enabling easier swallowability.

The device according to the present disclosure may be simple to manufacture, and its design (e.g., the arms, the coupling heads, and the resiliently deformable member) may provide some flexibility while it is in the expanded configuration, so as to avoid damaging the body tissue. In addition, the device may be relatively fast to expand thanks to such a configuration which increases the probability of retention following dosing under fasted gastric condition.

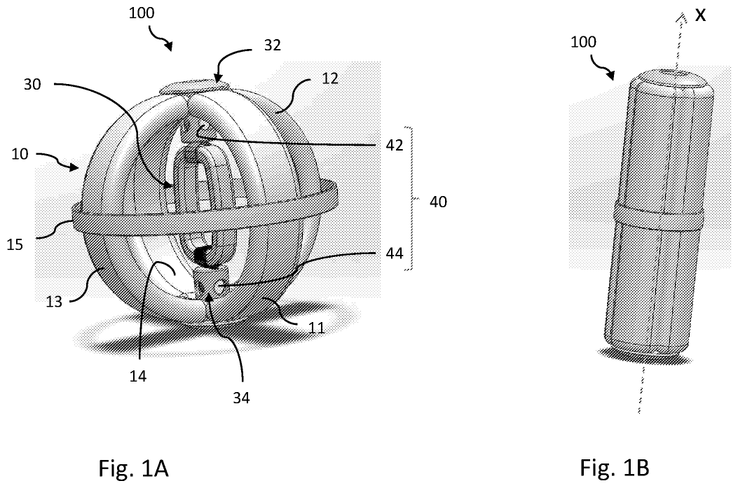

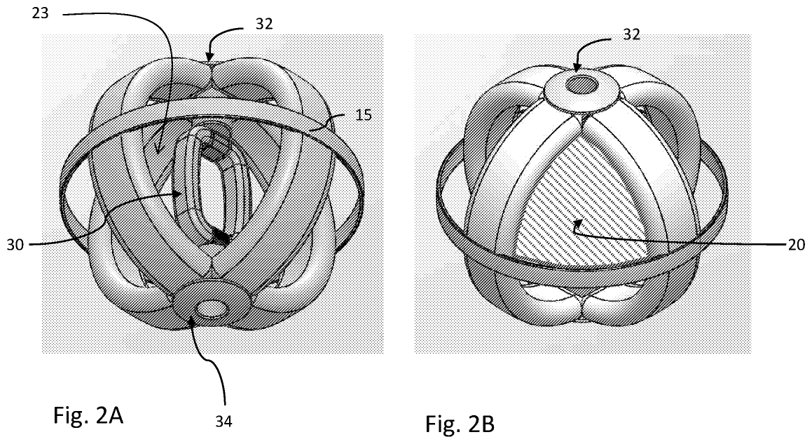

FIGS. 1A and 1B show an example of a device 100 according to the present disclosure respectively in an expanded (open) configuration and in a swallowing (closed) configuration. FIGS. 2A-5B further shows the device 100.

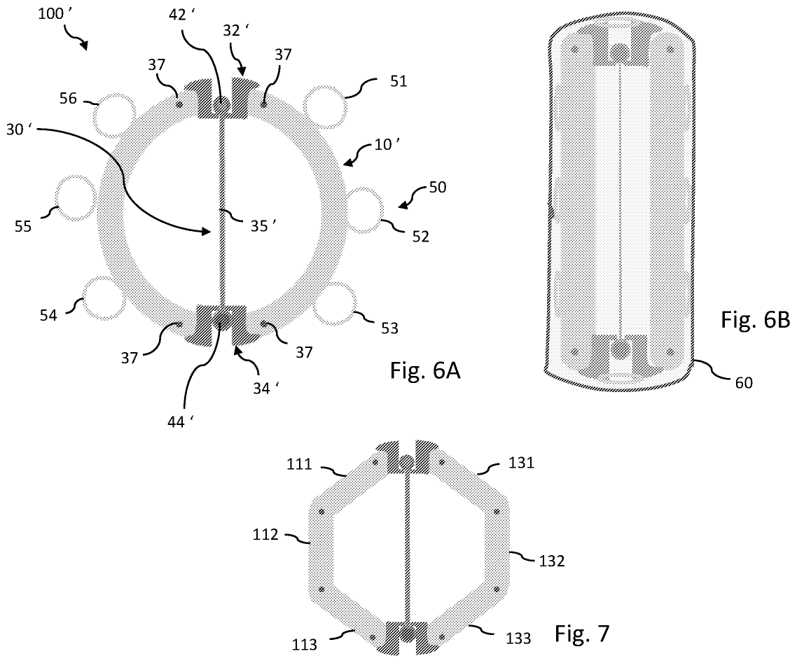

The device 100 is intended to be orally administered—in the closed configuration —to a patient for temporary residence—in the expanded configuration—at the stomach of said patient. In some embodiments, the device in the closed configuration may be capable of passing through the pyloric valve of the patient. In some embodiments, the external envelope of the device in the closed configuration has dimensions enabling passage through the pyloric valve and the device in the emptying configuration has an external envelope similar to the device in the closed configuration (see FIG. 5A). Basically, the device in the closed configuration may be capable of being fitted into a cylinder of about 35 mm length or smaller and of about 12 mm diameter or smaller.

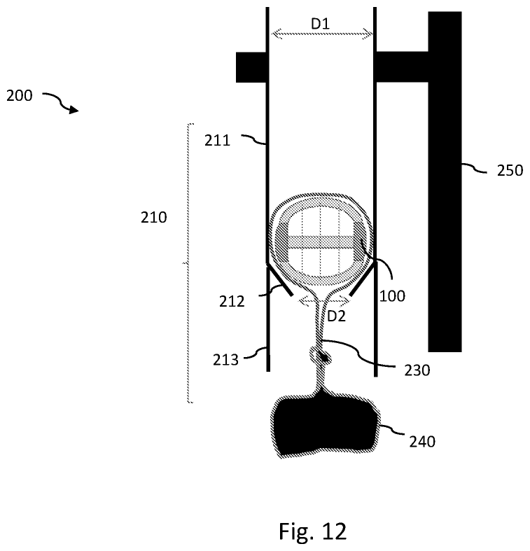

In the expanded configuration, the device 100 is configured to be retained in the stomach and resist standard GI motility. In other words, the device 100 may be configured to not pass through the pyloric valve of the patient when it is positioned in the stomach. The device 100 may include a flexible frame. The device 100 in the expanded configuration may have an uncompact shape while the device in the closed configuration may have a compact shape. The device 100 in the expanded configuration may have a spherical or ovoid, tetrahedral, cuboidal or semi spherical outer shape. In some embodiments, the dimensions of the device in the expanded configuration may be such that the device cannot pass through an orifice of about 17 mm diameter and preferably of about 20 mm. Generally, the device 100 may have a shape and/or a size and/or a structural rigidity enabling the device to be retained in the stomach. The capability of the device 100 to be retained in the stomach may be defined in accordance with methods described in details herein below with reference to FIG. 12.

The device 100 is further configured to transfer into an emptying configuration in which it can pass through the pyloric valve under standard GI motility conditions. In some embodiments, the capability of the device 100 to pass through the pyloric valve in the emptying configuration may be defined in accordance with methods described in detail herein below with reference to FIG. 12.

The device 100 may be deformable from the closed configuration of FIG. 1B into the expanded configuration of FIG. 1A.

The device 100 may comprise a body 10, an opening assembly in the form of a biasing assembly 30 and a trigger assembly 40.

The body 10 may be formed of several (e.g., four) flexible arms 11-14 joined to each other by both ends. The body may form the frame of the device 100. The biasing assembly 30 may be configured to bend the flexible arms 11-14 in a rounded shape. When not bent (i.e., when the biasing of the biasing assembly is overcome), the arms 11-14 may substantially extend in the direction of a longitudinal axis X. When not bent (i.e., when the biasing assembly 30 is disabled), the arms 11-14 may be disposed to form a hollow tube. The arms 11-14 may be made of silicone. In some embodiments, the body 10 may be made from a hollow tube of silicone comprising four longitudinal slits.

The device 100 may further comprise an optional circumferential belt 15 disposed around the four flexible arms. The circumferential belt 15 may be disposed in a plane substantially perpendicular to the longitudinal axis X. The circumferential belt 15 may be made of an elastic material so as to allow the flexible arms 11-14 to open into the expanded configuration without substantial restriction. For example, the arms and the circumferential belt may be made of silicone of different thicknesses or different grade (e.g., different shore A). For example, the flexible arms may be made of silicone shore 80A and the peripheral belt may be made of silicone shore 50A. Generally, the device may be configured for remaining in the closed configuration for a shelf lifetime period of about 2 to 52 weeks and maintain its functionalities. The device may be made of a material with low creep (i.e., low tendency to have gradual permanent deformation under the influence of persistent mechanical stresses, e.g., the stomach wall pressure). The circumferential belt 15 may alternatively be made of a material that is flexible but not elastic.

The circumferential belt 15 may have a structural function, for example stabilize the platform formed by the device 100 when open in the expanded configuration. This improves stomach retention.

When the device 100 is in the open (expanded) configuration, the body 10 and the circumferential belt 15 may form a tridimensional meshed structure. The openings 20 (see FIG. 2B in which one of opening 20 is illustrated by a dashed surface) formed between the flexible arms 11-14 and the circumferential belt 15 in the open configuration may define a mesh of said meshed structure. The openings 20 may be configured to enable chyme flow.

The biasing assembly 30 may be configured to resiliently hold (bias) the device 100 in the expanded configuration. In other words, the biasing assembly 30 may be configured to cause the device to tend to resiliently return from the closed configuration into the expanded configuration.

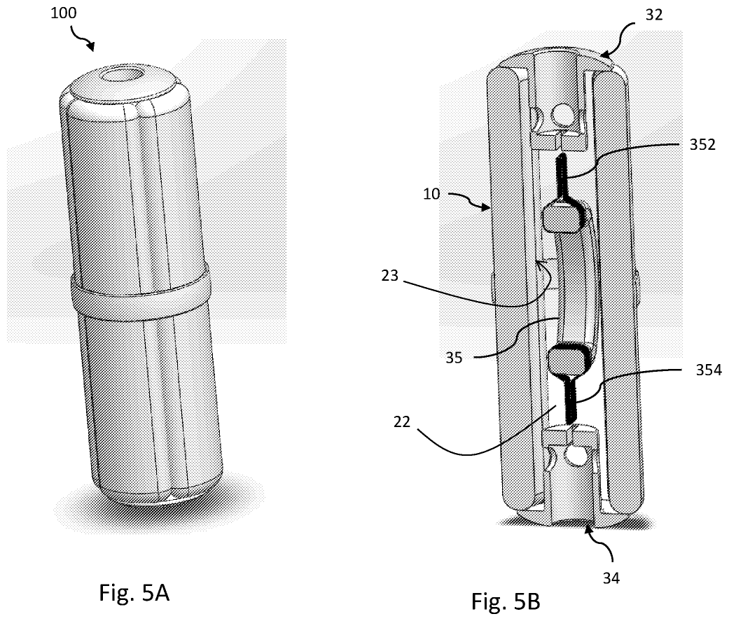

The biasing assembly 30 may comprise two coupling heads 32, 34 and an elastic member 35. A first end of each flexible arm may be coupled to the first coupling heads 32 and a second end of each flexible arm may be coupled to the second coupling head 34. More details regarding the arrangement of the coupling heads 32, 34 and the elastic member 35 are given hereinafter with reference to FIGS. 4A-4B.

The trigger assembly 40 is configured to cause the device 100 to transfer from the expanded configuration into the emptying configuration upon activation. The trigger assembly 40 comprises two support elements 42, 44 configured to temporary maintain the device into the expanded configuration. The support elements 42, 44 are configured to be disabled when the device in the expanded configuration is exposed to a predetermined activation signal. The support elements 42, 44 are configured to cooperate with the biasing assembly 30 so that, when the support elements 42, 44 are disabled, the biasing assembly 30 is irreversibly disabled and the device 100 transfers-into the emptying configuration. The trigger assembly 40 may be configured to cause additional degrading of the device such as for example, disassembling into two or more subcomponents, additional decline of shape, size and/or structural rigidity.

For example, the support elements 42, 44 may be configured to be disabled after the device 100 is exposed to standard stomach environmental conditions for a predetermined residence time period for example of one day, two days, three days or more and/or twelve weeks or less (e.g., any time period from 1 to 12 weeks, such as one month, one week, two weeks, three weeks, or even one day or two days). In other words, the trigger assembly 30 may activate after the device is positioned in the stomach for a predetermined time period or after a predetermined time period elapsed subsequent to swallowing of the device. In some embodiments, support elements configured to be disabled after the device is exposed to standard stomach region environmental conditions for a predetermined residence time period may be made of a combination of a material degrading at standard stomach environmental conditions (such as Eudragit® EPO, an amino methacrylate copolymer that dissolves at pH<5) and a control release delay material (such as a polymer carrier).

The support elements 42, 44 may also or alternatively be configured to be disabled after the device is exposed to a predetermined set of activation environmental conditions. For example, the support elements 42, 44 may be configured to be disabled when a surrounding environment reaches (e.g., above) a predetermined pH threshold (e.g., pH 6, 7 or 8). For this purpose, the support elements 42, 44 may be erodible elements at least partially made of a material configured to dissolve (or degrade) when the surrounding pH reaches the predetermined pH threshold. In some embodiments the support elements 42, 44 may be at least partially made of a material dissolving at (i.e., when the pH reaches) the predetermined pH threshold. For example, the support elements may be made of a base-soluble polymer. For example, the support elements 42, 44 may be at least partially made of a material soluble at pH above 6 such as enteric polymer such as Eudragit® L or for example HPMCAS LG (hydroxypropylmethylcellulose (hypromellose) acetate succinate, L grade), or of a material soluble at pH above 7 such as Eudragit® S or for example HPMCAS HG (hydroxypropylmethylcellulose (hypromellose) acetate succinate, H grade).

In some embodiments, the support elements 42, 44 may have a pin shape. Pin-shaped support elements 42, 44 of Eudragit® L or S may be manufactured as follows: a Hot Melt Extrusion machine is set to for example about 120-140° C. HPMCAS LG powder is fed into the HME machine for example by a gravimetric feeder at a rate of 1 kg/hr. The HME machine snail speed is set to 100 rpm. The melted material is drawn from the HME machine, it is forwarded as strands onto a conveyor belt to cool. Once cooled, the strand is chopped by a chopping machine to a pin shape of about 1.5 mm diameter and 2 mm length.

In some embodiments, the support elements 42, 44 may be at least partially made of a material dissolving in standard stomach region environmental conditions, such as Eudragit® EPO, and coated with a material dissolving at the predetermined pH threshold, such as Eudragit® L or S. Such pin-shaped coated support elements may be manufactured as follows: The hot melt extrusion machine (HME) is preheated to for example about 120-150° C. The HME machine snail speed is set to 100 rpm. Powder of Eudragit® EPO is added into HME feeder and pushed by the snail outwards. As the melted material is drawn from the HME machine, it is forwarded as strands onto a cooling machine to cool on a conveyor belt. Once cooled, the strand is chopped by a chopping machine to pin shape of about 1.5 mm diameter and 2 mm length. The pins are then placed in a coating system (e.g., vector coater) and coated with Eudragit® L or S coating with a total of for example about 10% of weight gain.

In some embodiments, at least one of the support elements 42, 44 is configured to be disabled after the device is exposed to standard stomach environmental conditions for a predetermined time period, and at least one of the support elements 42, 44 is configured to be disabled after the device is exposed to a predetermined set of activation environmental conditions, for example a surrounding pH reaching above pH 6.

The support elements 42, 44 may be arranged to form keystones of the device in the expanded configuration so as to provoke a collapse of the device into the emptying configuration after the device is exposed to the predetermined activation signal. In other words, the support elements 42, 44 are arranged to form one or more structural weak points. When the support elements 42, 44 are disabled, the one or more structural weak points cause the device to fall apart in the emptying configuration. The support elements 42, 44 are arranged so that a transfer duration of the device from the expanded configuration into the emptying configuration is substantially smaller (e.g., equal or less than ½, ⅓, ¼, ⅕, ⅙, 1/7, ⅛, 1/9, 1/10, 1/100, 1/1000) than a standard residence time period (e.g., equal or more than 1, 2, 3, 4, 5, 6, 7 or 8 weeks). This provides an improved control of the device in in-vivo conditions. In particular, in embodiments in which the trigger assembly is activated by external means, this enables to quickly empty the device on-demand without requiring an endoscopy (gastroscopy) procedure even in urgent cases.

In the expanded configuration, the device 100 may have substantially an ovoid or spherical shape. A diameter of the device in the expanded configuration may be of about 15 mm to 35 mm, preferably of about 20 to 30 mm, or of about 25 to 30 mm. In the closed configuration, the device 100 may have substantially an elongated shape. A length of the device 100 may be of about 10 mm to 45 mm, preferably of about 30 mm. A diameter of the device 100 in the closed configuration may be of about 5-15 mm, preferably of about 9-12 mm.

The device 100 may further comprise a locking element (not shown) arranged to temporary maintain the device in the closed configuration to facilitate oral administration. The locking element may be configured to temporarily overcome the biasing assembly 30. The locking element may be configured to release the device in the stomach into the expanded configuration. In some embodiments, the locking element may be at least partially made of a material dissolving in stomach environmental conditions while not dissolving or having a slower dissolving rate in the esophagus so to enable safe swallowing. In some embodiments, the locking element may be a container (e.g., a capsule). The container may be made of or coated for example with Eudragit® EPO that dissolves in pH<5.

In some embodiments, the device may include a labelling element enabling detection by external detection means. For example, the device may include a barium (or a metal) labelling element which can be detected by detection methods like X-ray imaging.

The device 100 may be configured to transit within the stomach until the pyloric valve in the expanded configuration without damaging the patient tissue. The device 100 may have an external envelope configured to contact the patient tissue. In particular, the device 100 may be flexible to avoid damaging the patient tissue. The external envelope of the device 100 may be blunt (unsharpened). A maximum diameter of the device in the expanded configuration may be below about 35 mm and preferably below about 30 mm or below 25.

The device may operate according to the general operation described below. As explained, the device in the closed configuration may be temporarily maintained in the closed configuration by a capsule as locking element. Following oral administration, the capsule may dissolve in the stomach thereby releasing the device into the expanded configuration. Alternatively, the device may be positioned in the expanded configuration in the stomach by upper endoscopy (gastroscope). The device in the expanded configuration moves in the stomach and may at times transit to the pyloric valve region due to GI motility.

The device 100 carries a load of an API (not shown on the figures), and the device 100 is configured for releasing at least partially the API while allowing chyme flow, when the device is positioned in the stomach and in the expanded configuration.

The load may be solid and/or further contain one or more pharmaceutically acceptable excipients. The solid load may comprise a therapeutic payload of the API for local release and/or topical treatment of a patient condition at the gastric region or for systemic absorption. The API may be any one of the APIs mentioned earlier, and/or for the treatment of any disease or medical condition mentioned earlier. The solid load may comprise no protective cover (e.g., a coating), as the device may comprise a locking container for delivery into the stomach, thereby ensuring that the load remains functional (and optionally substantially intact) until it reaches the stomach. Alternatively, the solid load may have a coating, so as to release the API only when desired. In variations, the API may be carried by the device in a semi-solid or gel form, or in (any combination of) a solid, semi-solid, powder, gel, and/or liquid form (i.e., partly in one form, and partly in one or more other forms).

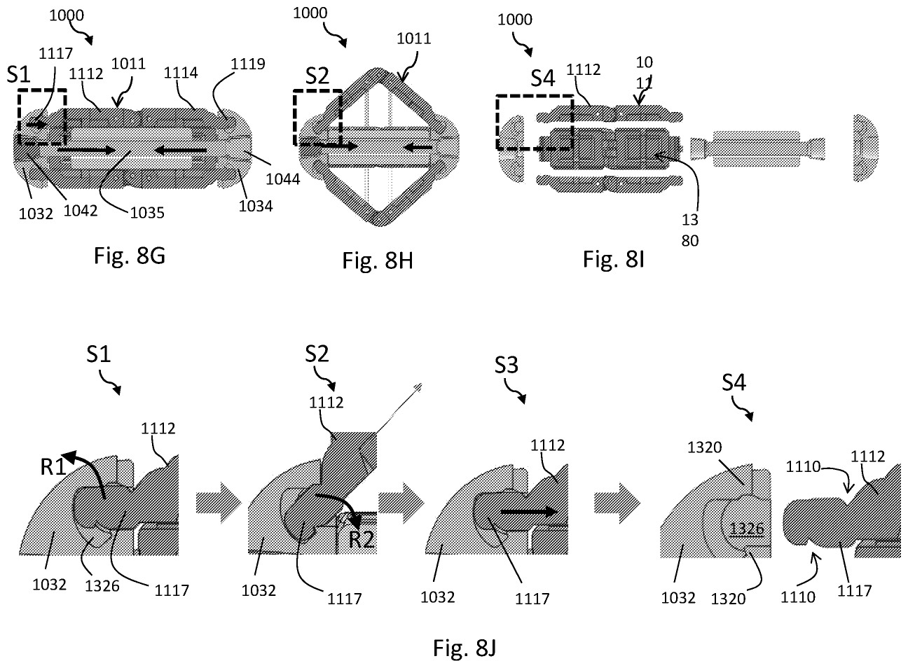

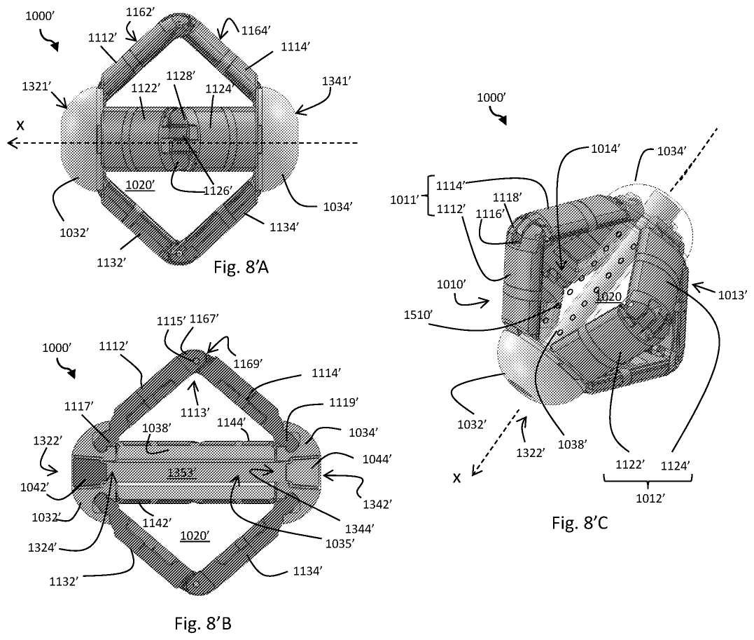

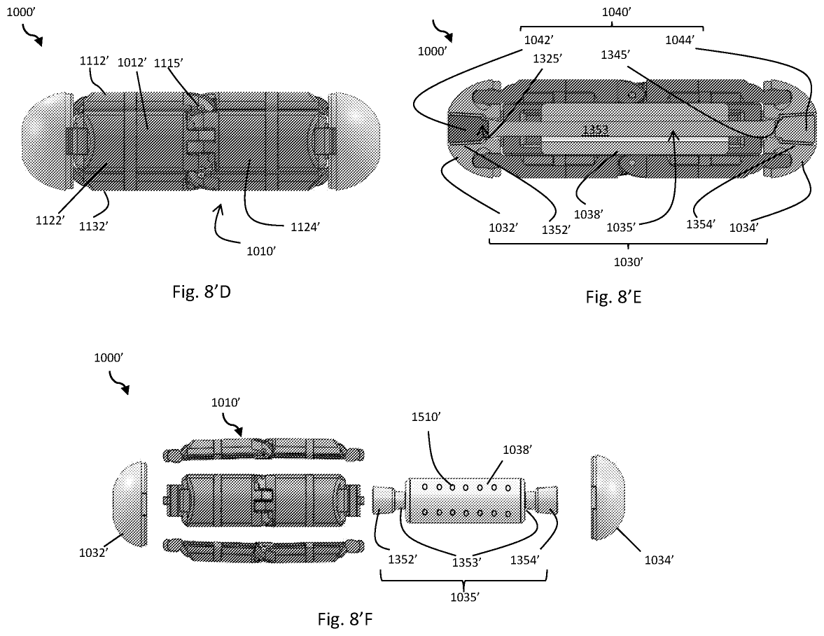

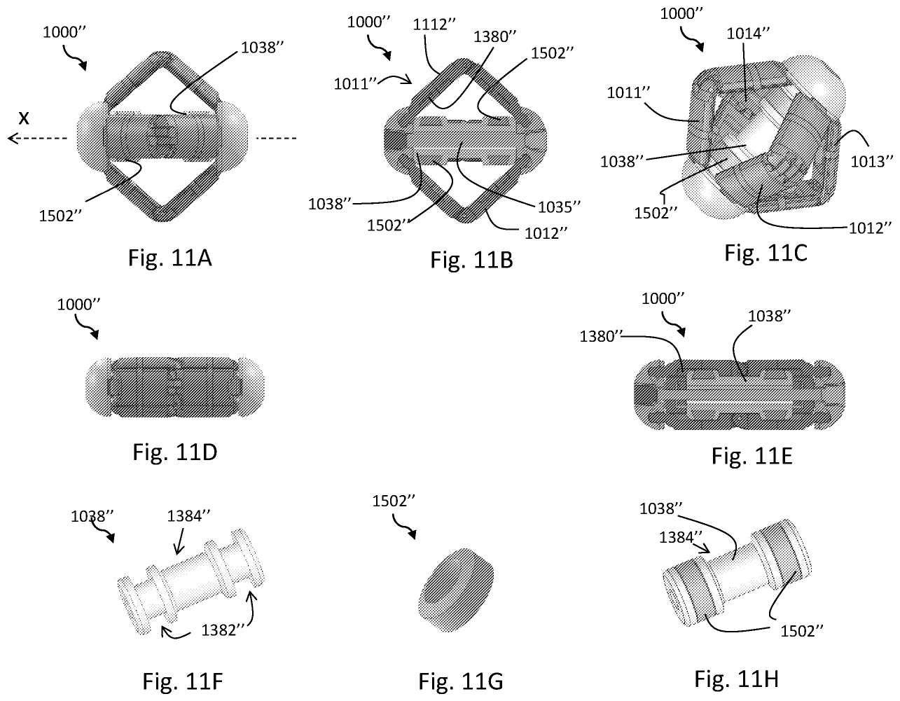

For example, the device 100 may comprise an inner space 22 in the closed configuration formed between the arms 11-14 and the elastic member 35 (see FIG. 5B), and the inner space 22 may contain a load of an API. Alternatively of additionally, the device 100 may comprise a coating on the inner surfaces 23 of the arms 11-14 (see FIGS. 2A and 5B), and the coating may contain a load of an API. Optionally, the device 100 may comprise a support tube (not shown), and the elastic member 35 may be arranged inside the support tube. The support tube may be as later described with reference to FIGS. 8A-8J and/or FIGS. 11A-11G, and notably (further optionally) carry a load of an API inside or thereon. When the device 100 expands from the closed configuration of FIG. 1B or 5A-5B into the expanded configuration of FIG. 1A or 2A-2B, the load is exposed to chyme flow through the openings 20 and thereby releases the API.

Thanks to its carrying a load of the API and its being retained in the stomach of the patient, the device 100 allows transporting and maintaining the API for release in the stomach over a desired period of time.

Following oral administration, in some embodiments, the device may be used in conjunction with an object in the form of an emptying dosage form that is configured to cause transfer of the device into the emptying configuration (i.e., to activate the trigger assembly 40). In some embodiments, as described above, the trigger assembly may be activated by the device being exposed to a predetermined set of activation environmental conditions. The emptying dosage form may be configured to cause the stomach environmental conditions to reach the activation environmental conditions. Basically, in these embodiments, an emptying dosage form for use in conjunction with the device may comprise a payload (in the form of an emptying agent) enabling to cause the environmental conditions of the stomach to reach the predetermined set of activation environmental conditions. For example, the trigger assembly may be activated when a surrounding pH reaches above a predetermined threshold (e.g., pH 6) and the emptying dosage form may be configured with a basic payload.

Following oral administration, the emptying dosage form transits along the GI until reaching the stomach where it is trapped. In the stomach, the payload of the emptying dosage form is released so as to cause the trigger assembly of the device to activate thereby causing transfer of the device into the emptying configuration.

Alternatively or additionally, the device 100 may be configured for the trigger assembly 40 to self-activate after being exposed to the stomach environment over a predetermined period of time, so as to empty the device 100 (i.e., without the use of such an emptying dosage form). In embodiments, the device forms relatively little obstruction to chyme flow, such that the device may safely remain retained in the stomach until emptying, for example after a period of at least 1.2, 1.5 or 2 times longer that the period of time required to fully diffuse the carried API.

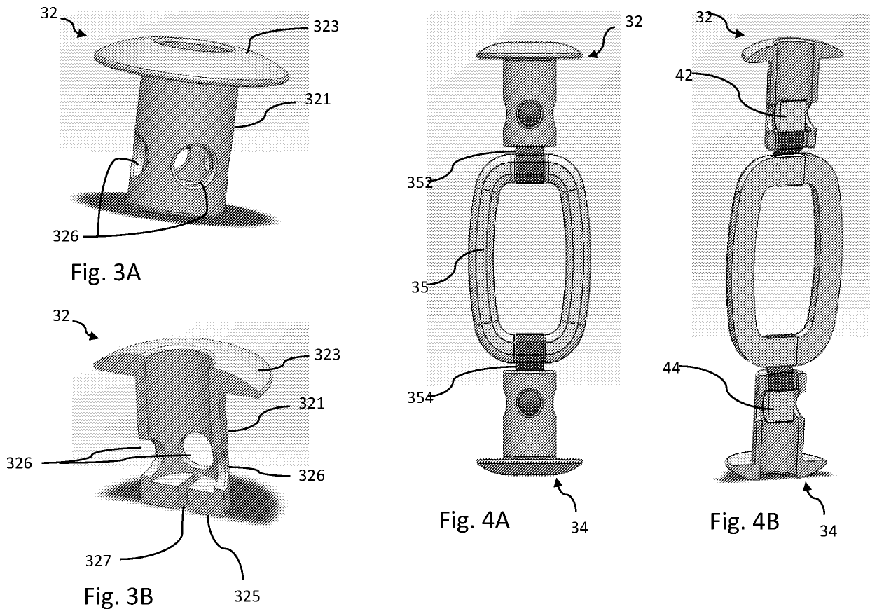

With reference to FIGS. 3A-3B and 4A-4B, more details regarding an arrangement of the biasing assembly 30 and the trigger assembly 40 of the device shown in FIG. 1 are provided.

The biasing assembly 30 may comprise two coupling heads 32, 34 and an elastic member 35. The coupling heads 32, 34 may each be arranged to sit on the four flexible arm 11-14 ends. The elastic member 35 may be arranged between the arms 11-14 and configured to force the coupling heads 32, 34 together thereby bending the arms 11-14 into the expanded configuration. In other words, each of the coupling heads 32, 34 may be configured to hook respectively four flexible arms ends and the elastic member 35 may be configured to pull the coupling heads 32, 34 together to bend the arms into a rounded shape.

The elastic member 35 may be releasably secured to the coupling heads 32, 34 by the support elements 42, 44 of the trigger assembly 40. In some embodiments, the support elements 42, 44 are erodible elements configured to degrade when the device is exposed to an activation signal, and the erosion of the support elements releases the arm from the coupling heads 32, 34 from the elastic member 35 so that the device irreversibly returns into a compact configuration similar to the closed the closed configuration in which it may be capable of being emptied through the pyloric valve. The elastic member 35 may comprise extension straps 352, 354. The extension straps 352, 354 may or not be unitary with the elastic member 35.

For the sake of conciseness, a detailed description of the arrangement of the support elements and coupling heads is given only for one of the two coupling heads 32, 34, support elements 42, 44 and extension straps 352, 354. It is understood that a similar arrangement may be foreseen for the other coupling head 34, support element 44 and extension strap 354. The coupling head 32 may include a hollow shank 321 and a head 323 forming a circumferential ledge at an upper end of the hollow shank 321. The hollow shank 321 may be positioned concentric to the four arms 11-14. The circumferential ledge of the head 323 may be seated on the arm 11-14 ends. A lower end of the shank 321 may comprise an inner plate 325 protruding radially from an inner surface of the shank 321. The inner plate 325 may include a through opening 327 which can be formed as a slot. The shank 321 may further comprise one or more piercing holes 326 configured to enable liquid entering the hollow shank 321. The support element 42 may be supported on the inner plate of the coupling head 32. The extension strap 352 may form a loop passing through the opening 327 in the inner plate 325 of the shank 321 and wrapping around the support element 42 thereby securing between the coupling head 32 and the elastic member 35.

FIGS. 5A-5B illustrate the device shown in FIG. 1 but in the emptying configuration. As can be seen, the emptying configuration can resemble the closed configuration. In some embodiments, the emptying configuration can even be identical to the closed configuration. As shown in FIG. 5A, an external envelope of the device in the emptying configuration may be identical to an external envelope of the device in the closed configuration. However, as shown in FIG. 5B, the elastic member 35 is in a relaxed state and the extension straps 352 and 354 are free. The support elements 42, 44 have degraded and are not visible on FIG. 5B since the emptying configuration follows activation of the trigger assembly.

An assembly method of a device 100 as shown on FIG. 1 is now described. In a first step, a silicone high shore (80A) tube having 30 mm length and 8 mm external diameter and 3 mm internal diameter is cut symmetrically with four central longitudinal slits of 26 mm leaving about 2 mm of each side not cut. This provides the four flexible arms 11-14 coupled to each other at both end by a top and a bottom coupling portion. Then, in a second optional step, a circular string ring made of elastic shore 40A silicone string having 0.7 mm thickness and 1.2 mm width and diameter of 10 mm is connected on the four arms of the tube at a longitudinal middle portion of the four arms. This provides for the circumferential belt 15. Connecting of the circular string to the arms can be done with silicone glue. The thickness, width and arrangement of the circumferential belt 15 and flexible arms 11-14 result in a meshwork having eight openings 20. The eight openings 20 may have a surface area of about 24.8 mm² each when the device is in the expanded configuration using the biasing assembly described hereinbelow.

The coupling heads 32, 34 are made of aluminum. An external diameter of the circumferential ledge of head 323 may be of about 8 mm. An internal diameter of the shank 321 is of about 3.5 mm. The elastic member 35 is made of an elastic band ring of 6 mm external, 4 mm internal diameter having 1 mm thickness, 3 mm width made of silicone shore 50A. In a third step, the elastic member 35 is fixed at two diametrically opposed points to extension straps 352, 354 made of a non-ductile material. The extension strap 352, 354 are then pulled through the openings 327 in the inner plates 325 of the shanks 321 of the coupling head 42, 44. Eudragit® L or S pins of 1.6 mm diameter and 2.5 mm length as support elements 42, 44 are then sandwiched between the extension straps 352, 354 and the openings 327 in such a way that upon erosion of the pins 42, 44, the extension straps 352, 354 freely detach from the coupling heads 32, 34. This provides the device in the unfolded configuration. Such four-arm spherical device in the folded configuration has dimensions enabling efficient encapsulation into a 31 mm length and 11 mm diameter capsule. Based on this principle, various alternative spherical shapes may be made, e.g., having different number of arms, arm length, materials composition (e.g., corners made of hard polymer such as polyurethane) enabling for example to tune up the device dimensions and mechanical properties to meet different targets (for example specific human population or for veterinary purposes. For example, a device made of six arms having a length of 33 mm and a circumferential belt of 1 mm width will result in meshwork having 12 holes, that can be encapsulated into a 35 mm by 11 mm capsule. Each hole may have an external surface area of about 26.7 mm² each.

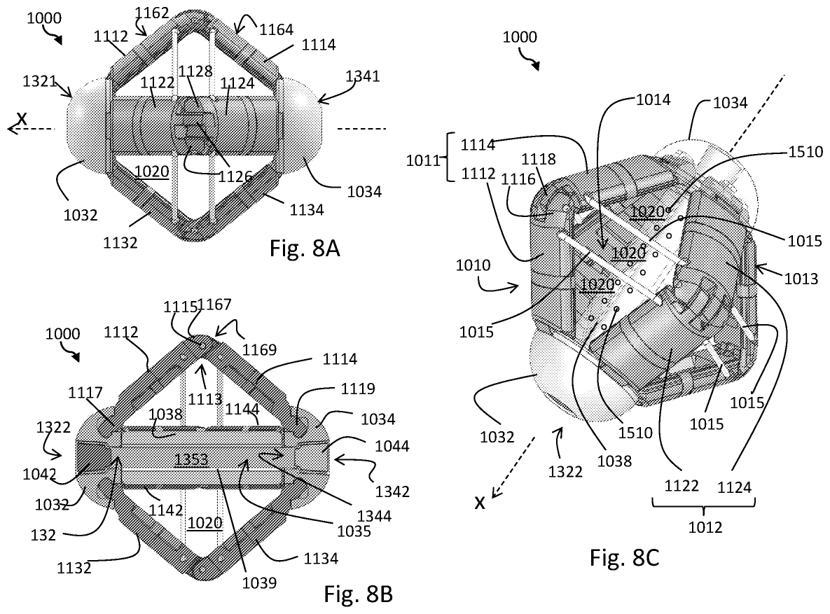

FIGS. 6A and 6B show another example of a device 100′ for temporary stomach retention according to the present disclosure respectively in an open configuration and in a closed configuration.

The device 100′ resembles the device 100 discussed above in many aspects. Accordingly, numerals used to identify features of the device 100 are appended a single quotation mark ′ to identify like features of the device 100′. It is understood that the features which are similar to device 100 and combinable with device 100′ are not all repeated in details below.

The device 100′ may comprise a body 10′, a biasing assembly 30′, a trigger assembly 40′ and optionally a padding assembly 50.

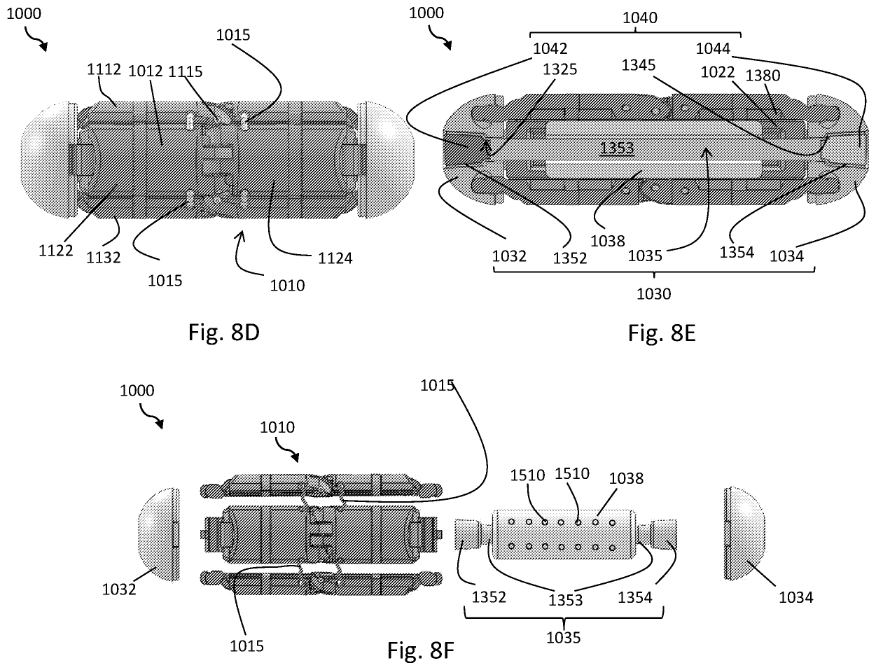

The body 10′ may comprise several (e.g., four) flexible arms and an optional circumferential belt (not shown) disposed around the flexible arms. In the open configuration, the body defines openings which enable chyme flow when the device is positioned in the stomach, thus enhanced release of a carried load of an API (not shown). As shown on FIG. 7, one or more of the flexible arms may also be composed of a set of rigid arm sections 111-113, 131-133. The set of rigid arm sections may be coupled end to end so that the arm shape can vary. For example, the rigid arm sections 111-113, 131-133 may be coupled end to end with a pivot-type coupling.

The biasing assembly 30′ may be configured to resiliently hold (bias) the device 100′ in the expanded configuration. In other words, the biasing assembly 30′ may be configured to cause the device to tend to resiliently return from the closed configuration into the expanded configuration. The biasing assembly 30′ may comprise an elastic member 35′ and coupling heads 32′, 34′. A first end of each flexible arm may be coupled to the first coupling heads 32′ and a second hand of each flexible arm may be coupled to the second coupling head 34′. The coupling heads 32′, 34′ may each be arranged to sit on the four flexible arm ends. Optionally, the arm ends and the coupling heads may be coupled by pivot pins 37 thereby forming hinged connections between the arm ends and the coupling heads. The elastic member 35′ may be arranged between the arms and configured to force the coupling heads 32′, 34′ together thereby biasing the device in the expanded configuration.

The elastic member 35′ may be releasably secured to the coupling heads 32′, 34′ by the support elements 42′, 44′ of the trigger assembly 40′. The elastic member 35′ may be configured to be irreversibly released from the coupling heads 32′, 34′ when the device 100′ is exposed to an activation signal, so that the device irreversibly returns into a compact configuration similar to the closed the closed configuration in which it may be capable of being emptied through the pyloric valve.

The optional padding assembly 50 may comprise a plurality of pads 51-56. The pads 51-56 may be disposed around the device 100′. The pads 51-56 may be made of a resilient material so as to be reversibly compressible. The pads 51-56 may be shaped as circles and/or as hollow cylinders. For example, the pads 51-56 may be made of silicone. Further, the padding assembly may be configured for lowering pressure on the patient tissue when the device 100′ is in the open configuration.

FIGS. 8A-8C, FIGS. 8D-8E, and FIG. 8F show another example of a device 1000 for temporary stomach retention according to the present disclosure, respectively in an expanded (open) configuration (FIGS. 8A-8C), in a closed (swallowing) configuration (FIGS. 8D-8E), and in a disassembled configuration which may form an emptying configuration and/or a pre-assembling configuration during manufacturing (FIG. 8F). FIGS. 8A, 8D, and 8F show a front view of the device 1000. FIG. 8C shows a perspective view of the device 1000 in the same configuration as in FIG. 8A. FIGS. 8B and 8E show a longitudinal cross-section view (along axis X) of the device 1000 in the same configuration respectively as in FIGS. 8A and 8D, the cross-section being taken in a median plane of the device 1000 parallel to the view plane of respectively FIGS. 8A and 8D.

The device 1000 resembles the device 100 discussed above in many aspects. Accordingly, numerals used to identify features of the device 100 are incremented by a factor 1000 to identify like features of the device 1000. It is understood that the features which are similar to device 100 and combinable with device 1000 are not all repeated in details below.

The device 1000 may be deformable from the closed configuration of FIGS. 8D-8E into the expanded configuration of FIGS. 8A-8C, and the device 1000 may be irreversibly disassembled from the expanded configuration of FIGS. 8A-8C into an emptying configuration identical or similar to the disassembled configuration of FIG. 8F.

The device 1000 may comprise at least three (e.g., four) articulated arms 1011-1014 arranged (e.g., longitudinally) alongside each other (e.g., along longitudinal axis X) and forming a body 1010 of the device (see FIG. 8C). An articulated arm is an elongated structure that includes at least one articulation between two sections or segments, allowing bending during which the two sections substantially maintain their shape.

Each articulated arm may be composed of a set of (e.g., two) (e.g., rigid) arm sections. For example, the articulated arm 1011 may be composed of a first arm section 1112 and a second arm section 1114, the articulated arm 1012 may be composed of a first arm section 1122 and a second arm section 1124, the articulated arm 1013 may be composed of a first arm section 1132 and a second arm section 1134, and the articulated arm 1014 may be composed of a first arm section 1142 and a second rigid section 1144.

Each arm section 1112, 1114, 1122, 1124, 1132, 1134, 1142, 1144 may comprise one or more (e.g., one or two) integrally formed components. Each such integrally formed component may be rigid, meaning that is has no or low resilience (in particular, a substantially zero resilience or a resilience significantly lower than the resilience of the resiliently deformable member 1035). Each integrally formed component may be made of any rigid material, such as plastic (e.g., a resin), metal, or ceramic. Each integrally formed component may be 3D printed or (e.g., injection) molded. This facilitates manufacturing. Each integrally formed component may be made of a biocompatible material. The biocompatible material may optionally be biodegradable, and further optionally be configured for being retained in the stomach for a predetermined time and for a partial or complete erosion/softening before emptying from the body. For example, each integrally formed component may be made of MEDIC© rigid material manufactured by Sratasys® 3D printer or for example made of cellulose acetate manufactured by molding. The rigidity of the articulated arms may provide high structural rigidity and retention capability of the device (capacity of the device to be retained in the stomach), while achieving a particularly high ratio of trapping volume relative to compactness of the closed configuration, thanks to allowed thinness of the arms. In variations, each arm section may be semi-rigid or made in a flexible material (e.g., silicone). In yet other variations, each arm section may comprise any combination of one or more rigid components, one or more semi-rigid components, and/or one or more flexible components. In further variations, the arm sections may be made in different materials one from another.

Each articulated arm 1011-1014 may have a first end (e.g., 1117) and a second end (e.g., 1119). The first end 1117 and the second end 1119 of articulated arm 1011 only are given a numeral reference on the figures (see FIG. 8B), for the sake of conciseness. Each first end (e.g., 1117) may be an end of the first arm section (e.g., 1112) of the articulated arm (e.g., 1011), while each second end (e.g., 1119) may be an end of the second arm section (e.g., 1114) of the articulated arm (e.g., 1011).

The device 1000 may comprise a first coupling head 1032 and a second coupling head 1034. The first and second coupling heads 1032 and 1034 may be substantially identical, each substantially centered on longitudinal axis X, and/or arranged substantially symmetrically one relative to another with respect to a median plane (not shown) of the device 1000 substantially perpendicular to axis X.

Each coupling head may be integrally formed and/or made of any material (identical to or different from the arm sections), for example a rigid material, such as plastic (e.g., a resin), metal, or ceramic, or a semi-rigid or flexible material. Each coupling head component may be 3D printed or (e.g., injection) molded. This facilitates manufacturing. Each coupling head may be made of a biocompatible material. The biocompatible material may optionally be biodegradable, and further optionally be configured for being retained in the stomach for a predetermined time and for a partial or complete erosion/softening before emptying from the body. For example, each coupling head may be made of MEDIC© rigid material manufactured by Stratasys© 3D printer or for example made of cellulose acetate manufactured by molding. The rigidity of the coupling heads may provide high structural rigidity of the device. Examples of materials that may be used for manufacture of the coupling heads, articulated arms include known pharmaceutical polymers such as HPMC-AS, Eudragit®, cellulose acetate (and it family), e.g. by manufacturing by injection molding, or for example other biocompatible materials, e.g. Class 6 grade, such as: PEEK (polyetheretherketone), Visijet M3 crystal and the like.

In the expanded configuration (see FIGS. 8A-8C), the first end (e.g., 1117) of each articulated arm (e.g., 1011) is coupled to (i.e., connected to, cooperating with) the first coupling head 1032, and the second end (e.g., 1119) of each articulated arm (e.g., 1011) is coupled to the second coupling head 1034. Thus, the first and second coupling heads 1032 and 1034 are configured to join the (e.g., four) articulated arms 1011-1014 to each other by both ends, such that the arms 1011-1014 are secured alongside each other in the expanded configuration.

In the closed configuration (see FIGS. 8D-8E), the first end (e.g., 1117) of each articulated arm (e.g., 1011) may also be coupled to the first coupling head 1032, and the second end (e.g., 1119) of each articulated arm (e.g., 1011) may be coupled to the second coupling head 1034. Thus, the arms 1011-1014 are secured alongside each other in the closed configuration as well.

The first end (e.g., 1117) of each articulated arm (e.g., 1011) may be rotatably coupled to the first coupling head 1032, and the second end (e.g., 1119) of each articulated arm (e.g., 1011) may be rotatably coupled to the second coupling head 1034. In other words, each articulated arm 1011-1014 may be connected to the first coupling head 1032 and/or to the second coupling head 1034 but with freedom in rotation relative thereto.

The respective arm sections 1112 and 1114, 1122 and 1124, 1132 and 1134, and 1142 and 1144 of each articulated arm 1011-1014 may further be coupled end to end with a pivot-type coupling (e.g., 1113). For each articulated arm (e.g., 1011) consisting of two arm sections (e.g., 1112 and 1114), each arm section may be coupled (e.g., rotatably) at one end to the first or second coupling head 1032 or 1034 and coupled (e.g., rotatably) at the other end to the other arm section. For example, the first arm section 1112 of the articulated arm 1011 is coupled (e.g., rotatably) at its one end 1117 (i.e., first end of the articulated arm 1011) to the first coupling head 1032 and at its other end 1167 to the other (i.e., second) arm section 1114 of the articulated arm 1011 with a pivot-type coupling 1113. Similarly, the second arm section 1114 of the articulated arm 1011 is coupled (e.g., rotatably) at its one end 1119 (i.e., second end of the articulated arm 1011) to the second coupling head 1034 and at its other end 1169 to the other (i.e., first) arm section 1112 of the articulated arm 1011 with the pivot-type coupling 1113. The pivot-type coupling 1113 of the articulated arm 1011 only is given a numeral reference on the figures (see FIG. 8B), for the sake of conciseness.

The device 1000 may thus form a (e.g., rigid) mobile (e.g., Sarrus) mechanism deformable from the closed configuration of FIGS. 8D-8E into the expanded configuration of FIGS. 8A-8C, optionally reversibly deformable between from the expanded configuration of FIGS. 8A-8C into the closed configuration of FIGS. 8D-8E, by converting relative rotations into a relative motion (e.g., a translation along axis X) between the first coupling head 1032 and the second coupling head 1034. The converted relative rotations are the relative rotation of each pair of coupled arm sections one with respect to the other, and the relative rotation of each arm section coupled to a coupling head 1032 or 1034 with respect to said head. Such motion conversion achieves bending (e.g., respectively, straightening) of the articulated arms to (e.g., reversibly) deform the device from (e.g., respectively, into) the closed configuration into (e.g., respectively, from) the expanded configuration. Indeed, the articulated arms are bent in the expanded configuration (see FIGS. 8A-8C), whereas the articulated arms are substantially straight in the closed configuration (see FIGS. 8D-8E). In examples, due to their rigidity, the rigid arm sections 1112, 1114, 1122, 1124, 1132, 1134, 1142, 1144 maintain their shape during the whole motion conversion, and thereby each present a substantially identical shape between the expanded configuration and the closed configuration. In other words, no rigid arm section is deformed during its relative rotation with another component.

The articulated arms 1011-1014 may be structurally identical one to another. The device 1000 may present a symmetry of revolution with respect to axis X. The arm sections 1112, 1114, 1122, 1124, 1132, 1134, 1142, 1144 may be arranged in one or more (e.g., two) layers between the coupled heads 1032 and 1034. When each articulated arm consists of two arm sections, the first arm sections 1112, 1122, 1132, 1142 may form a first layer while the second arm sections 1114, 1124, 1134, 1144 may form a second layer. The two layers may be separated by the median plane (not shown) of the device 1000 substantially perpendicular to axis X. In other words, pivot-type couplings (e.g., 1113) between the first arm sections 1112, 1122, 1132, 1142 and the second arm sections 1114, 1124, 1134, 1144 may all lie on said plane. The device 1000 may be symmetrical with respect to said median plane.

Each arm section 1112, 1114, 1122, 1124, 1132, 1134, 1142, 1144 may be of a generally prism shape (i.e., generally straight). The device 1000 may itself be of a generally prism (e.g., cylindrical) shape in the closed configuration (see FIG. 8D). Each arm section 1112, 1114, 1122, 1124, 1132, 1134, 1142, 1144 may maintain its shape (i.e., not be deformed) during use of the device 1000, for example during transfer from the closed configuration into the expanded configuration, and/or during transfer from the expanded configuration into the emptying configuration.

Each arm section 1112, 1114, 1122, 1124, 1132, 1134, 1142, 1144 may be substantially of a same length, optionally structurally identical. The device may thus have a generally (e.g., regular) bipyramidal shape in the expanded configuration, preferably a generally (e.g., regular) octahedral shape (i.e., when, the number of articulated arms 1011-1014 is exactly four). Each arm section may correspond to an edge of the bipyramidal shape, while each pivot-type coupling (e.g., 1113) and each coupling head 1032 and 1034 may correspond to a vertex of the bipyramidal shape. The device 1000 is thus configured to (e.g., reversibly) switch the generally compact prism/straightened shape of the closed configuration (with a relatively small volume occupancy) into the generally hollow bipyramidal shape of the expanded configuration (with a relatively large volume occupancy). Components of the device 1000 may be rounded at edges and/or vertices of the bipyramidal (e.g., octahedral) shape, such that the device 1000 may present in the (unfolded) expanded configuration a generally spherical convex hull 3D structure.

The rigidity of the articulated arms 1011-1014 allows the device 1000 to present a relatively high structural rigidity while making it easy to achieve an optimize use of space. In particular, a 3D structure with a high sphericity (e.g., above 0.8, e.g., measured by the Hakon Wadell sphericity equation of the convex hull of an object) and/or a low ratio between a maximal circumference and a minimal circumference (e.g., below 1.5, e.g., each circumference being the length of a planar curve traced on a periphery of an object) allows a high retention in the stomach, while enabling optimal tissue pressure distribution, e.g., when device is in the expanded configuration, and small size when in closed configuration to enable swallowing.

In examples, the volume occupied by the device in the expanded configuration may be larger than a sphere of a diameter of 17 mm, and optionally larger than a sphere of a diameter of 20 mm. In examples, the assembly consisting of the articulated arms 1011-1014 and the coupling heads 1032, 1034 in the closed configuration may present dimensions (also referred to as “folded dimensions” or “dimensions of the device when folded” or the like), such that it may be capable of being fitted into a cylinder of less than about 35 mm and of less than about 12 mm diameter. In particular, said assembly in the closed configuration may be capable of being fitted into a cylinder of length less than 32 mm, 31 mm, 30 mm, or 29 mm, and/or of diameter equal to or less than 11 mm, 10.5 mm or 10.2 mm, for example a cylinder of length less than 29 mm and/or of diameter less than 10.2 mm.

Thus, an optional (e.g., particularly easy to swallow) container (configured to degrade in stomach environmental conditions not shown on the figures) configured to temporarily at least partially enclose the device in the closed configuration (e.g., a capsule), may present a length of about 35 mm or less and/or of diameter of about 12 mm or less, for example a length equal to or less than 32 mm, 30 mm or 29 mm and/or a diameter equal to or less than 11, 10.5 mm or 10.2 mm, such as length equal to 29 mm and a diameter equal to 10.2 mm.

Such a container may form a locking element and/or be resistant to standard gastric environmental conditions and configured to degrade in stomach environmental conditions. In variations, the container may merely form a capsule to enable swallowing, and/or the device may comprise another and separate component forming such a locking element arranged to temporary maintain the device in the closed configuration for facilitating administration and/or at least partially made of a material degrading at standard stomach environmental conditions.

Yet, even with such dimensioning, the device 1000 may present sufficient available space to carry a load of an API.

Further, even with such dimensioning, the dimensions of the device in the expanded configuration may be such that the device cannot pass through an orifice of about 17 mm diameter and preferably of about 20 mm and even achieves high retention. Generally, the device 1000 may have a shape and/or a size and/or a structural rigidity enabling the device to be retained in the stomach. The capability of the device 1000 to be retained in the stomach may be defined in accordance with methods described in details herein below with reference to FIG. 12.

In embodiments, each pivot-type coupling (e.g., 1113) may comprise, for each pair of arm sections coupled end to end (by said pivot-type coupling), transversal hinge holes of the arm sections of the pair and a hinge pin passing through the transversal hinge holes. The hinge pin may be made of any rigid material, such as any plastic or any metal or any rigid biocompatible material. The hinge pin may be made, e.g., of nylon or Nitinol. The hinge pin may alternatively be made of an erodible polymer, so as to enable disassembly of the arms upon its erosion to facilitate emptying of the device after retention in the stomach. For example, the pivot-type coupling 1113 may comprise a hinge pin 1115 (see FIG. 8B) passing through both transversal hinge holes (no numeral reference) formed on the end 1167 of the first arm section 1112 and transversal hinge holes (no numeral reference either) formed on the end 1169 of the second arm section 1114. In examples, the end 1167 of the first arm section 1112 may comprise a first set of one or more (e.g., two) fingers 1126 and the end 1169 of the second arm section 1114 may comprise a second set of one or more (e.g., two) fingers 1128 (see FIGS. 8A and 8C). The fingers 1126 and 1128 may extend longitudinally from their respective arm sections and be dimensioned for the first and second sets of fingers to engage one with another, thereby reaching alignment of all the transversal hinge holes, thus allowing passage of the hinge pin 1115.

The device 1000 may optionally comprise one or more (e.g., one or two) circumferential threads 1015 circumferentially linking the articulated arms (see FIGS. 8C, 8D and 8F). Each thread 1015 may be made of an elastic or a flexible material so as to allow the articulated arms 1011-1014 to open into the expanded configuration without substantial restriction. For example, each thread 1015 may be made of silicone or fabric, synthetic polymer such as nylon, polyurethane. Each thread 1015 may pass through transversal threading holes (no numeral reference) formed on the articulated arms. Each thread 1015 may be pulled through transversal threading holes and tied at the end. The transversal threading holes may comprise one or more (e.g., one or two) layers of transversal threading holes, each layer being on a respective plane perpendicular to axis X. For example, each first arm section 1112, 1122, 1132, 1142 may comprise a respective transversal threading hole forming together a first layer of transversal threading holes perpendicular to the axis X, and each second arm section 1114, 1124, 1134, 1144 may comprise a respective transversal threading hole forming together a second layer of transversal threading holes perpendicular to the axis X (thus parallel to the first layer of transversal threading holes). A same thread 1015 may be pulled through each respective layer of transversal threading holes and tied at the end. Optionally, a same unique thread may pass through the optional different layers, or alternatively one single thread may be pulled per layer. In variations, the device 1000 may comprise one or more (e.g., one) circumferential belts instead of, or in addition to, the one or more circumferential threads. In variations, a thread 1015 may be pulled through the hinge holes of the pivot-type couplings (e.g., 1113) and serve for both circumferentially linking together the articulated arms and as a hinge connector (i.e., for achieving the hinge mechanism of the pivot-type couplings). In other words, the thread serves as both a belt and in place of a hinge pin.

The body 1010 may form the frame of the device 1000. The device 1000 may be configured to bend the articulated arms 1011-1014 in an angular shape (in the expanded configuration). When not bent, the arms 1011-1014 may substantially extend in the direction of the longitudinal axis X.

When the device 1000 is in the open (expanded) configuration, the body 1010 and the one or more (e.g., one or two) circumferential threads 1015 may form a tridimensional meshed structure. The openings 1020 formed between the articulated arms 1011-1014 and the circumferential threads 1015 in the open configuration may define a mesh of said meshed structure. The openings 1020 may be configured to enable chyme flow.

The circumferential threads 1015 may have a structural function, for example stabilize the platform formed by the device 1000 in the expanded configuration. This improves stomach retention.

The device 1000 in the expanded configuration has an external envelope configured to contact the subject tissue (see FIG. 8A). The external envelope comprises the external surface 1321 of the first coupling head 1032, the external surface 1341 of the second coupling head 1034, and the external surface (e.g., 1162, 1164) of each articulated arm (e.g., 1011). The external surfaces 1162, 1164 of the first and second arm sections 1112, 1114 of articulated arm 1011 only are given a numeral reference on the figures, for the sake of conciseness. The external envelope of the device 1000 may be blunt to avoid damaging the subject tissue. In particular, the coupling heads 1032, 1034 may be made of a smooth material (e.g., plastic, such as resin, or polyurethane, or other biocompatible material) and/or of an externally smooth (e.g., rounded) shape, for example of a general shape of a section of a sphere or ovoid (e.g., semi-sphere or semi-ovoid). Also, the articulated arms 1011-1014 may be made of a smooth material (e.g., plastic, such as resin) and/or of an externally smooth shape, for example of a general shape of a longitudinal section of a cylinder or a prism having an elliptical base. In addition, the external surface (e.g., 1162, 1164) of the ends (e.g., 1167, 1169) of the arm section (e.g., 1112, 1114), for example the external surface of the fingers 1126 and 1128, may be longitudinally rounded. Thus, even when they are bent, the articulated arms 1011 present a smooth vertex at the pivot-type coupling region between the arm sections, to avoid damaging tissue.

The device 1000 may further comprise a resiliently deformable (biasing) member 1035 arranged between the articulated arms 1011-1014 and configured to force the coupling heads 1032 and 1034 together to bend the arms thereby biasing the device in the expanded configuration. Thus, the coupling heads 1032 and 1034 come close one to another in the trapping configuration thanks to retraction of the resiliently deformable member 1035 (i.e., the coupling heads 1032 and 1034 are closer one to another in the trapping configuration than in the closed configuration). The resiliently deformable member 1035 may further be arranged inside an optional support tube 1038. The coupling heads 1032 and 1034 may thus form, with the resiliently deformable member 1035 and with the optional support tube 1038, a biasing assembly 1030 configured to resiliently hold (bias) the device 100 in the expanded configuration. In other words, the biasing assembly 30 may be configured to cause the device to tend to resiliently return (unfold) from the closed configuration into the expanded configuration.

The resiliently deformable member 1035 may be configured to be stretched (along longitudinal axis X, into the closed configuration). For example, the resiliently deformable member 1035 may be elastic (i.e., made of an elastic material, such as silicone). For example, the resiliently deformable member 1035 may be made of silicone shore 55A, for example as a section of a silicone tube having a cross section area of about 3 mm². In variations, the resiliently deformable member may be a spring, for example made of metal (such as stainless steel or Nitinol).

The resiliently deformable member 1035 may be secured to both the first and second coupling heads 1032 and 1034. The resiliently deformable member 1035 may comprise a first extension 1352 and a second extension 1354 at its extremities (i.e., edges), and a central resiliently deformable portion 1353 therebetween (see FIG. 8F). The first extension 1352 may be secured to the first coupling heads 1032, and the second extension 1354 may be secured to the second coupling heads 1034. The first and second extensions 1352, 1354 may or not be unitary with the central resiliently deformable portion 1353. In the latter case, the central resiliently deformable portion 1353 may be secured at its two extremities to the first and second extensions 1352, 1354. The first and second extensions 1352, 1354 may or not be resiliently deformable. The first and second extensions 1352, 1354 may form extension straps.

The first coupling head 1032 may comprise a first longitudinal hollow portion formed along axis X between a first external opening 1322 and a first base opening 1324, and/or the second coupling head 1034 may comprise a second longitudinal hollow portion formed along axis X between a second external opening 1342 and a second base opening 1344 (see FIG. 8B). The resiliently deformable member 1035 may be configured to have the first and second extensions 1352 and 1354 inserted and secured into the first and second hollow portions, for example by the first and second extensions 1352 and 1354 being pulled through the first and second base opening 1324 and 1344 and then secured in the first and second hollow portions.

The resiliently deformable member 1035 may be secured to at least one (e.g., only one, or alternatively, both) of the first and second coupling heads 1032 and 1034 releasably. When the resiliently deformable member 1035 is released from the first coupling head 1032 and/or from the first coupling head 1034, the resiliently deformable member 1035 stops to force the coupling heads together 1032 and 1034. Thus, the resiliently deformable member 1035 stops forcing the bending of the articulated arms 1011-1014. This causes transfer from the expanded configuration into an emptying configuration, the device being thereby configured to pass through the pyloric valve.

The support tube 1038 may be a hollow tube made of any material, such as a rigid material (e.g., plastic, metal, ceramic) or an elastic material.

The support tube 1038 may comprise apertures 1510 formed on a peripheral wall thereof (see FIGS. 8C and 8F). The apertures 1510 provide exposure to an interstice 1039 formed between the support tube 1038 and the resiliently deformable portion 1353 of the resiliently deformable member 1035g (see FIG. 8B). The device 1000 may accommodate a load of API (not shown) in said interstice 1039. The apertures 1510 enable to put said API in fluid communication with chyme flow, thereby enabling release/diffusion of the API carried inside the support tube 1038. The API may be carried inside the support tube 1038 in any manner, for example in a solid (e.g., pellets), semi-solid, powder, gel, and/or liquid form. The apertures may present a design which provides a predetermined release rate. Alternatively or additionally, the exposure apertures may be coated, so as to expose the contained API load only when desired, or on the contrary uncoated.

In addition, the surrounding tissue (e.g., in stomach) may press (compress) the device 1000. Such pressure when applied from the X-axis on the external surface of head 1032 and head 1034 inwards might move the two heads towards each other. A significant movement would result in deformation of the device's structure and even collapse. The support tube 1038 may resist this collapse and maintain the device unfolded. When made of material having elastic properties, the support tube 1038 may have a safety shock absorption element enabling partial limited movement of the heads towards each other. This enables the device to be compliant with the tissue, e.g., when high pressure applied by tissue, e.g., in abnormal conditions. For example, the support tube 1038 may be made of silicone shore 55A, for example as a section of a silicone tube having an external diameter of about 4 mm and an internal diameter of about 2 mm. The support tube 1038 may present a length corresponding to that of a maximal authorized diameter for the device 1000 in the expanded configuration. The support tube 1038 thus allows maintaining a minimal distance (corresponding to the length of the support tube 1038) between the two coupling heads 1032 and 1034, thereby preventing the device 1000 from expanding beyond the maximal authorized diameter (e.g., in the Y axis when pressure is applied from the X axis on the two coupling heads). This improves safety of the device 1000, by reducing risks of tissue damage.

Alternatively or additionally, the device 1000 may carry a load of API not shown) free inside an inner space 1022 formed in the closed configuration around the resiliently deformable member 1035 and delimited by the arms 1011-1014 and the coupling heads 1032, 1034 (see FIG. 8E). The API may be carried inside the inner space 1022 in any manner, for example in a solid (e.g., pellets), semi-solid, powder, gel, and/or liquid form.

Alternatively or additionally, the device may comprise an exposed recess 1380 formed on an internal surface of each arm section (see FIGS. 8E and 8I). The exposed recess may lodge a load of API (not shown), for example filling the recess. The API may be lodged inside the recess 1380 in any manner, for example in a solid (e.g., pellets), semi-solid, powder, gel, and/or liquid form.

Alternatively to such materials, the support tube 1038 and/or the arms 1011-1014 may be at least partially made or coated with a material composed at least in part of the API. As the support tube 1038 and the arms 1011-1014 are exposed to chyme flow, the material may diffuse the API as it erodes.

The device 1000 may comprise a trigger assembly 1040 (see FIG. 8E), forming an opening assembly with the biasing assembly 1030. The trigger assembly 1040 may be configured, when the device in the expanded configuration is exposed to any respective activation signal from a predetermined set of one or more activation signals, to trigger release of the resiliently deformable member 1035 from the first and/or second coupling heads 1032, 1034. The device 1000 may be configured to transfer into an emptying configuration in which it can pass through the pyloric valve under standard GI motility conditions. In some embodiments, the capability of the device 1000 to pass through the pyloric valve in the emptying configuration may be defined in accordance with methods described in details herein below with reference to FIG. 12.

The trigger assembly 1040 may include a first support element 1042 configured for securing the first extension 1352 to the first coupling head 1032, and/or a second support element 1042 configured for securing the second extension 1354 to the second coupling head 1034.

In some embodiments, the support elements 1042, 1044 may have a pin shape. The first and second extensions 1352, 1354 may for example be shaped as socks in which the support elements 1042, 1044 can be inserted via the first and second external openings 1322, 1342. The pin-shaped support elements 1042, 1044 may be inserted in the first and second extensions 1352, 1354 while press-fitted inside the first and second hollow portions (of the first and second coupling heads 1032, 1034). The press-fitting may maintain the first and second extensions 1352, 1354 securely attached to the first and second coupling heads 1032, 1034. Alternatively or additionally, the first and second hollow portions of the first and second coupling heads 1032, 1034 may comprise locking corners 1325, 1345, that cooperate with an extremal portion of the pins-shaped support elements 1042, 1044, to locally increase the press-fitting force and improve retention of the resiliently deformable member 1035 by the support elements 1042, 1044.

In some embodiments, the support elements 1042, 1044 may be erodible elements configured to degrade when the device is exposed to an activation signal, and the erosion of the support elements releases the coupling heads 1032, 1034 from the resiliently deformable member 1035 so that the device is irreversibly allowed to go into the emptying configuration in which it may be capable of being emptied through the pyloric valve. The support elements 1042, 1044 may be erodible layer-by-layer starting from the first and second external openings 1322, 1342, which are exposed to the environment of the device 1000, and going toward the first and second base openings 1324, 1344. As long as there remains at least a layer of a support element 1042 or 1044 with a diameter higher than a diameter of the first or second base opening 1324 or 1344, the support element 1042 or 1044 maintains its supporting function. As soon as the erosion reaches a (triggering) point where no such layer remains in the support element 1042 and/or 1044, the resiliently deformable member 1035 is free to retract and detach from the respective coupling head 1032 and/or 1034. Such a triggering point may be said to activate the trigger assembly 1040, and each support element 1042, 1044 may be referred to as a “timer”.

Thanks to the first and second external openings 1322, 1342 being formed on top of the coupling heads 1032, 1034 (i.e., opposite to the base of the coupling heads 1032, 1034 where the articulated arms are coupled), the first and second external openings 1322, 1342 are oriented longitudinally (on axis X), with no element of the device 1000 forming an obstruction. This arrangement allows a particularly fine control of the support element or timers 1042, 1044, thereby,-when at least one of the timers 1042, 1044 is triggered, the deformable resiliently deformable member 1035 may shrink backwards, thus releasing 1354 from the corresponding head openings 1322 or 1342 and the device unfolds.

Pin-shaped support elements 1042, 1044 may be made of any material and/or manufactured as described in reference to the device 100.

For example, pin-shaped support elements 1042, 1044 may be made of Eudragit® L or S may be manufactured as follows: a Hot Melt Extrusion machine is set to for example about 130° C. Eudragit® L or S powder is fed into the HME machine for example by a gravimetric feeder at a rate of 1 kg/hr. The HME machine snail speed is set to 100 rpm. The melted material is drawn from the HME machine, it is forwarded as strands onto a conveyor belt to cool. Once cooled, the strand is chopped by a chopping machine to a pin shape of about 1.5 mm diameter and 2 mm length. An activation signal increasing the pH in the stomach may be used to erode the pin to disassembly the device.

For example, the support elements 1042, 1044 may be configured to be disabled after the device 1000 is exposed to standard stomach environmental conditions for a predetermined residence time period, for example of one day, two days, three days or more and/or twelve weeks or less (e.g., any time period from 1 to 12 weeks, such as one month, one week, two weeks, three weeks, or even one day or two days). In other words, the trigger assembly 1030 may activate after the device is positioned in the stomach for a predetermined time period or after a predetermined time period elapsed subsequent to swallowing of the device.

The support elements 1042, 1044 may also or alternatively be configured to be disabled after the device is exposed to a predetermined set of activation environmental conditions. For example, the support elements 1042, 1044 may be configured to be disabled when a surrounding environment reaches (e.g., above) a predetermined pH threshold (e.g., pH 6, 7 or 8). For this purpose, the support elements 1042, 1044 may be erodible elements at least partially made of a material configured to dissolve (or degrade) when the surrounding pH reaches the predetermined pH threshold. In some embodiments the support elements 1042, 1044 may be at least partially made of a material dissolving at (i.e., when the pH reaches) the predetermined pH threshold. For example, the support elements may be made of a base-soluble polymer. For example, the support elements 1042, 1044 may be at least partially made of a material soluble at of a material soluble at pH above 6 such as Eudragit® L or of a material soluble at pH above 7 such as Eudragit® S).

In some embodiments, the support elements 1042, 1044 may be at least partially made of a material dissolving in standard stomach region environmental conditions, such as EUDPRAGIT EPO, and coated with a material dissolving at the predetermined pH threshold, such as Eudragit® L or S. Such pin-shaped coated support elements may be manufactured as follows: The Hot melt extrusion machine (HIME) is preheated to for example about 120-150° C. The HME machine snail speed is set to 100 rpm. Powder of Eudragit® EPO is added into HME feeder and pushed by the snail outwards. As the melted material is drawn from the HME machine, it is forwarded as strands onto a cooling machine to cool on a conveyor belt. Once cooled, the strand is chopped by a chopping machine to pin shape of about 1.5 mm diameter and 2 mm length. The pins are then placed in a coating system (e.g., vector coater) and coated with Eudragit® L or S coating with a total of for example about 10% of weight gain.

The support elements 1042, 1044 may be arranged to form keystones of the device in the expanded configuration so as to provoke a collapse of the device into the emptying configuration after the device is exposed to the predetermined activation signal. In other words, the support elements 1042, 1044 are arranged to form one or more structural weak points. When the support elements 1042, 1044 are disabled, the one or more structural weak points cause the device to fall apart in the emptying configuration. The support elements 1042, 1044 are arranged so that a transfer duration of the device from the expanded configuration into the emptying configuration is substantially smaller (e.g., equal or less than ½, ⅓, ¼, ⅕, ⅙, 1/7, ⅛, 1/9, 1/10, 1/100, 1/1000) than a standard residence time period (e.g., equal or more than 1, 2, 3, 4, 5, 6, 7 or 8 weeks). This provides an improved control of the device in in-vivo conditions. In particular, in embodiments in which the trigger assembly is activated by external means, this enables to quickly empty the device on-demand without requiring an endoscopy procedure even in urgent cases.

In embodiments, the support elements 1042, 1044 may be configured to be disabled upon the device in the expanded configuration being exposed to a different activation signal. This provides flexibility in the manner of transferring the device 1000 into the emptying configuration. For example, the support elements 1042, 1044 may be made of different materials. For example, the support element 1042 may be made of a type A material, while the support element 1044 may be made of a type B material.

In specific implementations of the timer, the type A timer 1042 may be made of IPMC AS HG:MG:DBS ratio 8:2:1 by hot-melt extrusion. This 3-component composition includes HPMC-AS high grade (HG) soluble in pH above 7: HPMC-AS medium grade (MG) soluble in pH above 6.5 and DBS which is the plasticizer dibutyl sebacate. HPMC-AS HG and HPMC-AS MG powders may be placed in a (e.g., DIOSNA) mixer and mixed together. Then while mixing, a DBS solution may be added. After mixing, the granulate may be emptied from the mixer, transit through hot melt extrusion conveyed through a cooling system and chopped to pin beads. The type B timer 1044 may be made of Eudragit® EPO by hot melt extrusion. The powder may transit through hot melt extrusion conveyed through a cooling system and chopped to pin beads. The chopped beads can be optionally be used as a raw material for injection molding to manufacture specific pin mold designs. Type A timer may be activated by an external activation signal that increases the internal gastric pH, e.g., by a basifying agent, in case of emergency, while type B timer may be designed to be activated by natural gastric surrounding environment without any external signal being activated, e.g., by gradually erosion of the type B pin. The erosion rate of Type B timer may, e.g., be controlled by adding any biocompatible and or pharmaceutical material that may control the release of the timer, that can be for example added either in the timer matrix or as a coat or alike. In other words, in some embodiments, support elements configured to be disabled after the device is exposed to standard stomach region environmental conditions for a predetermined residence time period may be made of a combination of a material degrading at standard stomach environmental conditions (such as Eudragit® EPO) and a control release delay material (such as a polymer carrier).

The device 1000 may be configured for the disassembling of the body 1010, formed by the articulated arms, from the first and second coupling heads 1032, 1034 upon activation of the trigger assembly 1040 (see FIG. 8F). Such exploding of the device 1000 from the expanded configuration into the emptying configuration facilitates the emptying when the trigger assembly 1040 activates, thus increasing safety of use. FIG. 8F shows an emptying configuration where not only the body 1010 is disassembled from both the first and second coupling heads 1032, 1034, but also the resiliently deformable member 1035 is disassembled from both the first and second coupling heads 1032, 1034. This is because both support elements 1042, 1044 have been degraded to an activation point. In embodiments, the device 1000 may achieve one or more other emptying configurations. For example, the resiliently deformable member 1035 may remain attached to one of the coupling heads 1032, 1034 and detach only from the other one, when only one of the support elements 1042, 1044 has been degraded to the activation point. This is likely to occur when different support elements/timers 1042, 1044 configured to degrade under different activations signals are used. Still, the body 1010 is disassembled from both the first and second coupling heads 1032, and this may be enough for a secure emptying.

In reference to FIGS. 8G-8J, the disassembling of the body 1010 from both the first and second coupling heads 1032 is now discussed in more details.

FIGS. 8G-8I show the device 1000 respectively in the closed, expanded, and emptying configuration, corresponding to a standard sequence of use of the device 1000. FIG. 8J further illustrates the sequence of use by showing close-up views of a zone S1-S4 of the device where the first arm section 1112 of the articulated arm 1011 is coupled to the first coupling head 1032.

As shown, the first end (e.g., 1117) of each articulated arm (e.g., 1011) is coupled to the first coupling head 1032 releasably, and the second end (e.g., 1119) of each articulated arm (e.g., 1011) is coupled to the second coupling head 1034 releasably. In the expanded configuration (see FIG. 8H), said first end 1117 is inserted in a respective cavity 1326 of the first coupling head 1032 (see FIG. 8J, last step, i.e., close-up S4), and said second end 1119 is inserted in a respective cavity of the second coupling head 1034 (not shown). Indeed, the resiliently deformable member 1035 maintains said first end and second end 1117 and 1119 each secured inside the respective cavities. This is thanks to the resiliently deformable member 1035 being securely attached to the first and second coupling heads 1032-1034 by the support elements 1042, 1044, thus pulling the first and second coupling heads 1032-1034 together when transferring from the closed configuration (FIG. 8G) into the expanded configuration (FIG. 8H), thereby exerting a pressure maintaining the articulated arms 1012-1014 therebetween in the expanded configuration. The retraction of the resiliently deformable member 1035 and the movement of the first and second coupling heads 1032-1034 one toward the other is represented by arrows on FIGS. 8G-8H.

The respective cavity 1326 of the first coupling head 1032 may be dedicated to the coupling of the articulated arm 1011, and each other articulated arm 1012-1014 may cooperate with a different other cavity of the first coupling head 1032. Such arm head cooperation increases the stability of the assembled device when in open unfolded configuration while exposed to external forces (e.g., when a force is applied on arm couplings, e.g., 1113) Similarly, the second coupling head 1034 may comprise a single cavity per articulated arm 1011-1014. Alternatively, any or both the first and second coupling heads 1032 and 1034 may comprise a circumferential cavity (e.g., 1326) configured to receive a respective end of each articulated arm 1011-1014.

Referring to FIG. 8J in particular, the discussion now focuses on the coupling and release of the first arm section 1112 of the articulated arm 1011 with respect to the first coupling head 1032. But the discussed coupling and release may identically apply to the first arm section of each other articulated arm 1012-1014 with respect to the first coupling head 1032, and/or to the second arm section (e.g., 1114) of each articulated arm 1011-1014 with respect to the second coupling head 1034.

The first end 1117 (and thus the first arm section 1112) may be rotatable in the respective cavity 1326. Thus, when the device 1000 transfers from the closed configuration (close-up S1) into the expanded configuration (close-up S2), the first end 1117 makes a rotation R1 relative to the first coupling head 1032, due the resiliently deformable member 1035 pulling the coupling heads 1032 and 1034 together. Similarly, when the device 1000 transfers from the expanded configuration (close-up S2) into the emptying configuration, the first end 1117 initially makes an inverse rotation R2 relative to the first coupling head 1032 (close-up S3), due to the resiliently deformable member 1035 being detached and thus stopping exerting its force, thus allowing the GI mechanical forces to apply on the device 1110 (e.g., due to chyme flow and tissue-induced pressures), thereby straightening the articulated arm 1011. Afterwards, when the device continues to transfer from the expanded configuration into the emptying configuration, the first end 1117 may be dimensioned to move out of the respective cavity 1326, due again to the GI mechanical forces. Thus, the first arm section 1112 and the first coupling head 1032 form a hinge disassembly mechanism.

As best seen on close-up S2, the first arm section 1112 may transit in an angle to the first coupling head 1032 and then be locked in head. The first coupling head 1032 may comprise for that a stopper mechanism. The stopper mechanism may comprise dents 1320 of the first coupling head 1032 cooperating with recesses 1110 of the first end 1117 (see close-up S4). Surfaces forming the recesses 1110 may engage surfaces forming the dents 1320, thereby preventing disassembly of the arm from the coupling head in the expanded configuration, even if GI mechanical forces increase.

In embodiments, in the folded configuration, the arm and head are orientated in a same axis while the arm is forced forward into the head cavity by the biasing member. During unfolding, the arm transits in an opposite angle to the head, and this positions the arm in a locking position within the head. After the timer is released, the biasing member is detached, the arm returns in a backward movement back into the folding position. As the biasing member is now detached, the arm is no longer forced into the head, and thus can be disassembled from the head when a mild external force is applied.

In specific implementations of the device 1000, preparation and assembly of the rigid device may be performed as follows. Device arms and may be made by Stratasys® 3D printer rigid VERO or MEDIC medical grade material. Each two arms may be hinged together with a nylon pin. The biasing member may be made out of a silicone tube 55A shore of about 3 mm² cross section may be pulled through one of the heads. The first end may be locked by a timer of type A (made of [[HPMC AS HG:MG:DBS ratio 8:2:1 by hot-melt extrusion). The second end may be pulled through a support tube made out of a silicone 55a shore tube of 4 mm external diameter and 2 mm internal, and then through the second head and locked by a timer of type B (made of Eudragit® L or S by hot-melt extrusion). The arms may be then hinged to the heads via the above-illustrated arm-head locking structure. A circumferential thread may be pulled through designated threading holes in the arms, and tied at the end.

FIGS. 8′A-8′C, FIGS. 8′D-8′E, and FIG. 8′F show another example of a device 1000′ for temporary stomach retention according to the present disclosure, respectively in an expanded (open) configuration (FIGS. 8′A-8′C), in a closed (swallowing) configuration (FIGS. 8′D-8′E), and in a disassembled configuration which may form an emptying configuration and/or a pre-assembling configuration during manufacturing (FIG. 8′F). FIGS. 8′A, 8′D, and 8′F show a front view of the device 1000′. FIG. 8′C shows a perspective view of the device 2000 in the same configuration as in FIG. 8′A. FIGS. 8′B and 8′E show a longitudinal cross-section view (along axis X) of the device 1000′ in the same configuration respectively as in FIGS. 8′A and 8′D, the cross-section being taken in a median plane of the device 1000′ parallel to the view plane of respectively FIGS. 8′A and 8′D.

The device 1000′ resembles the device 1000 discussed above in many aspects. Accordingly, numerals used to identify features of the device 1000 are appended a single quotation mark ′ to identify like features of the device 1000′. It is understood that the features which are similar to device 1000 and combinable with device 1000′ are not all repeated in details below.

The device 1000′ mainly differs from the device 1000 in that it comprises no circumferential thread 1015. The device 1000′ may allow particularly non-obstructed chyme flow, thus allowing long but safe retention of the device at the stomach. Thus, the device 1000′ may be configured for self-emptying from the stomach after a predetermined period of time in safety. Yet, the device may be sufficiently rigid even without any circumferential thread or belt.

For example, the support elements 1042′, 1044′ may be configured to be disabled after the device 1000′ is exposed to standard stomach environmental conditions for a predetermined residence time period, for example of one day, two days, three days or more and/or twelve weeks or less (e.g., any time period from 1 to 12 weeks, such as one month, one week, two weeks, three weeks, or even one day or two days), as previously discussed.

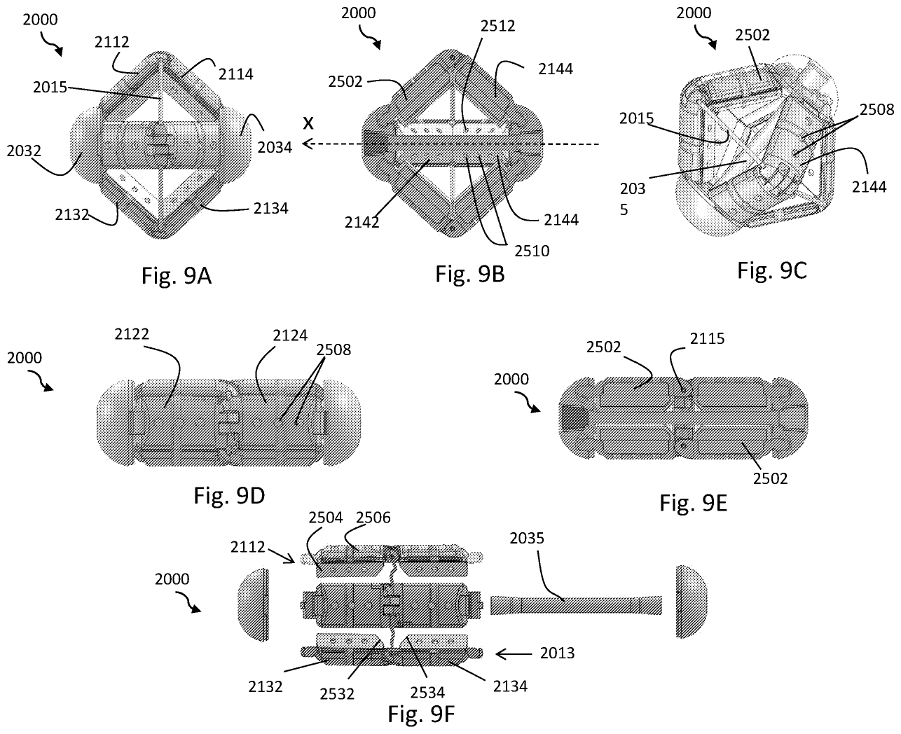

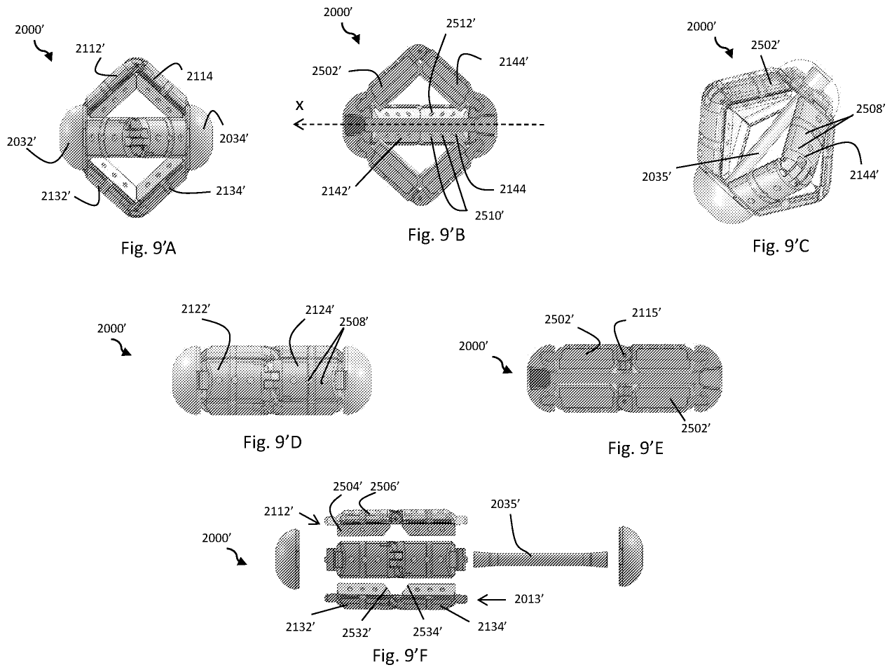

FIGS. 9A-9C, FIGS. 9D-9E, and FIG. 9F show another example of a device 2000 for temporary stomach retention according to the present disclosure, respectively in an expanded (open) configuration (FIGS. 9A-9C), in a closed (swallowing) configuration (FIGS. 9D-9E), and in a disassembled configuration which may form an emptying configuration and/or a pre-assembling configuration during manufacturing (FIG. 9F). FIGS. 9A, 9D, and 9F show a front view of the device 2000. FIG. 9C shows a perspective view of the device 2000 in the same configuration as in FIG. 9A. FIGS. 9B and 9E show a longitudinal cross-section view (along axis X) of the device 2000 in the same configuration respectively as in FIGS. 9A and 9D, the cross-section being taken in a median plane of the device 2000 parallel to the view plane of respectively FIGS. 9A and 9D.

The device 2000 resembles the device 1000 discussed above in many aspects. Accordingly, numerals used to identify features of the device 1000 are incremented by a factor 1000 to identify like features of the device 2000. It is understood that the features which are similar to devices 100 and 1000 and combinable with device 2000 are not all repeated in details below.

The device 2000 may be deformable from the closed configuration of FIGS. 9D-9E into the expanded configuration of FIGS. 9A-9C, and the device 2000 may be irreversibly disassembled from the expanded configuration of FIGS. 9A-9C into an emptying configuration identical or similar to the disassembled configuration of FIG. 9F.

The device 2000 differs from the device 1000 mainly in that in the device 2000, at least one (e.g., all eight) of the arm sections 2112, 2114, 2122, 2124, 2132, 2134, 2142, 2144 comprises an exposed cavity (no numeral reference on the figures).

The cavity allows embedding any desired product inside the arm section. As the cavity is exposed, the embedded product is in fluid communication with the surrounding environment of the device 2000, notably when the device 2000 is in the expanded configuration. In examples, rigid arm sections may easily comprise such cavities thanks to their rigidity. In specific, because of said rigidity, the presence of the cavities does not prevent from having a sufficient structural rigidity of the device 2000, such that it better remains functional and open in the expanded configuration so as to be retained in the stomach.

In particular, the cavity may contain an active pharmaceutical ingredient (API). The API may be provided within a solid form 2502 such as a tablet, which may optionally further contain one or more pharmaceutically acceptable excipients. The solid form 2502 may comprise a therapeutic payload of the API for local release and/or topical treatment of a patient condition at the gastric region or for systemic absorption. The API may be any one of the APIs mentioned earlier, and/or for the treatment of any disease or medical condition mentioned earlier. The solid form 2502 may comprise no protective cover (e.g., a coating), as the device 2000 may comprise a locking container for delivery in the stomach, thereby ensuring that the solid form 2502 remains functional (and optionally substantially intact) until it reaches the stomach region. Alternatively, the solid 2502 may have a coating and/or the exposure apertures 2508, 2510, 2512 (discussed hereinbelow) in the cavity may be coated, so as to expose the inner solid form 2502 only when desired. In variations, the API may be carried by the device in a semi-solid, powder, gel, or liquid form/texture, or in (any combination of) a solid, semi-solid, powder, gel, and/or liquid form (i.e., partly in one form, and partly in one or more other forms).

At least one (e.g., all eight) of the arm sections 2112, 2114, 2122, 2124, 2132, 2134, 2142, 2144 may comprise two components (e.g., recipient or body component 2504 and a cover component 2506) attached one to another and forming the exposed cavity therebetween. Numeral references are provided only for the arm section 2112 and its two components 2504 and 2506 (see FIG. 9F), for the sake of conciseness. Each such component (e.g., 2504 and 2506) may be rigid, and optionally integrally formed, for example made by injection molding or 3D printing or any other known way, from any material such as plastic, a biocompatible polymer such as cellulose acetate, resin, metal, or ceramic, as discussed with reference to the device 1000. The two components of each pair may be attached in any manner, for example snapped (i.e., clicked) one to another, e.g., after insertion of the solid form (e.g., tablet) 2502 between the two components. This facilitates manufacturing.

The at least one arm section (e.g., 2114) may comprise a peripheral wall. Each component of the at least one arm section (e.g., 2114) may comprise one or more respective walls, and said respective walls may compose the peripheral wall. The peripheral wall may have apertures 2508, 2510, 2512 (i.e., openings or holes) formed thereon, providing exposure of the cavity and thereby drug release in the stomach (see FIGS. 9B and 9C). The apertures 2508, 2510, 2512 connect the cavity with the environment of the device, thus putting the cavity into fluid communication with said environment.

One or more (e.g., all) recipient components (e.g., 2504) may comprise such drug release apertures 2508, and/or one or more (e.g., all) cover components (e.g., 2506) may comprise such drug release apertures 2510, 2512. This provides for drug release on one or both sides of each such arm section, thereby achieving a uniform diffusion of the solid form 2502. Alternatively, the apertures may present a design which provides a predetermined release rate.

As shown for the arm section 2114 (see FIGS. 9B and 9C), the recipient component may comprise one or more (e.g., exactly one) ranges of drug release apertures 2508, formed on an external envelope of the device 2000. Each range of drug release apertures 2508 may be linear, straight, and/or evenly distributed. Alternatively or additionally, the cover component may comprise one or more (e.g., exactly two) ranges of drug release apertures 2510, 2512. Each range of drug release apertures 2510 and/or 2512 may be linear, straight, and/or evenly distributed. Each arm section 2114 may present a generally prism shape with a triangle or trapeze cross-section. The drug release apertures 2508 may be formed on a face of the prism corresponding to the base of the triangle or trapeze (i.e., respective wall of the recipient component), and the drug release apertures 2510, 2512 may be formed each on a respective face of the prism to the sides of the triangle or trapeze (i.e., respective walls of the cover component). This achieves an even more uniform diffusion of the solid form 2502, by ensuring exposure everywhere of the cavities inside the arm sections.

The device 2000 may comprise no support tube surrounding the resiliently deformable member 2035. In turn, the arm sections 2112, 2114, 2122, 2124, 2132, 2134, 2142, 2144 may be dimensioned to encounter each other when the device 2000 reaches the expanded configuration (as best seen on FIG. 9C), thus maintaining a minimal distance between the two coupling heads 2032 and 2034, thereby preventing the device 2000 from expanding beyond the maximal authorized diameter. For example, the arm sections (e.g., 2132, 2134) of each articulated arm (e.g., 2013) may comprise each a sloped surface (e.g., 2532, 2534), and each such pair of sloped surfaces may be configured to abut one onto another, so as to block the relative rotation of the two arm sections of the pair (e.g., 2132 and 2134), beyond a predetermined abutment angle (see FIG. 9F). Thanks to the presence of the cavities (e.g., already embedding an API), the device 2000 may authorize a relatively reduced volume occupancy in the expanded configuration (while allowing sufficient drug loading), thus allowing space occupancy induced by said abutting sloped surfaces (e.g., 2532, 2534). In variations, the device 2000 may comprise a support tube.

The device 2000 may comprise a unique thread 2015 located in a single plane. The thread 2015 may lie on a median plane (not shown) of the device 2000 substantially perpendicular to axis X. The hinge pins 2115 part of the pivot-type coupling between the arm sections may be hollow, and the thread may pass through said hinge pins 2115. In variations, the device 2000 may comprise several threads (e.g., in several planes) and/or a unique thread in several planes. Alternatively, the thread 2015 may be pulled through the hinge holes of the pivot-coupling hinge before the hinge pins are inserted, or threads may be glued to the hinge pins. Alternatively, the thread can serve as both a circumferential belt and a hinge pin The thread may be composed of two sections, including a more flexible sections and a more rigid and less flexible section (optionally also wider, designed to be retained by pressure in the hinges cavities). The thread may be pulled through the hinge hole while the rigid section may be placed and retained into the arm hinge as a pin.

In specific implementations of the device 2000, the device may include drug, placed in the arms. Device preparation and assembly may be according to the earlier-described first specific implementations of the device 1000, with the following difference. Before the arms are hinged together with a nylon pin, a tablet containing an API (a model tablet made by 3D printing VERO is used in experimental 2 discussed later) may be placed in each of the arm body cavities. Then, the arm cover may be clicked on the arm body/recipient to lock the tablet inside. Apertures may be designed in both arm body/recipient and the arm cover to allow drug release from arms.

FIGS. 9′A-9′C, FIGS. 9′D-9′E, and FIG. 9′F show another example of a device 2000′ for temporary stomach retention according to the present disclosure, respectively in an expanded (open) configuration (FIGS. 9′A-9′C), in a closed (swallowing) configuration (FIGS. 9′D-9′E), and in a disassembled configuration which may form an emptying configuration and/or a pre-assembling configuration during manufacturing (FIG. 9′F). FIGS. 9′A, 9′D, and 9′F show a front view of the device 2000′. FIG. 9′C shows a perspective view of the device 2000 in the same configuration as in FIG. 9′A. FIGS. 9′B and 9′E show a longitudinal cross-section view (along axis X) of the device 2000′ in the same configuration respectively as in FIGS. 9′A and 9′D, the cross-section being taken in a median plane of the device 2000′ parallel to the view plane of respectively FIGS. 9′A and 9′D.

The device 2000′ resembles the device 2000 discussed above in many aspects. Accordingly, numerals used to identify features of the device 2000 are appended a single quotation mark ′ to identify like features of the device 2000′. It is understood that the features which are similar to device 2000 and combinable with device 2000′ are not all repeated in details below.

The device 2000′ mainly differs from the device 2000 in that it comprises no circumferential thread 2015. The device 2000′ may allow particularly non-obstructed chyme flow, thus allowing long but safe retention of the device at the stomach. Thus, the device 2000′ may be configured for self-emptying from the stomach after a predetermined period of time in safety. Yet, the device may be sufficiently rigid even without any circumferential thread or belt.

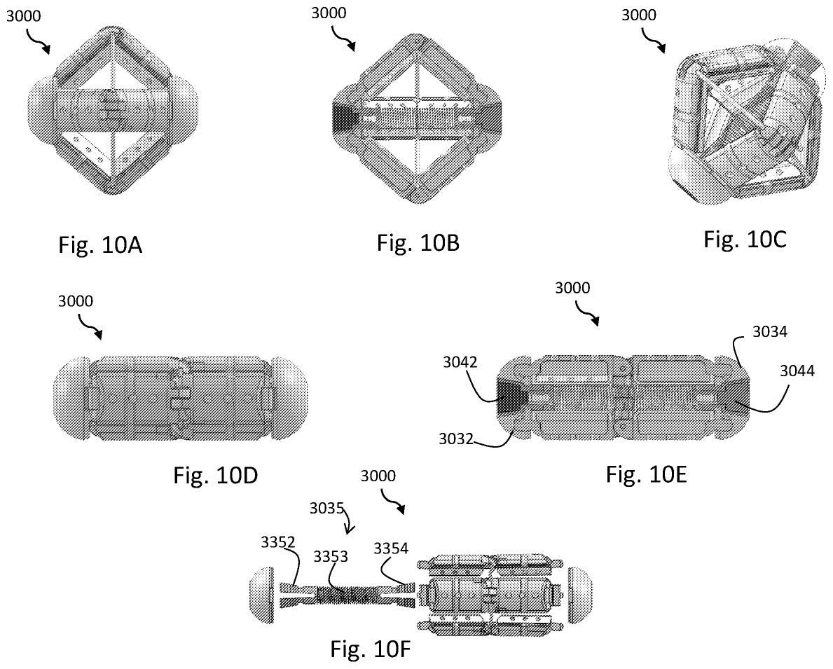

FIGS. 10A-10C, FIGS. 10D-10E, and FIG. 10F show another example of a device 3000 for temporary stomach retention according to the present disclosure, respectively in an expanded (open) configuration (FIGS. 10A-10C), in a closed (swallowing) configuration (FIGS. 10D-10E), and in a disassembled configuration which may form an emptying configuration and/or a pre-assembling configuration during manufacturing (FIG. 10F). FIGS. 10A, 10D, and 10F show a front view of the device 3000. FIG. 10C shows a perspective view of the device 3000 in the same configuration as in FIG. 10A. FIGS. 10B and 10E show a longitudinal cross-section view (along axis X) of the device 3000 in the same configuration respectively as in FIGS. 10A and 10D, the cross-section being taken in a median plane of the device 3000 parallel to the view plane of respectively FIGS. 10A and 10D.

The device 3000 resembles the device 2000 discussed above in many aspects. Accordingly, numerals used to identify features of the device 1000 are incremented by a factor 2000 to identify like features of the device 3000. It is understood that the features which are similar to the devices 100, 1000, and 2000 and combinable with device 3000 are not all repeated in details below.

The device 2000 may be deformable from the closed configuration of FIGS. 9D-9E into the expanded configuration of FIGS. 9A-9C, and the device 2000 may be irreversibly disassembled from the expanded configuration of FIGS. 9A-9C into an emptying configuration identical or similar to the disassembled configuration of FIG. 9F.

The device 3000 differs from the device 2000 mainly in that in the device 3000, the resiliently deformable member is a spring 3035 configured to be stretched (e.g., traction spring), for example made of metal (such as stainless steel or Nitinol). A metal spring has little creep (low compression set) and at the same time a relatively high tension modulus (spring constant). This allows achieving a particularly high emptying force.

In embodiments, the spring 3035 allows achieving an emptying force higher than 500 gr (examples table 2). This may allow particularly long shelf life. Still, as the spring is further stretched when device is transit from open to closed configuration, the tension obtained during closed configuration may be substantially higher (e.g., ≥1000 gr).

The spring 3035 may comprise hinges 3352 and 3354 at its two ends (see FIG. 10F). The hinges 3352 and 3354 may be integrally formed with the stretchable central portion 3353 of the spring 3035. Once the timer 3042 and/or 3044 is eroded, the respective integral hinge 3352 and/or 3354 collapses, enabling the spring 3035 to detach from the respective coupling head 3032 and/or 3034 and disassembly to occur (see FIG. 10E).

In specific implementations of the device 3000, the device may be prepared as the device 2000, only replacing the elastic member by a stainless steel or Nitinol or the like having hinges as described above.

FIGS. 11A-11C, and FIGS. 11D-11E show another example of a device 1000″ for temporary stomach retention according to the present disclosure, respectively in an expanded (open) configuration (FIGS. 11A-11C), and in a closed (swallowing) configuration (FIGS. 11D-11E). FIGS. 11A and 11D show a front view of the device 1000″. FIG. 11C shows a perspective view of the device 1000″ in the same configuration as in FIG. 11A. FIG. 11B shows a longitudinal cross-section view (along axis X) of the device 1000″ in the same configuration respectively as in FIGS. 11A and 11D, the cross-section being taken in a median plane of the device 1000″ parallel to the view plane of respectively FIGS. 11A and 11D.

The device 1000″ resembles the devices 1000 and 1000′ discussed above in many aspects. Accordingly, numerals used to identify features of the device 1000′ are appended a single quotation mark ′ to identify like features of the device 1000″, and similarly, numerals used to identify features of the device 1000 are appended a double quotation mark ″ to identify like features of the device 1000″. It is understood that the features which are similar to device 1000′ and/or 1000 and combinable with device 1000″ are not all repeated in details below.

Referring additionally to FIGS. 11F-11H, the device 1000″ differs from the device 1000 and 1000′ mainly in that it does comprise a support tube 1038″ in which the resiliently deformable member 1035″ is arranged (see FIGS. 11B and 11G), but the support tube 1038″ is different from the support tubes 1038 and 1038′ of the devices 1000 and 1000′.

The support tube 1038″ has similarities with the support tubes 1038 and 1038′, and in particular may be made of any material, such as a rigid material (e.g., plastic, metal, ceramic) or an elastic material. Also, the support tube 1038″ may function as the support tubes 1038 and 1038′. However, instead of apertures 1510 or 1510′ the support tube 1038″ has one or more (e.g., two) peripheral grooves 1382″ (see FIG. 11F), each forming an exposed recess. Each exposed recess allows embedding any desired product. As the cavity is exposed, the embedded product is in fluid communication with the surrounding environment of the device 1000″, notably when the device 1000″ is in the expanded configuration.

In particular, the peripheral grooves 1382″ may each lodge a ring-shaped form 1502″ (see FIG. 11G) containing an API. The forms 1502″ may be solid and/or further contain one or more pharmaceutically acceptable excipients. The forms 1502″ may comprise a therapeutic payload of the API for local release and/or topical treatment of a patient condition at the gastric region or for systemic absorption. The API may be any one of the APIs mentioned earlier, and/or for the treatment of any disease or medical condition mentioned earlier. The forms 1502″ may comprise no protective cover (e.g., a coating), as the device 1000″ may comprise a locking container for delivery in the stomach, thereby ensuring that the form 1502″ remains functional (and optionally substantially intact) until it reaches the stomach region. Alternatively, the form 1502″ may have a coating, so as to release the API only when desired. In variations, the API may be carried by the device in a semi-solid or gel form, or in (any combination of) a solid, semi-solid, powder, gel, and/or liquid form (i.e., partly in one form, and partly in one or more other forms).

The support tube 1038″ may present a narrowed central section 1384″ (see FIGS. 11F and 11H), to provide compactness to the device 1038″, while the extremities of the support tube 1038″ may have a larger diameter so that the peripheral grooves 1382″ presents enough space to lodge the ring-shaped API. In turn, the articulated arms 1011″-1014″ may comprise inner recesses 1380″ (see FIG. 11B) to receive the extremities of the support tube 1038″ (as best seen on FIG. 11E). These inner recesses 1380″ (relative to the periphery of the device), allow compactness of the device 1000″. The rigidity of the arms facilitates the constructions of the inner recesses 1380″, with reduced risks in terms of structural rigidity loss.

In addition, the device 1000′ further differs from the device 1000 in that, like the device 1000′, it comprises no circumferential thread 1015.

In the above-discussed examples of the device, the device is configured to transfer from the expanded configuration into the emptying configuration upon the resiliently deformable member stopping to force the coupling heads together (e.g., due to release of the resiliently deformable member from one or both the coupling heads), for example when the device in the expanded configuration is exposed to a predetermined activation signal. In variations, the device may be configured to transfer from the expanded configuration into the emptying configuration upon the coupling heads stopping to compress and bend the arms, for example due to release of the coupling between the arms and the coupling heads. In examples, the coupling between the arms and the coupling heads may integrate one or more support elements constituting fixations between the arms and the coupling heads, while having triggering capabilities as described in the examples, and notably configured to degrade (e.g., when exposed to the predetermined activation signal) and thereby disassemble the arms from the coupling heads, while optionally the resiliently deformable member stays assembled to both the couplings heads (and may continue to force the coupling heads together).

In the above-discussed examples of the device, the resiliently deformable member (e.g., elastic or spring) is arranged between the arms. In variations, the resiliently deformable member may be arranged elsewhere. For example, the resiliently deformable member may be arranged on the sides of the arms, or inside the arms. In yet another example, the resiliently deformable member may form an arm of the device itself.

In the above-discussed examples of the device, the support elements (e.g., timers) are located inside the coupling heads, in particular in a hollow portion thereof. In variations, one or more support elements may be located elsewhere. For example, a resiliently deformable member (e.g., elastic or spring) may be made of two separate (i.e., initially disconnected) halves, the two halves being connected by a support element. When the support element degrades, the two halves disconnect, thereby the resiliently deformable member stops forcing the two coupling heads together and thus triggers transfer into the emptying configuration.

In the above-discussed examples of the device, the arms are each arranged longitudinally (on axis X) alongside each other. In variations, one or more of the arms may be arranged differently. For example, the arms may be twisted together.

In the above-discussed examples, the load of API may be distinct and separate from the support elements (e.g., timers). Each support element may be integrally formed, and the load of the API may be elsewhere. Notably, the support elements are located inside the coupling heads, while the discussed load of the API is located between the coupling heads and the arms. The support element may comprise none of the API.

The above-discussed device may be adapted for a method of treating a condition which benefits from local dispensing of an API in the gastric region of a subject suffering from said condition, comprising administering to said subject the device.

FIG. 12 illustrates retention testing equipment 200 that can be used in a method for assessing the capability of a device to pass through the pyloric valve using retention testing equipment. Basically, a method for assessing the capability of a device to pass through the pyloric valve comprises: providing a retention test device configured to simulate the geometry of a standard pyloric valve region; placing a device in the retention test device upstream of a simulated pyloric valve; applying sequentially on the device increasing forces (e.g., substantially constant forces), each forces being preferably applied for a predetermined test time period (e.g., about 10 seconds), wherein said forces tend to pull the device out of the simulated pyloric valve, until an extraction force which is sufficient for extracting the device from the simulated pyloric valve within the test time period is reached; comparing the extraction force to a predetermined threshold (e.g., about 1.5 N) defining a minimal retention capability.

The retention test equipment 200 may include a retention test device 210 (also referred to as simulated pyloric valve) and a mounting support 250 for maintaining the retention test equipment vertically so that gravity tends to extract the device 100 through the simulated pyloric valve.

The retention test device 210 for simulating the geometry of standard pyloric valve region may generally include a funnel portion reproducing the narrowing of the pyloric valve. In more details, the test device 210 may comprise a first tubing portion 211 of a first diameter D1 joined at its connecting end to a narrowing truncated conical portion 212. An end of the truncated conical portion 212 opposed to the connecting end may be of a second diameter D2 smaller than D1. The narrowing conical portion 212 may be concentric to a second tubing portion 213 surrounding the narrowing conical portion 212. The second tubing portion 213 may be joined to the connecting end of the first tubing portion 211. In some embodiments, as illustrated, the first and second tubing portions 211 and 213 may be of same diameter D1 and form a single tube. In some embodiments, the inner surface of the retention test device may be lubricated, for example with an edible oil, for example corn oil. In the following, the first tubing portion 211 and the narrowing portion 212 are also referred together as donor chamber and the second tubing portion 213 is also referred to as acceptor chamber. The first tubing portion 211 may be configured to simulate the stomach, more particularly the pyloric region of the stomach while the second tubing portion 213 to simulate the duodenum. The first diameter D1 of the first tubing portion 211 may be of about 20 to 35 mm, preferably of about 30 mm. A length of the first tubing portion 211 may be of about 100 mm. The conical narrowing portion 212 may be configured to simulate the stomach geometry. A length of the narrowing conical portion may be of about 12 mm. The second diameter D2 may be of about 10 to 20 mm, preferably of 16 mm. A length of the second tubing portion 213 may be of about 70 mm. The retention test device 210 may be made out of a rigid material.

The device 100 may be positioned at the connecting end of the first tubing portion 211 i.e., at the enlarged part of the funnel. The device 100 may be wrapped into a foil 230 on which increasing weights may be attached so as to pull the device out of the donor chamber into the acceptor chamber. The foil 230 may be preferably made of low density polyethylene with a thickness of about 10 microns. The foil may preferably have a rectangular shape of about 19 cm by 25 cm. An external surface of the foil 230 in contact with the test device 210 may be lubricated, preferably with corn oil.

The foil 230 may be sequentially attached to increasing weights in order to apply increasing forces on the device. The weights may be between about 10 gr to 500 gr. A predetermined extraction force threshold may correspond to a predetermined extraction weight threshold. The predetermined extraction weight threshold defining a (minimal) satisfying retention capability may be of about 150 gr, 250 gr, 300 gr or 350 gr, 400 gr, 450 gr, 500 gr or 600 gr.

In some embodiments, the previously described method may be repeated several times and an average extraction force needed to extract the device out of the simulated pyloric valve may be determined.

The previously described method may advantageously be used to determine the capability of a device in the expanded configuration to be retained in the stomach, i.e., to not pass through the pyloric valve. In these embodiments, the device in the expanded configuration is considered to satisfy the retention capability if the measured extraction force is above the predetermined minimal retention threshold. In some embodiments, the method previously described may also be used to determine the capability of a device in the emptying configuration to be emptied i.e., to pass through the pyloric valve. In these embodiments, the comparing step may instead be: comparing the extraction force to a predetermined threshold defining a maximum acceptable retention capability. In these embodiments, the device in the emptying configuration is considered to satisfy the emptying capability if the measured extraction force is below the predetermined maximal retention threshold.

In particular, a method for determining the retention capability of a device includes: providing a retention test device mounted vertically, wherein the retention device includes a first tubing portion of a first diameter of about 30 mm joined at its connecting end to a narrowing truncated conical portion, an end of the truncated conical portion opposed to the connecting end being of a second diameter of about 16 mm, a length of the first tubing portion being of about 100 mm, a length of the narrowing truncated conical portion being of about 12 mm, the first tubing portion and narrowing truncated conical portion may be of rigid plastic; placing a device on the narrowing truncated conical portion; applying sequentially on the device increasing forces (e.g., substantially constant forces), each forces being preferably applied for about 10 seconds, wherein said forces tend to pull the device out of the narrowing truncated conical portion, until an extraction force which is sufficient for extracting the device from the narrowing truncated conical portion within 10 seconds is reached; comparing the extraction force to a predetermined threshold of about 1.5 N or 2.0 N.

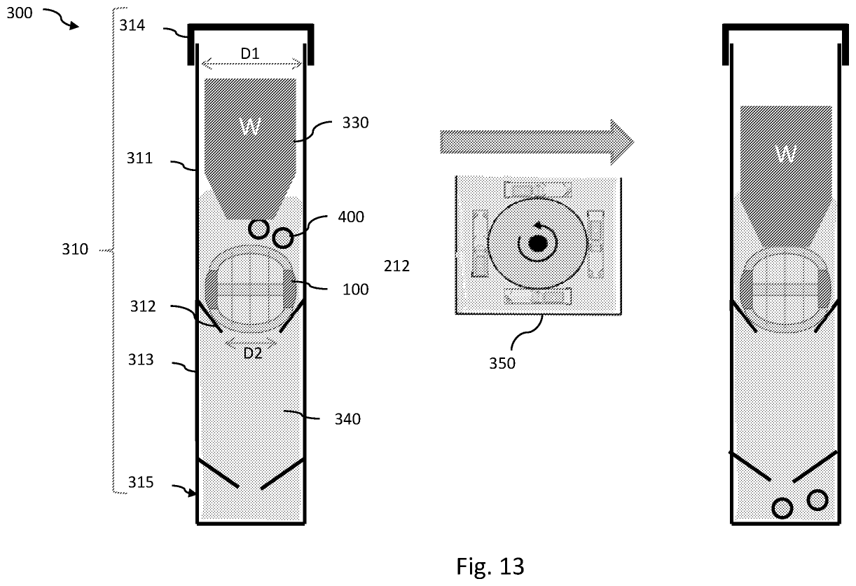

FIG. 13 shows a blocking test equipment 300 useful in a method for assessing the optional capability of a device, while being stopped (i.e., blocked) by the pyloric valve, to allow chyme flow when the device is at the pyloric valve.

Basically, a method for assessing the optional capability of a device, while being stopped by the pyloric valve, to allow chyme flow may comprise: providing a blocking test device configured to simulate the geometry of a standard pyloric region; placing the device in the blocking test device at a position upstream of a simulated pyloric valve; placing at least one chyme representative object configured to simulate standard maximal chyme elements at the pyloric region upstream of the device; exposing the blocking test device to simulated standard GI motility conditions for a predetermined blocking test time period; determining how much of the at least one chyme imitator object have passed through the simulated pyloric valve after the predetermined blocking test period.

The blocking test equipment 300 may include a blocking test device 310 and a rotating system 350.

The blocking test device 310 resembles the retention test device 210 discussed above in that it is configured to simulate the geometry of a pyloric valve. Accordingly, numerals used to identify features of the device 100 are incremented by a factor 100 to identify like features of the retention test device 210.

As previously described with reference to the simulated pyloric valve in the retention test device 210, the blocking test device 310 includes a simulated pyloric valve comprising: a first tubing portion 311, a narrowing truncated conical portion 312 and a second tubing portion 313. Additionally, the blocking test device 310 includes a proximal cover 314 for sealing an upstream end of the first tubing portion (i.e., an end opposed to the connecting end of the first tubing portion 311) and a distal cover 315 for sealing a downstream end of the second tubing portion (i.e., an end opposed to the end of second tubing portion 312 connected to the first tubing portion 311). The distal cover 315 may include a valve mechanism to allow objects passing through the narrowing portion 212 to be captured at the bottom of the acceptor chamber. A length of the distal cover may be of about 14 mm.

The device 100 may be positioned at the connecting end of the first tubing portion 311 i.e., resting upwards of the enlarged part of the funnel.

In order to test the capability of a device to allow chyme flow, at least a chyme representative object 400 may be placed upstream of the device 100.

The at least one chyme representative object 400 may be spherical beads of a diameter of about 6 mm or less. Such size may be representative of standard maximal chyme elements at the pyloric region. It is understood that such standard maximal chyme element size is referring to standard ingested food i.e., provided that the subject does not ingest food with large non-edible elements e.g., fruit pits from dates, plums, apricot, etc.

The rotating system 350 may enable to simulate GI motility. For example, the rotating system may enable to rotate the trapping test equipment 300 together with the device 100 and the at least one chyme representative object 400 at a rate of between about 2 to 20 rpm. A diameter of the rotation movement may be between about 250 mm to 350 mm. Additionally, in order to simulate standard GI motility conditions, a weight 330 may be placed upstream of the device 100 so as to push the device through the simulated pyloric valve (donor chamber). The weight may be of about 30 to 100 g. The weight may have a first cylindrical portion prolonged by a second narrowing truncated conical portion intended to be in contact with the device 100 and to enable the weight to push the device into the second tubing portion 313. The first cylindrical portion may have a diameter of about 30 mm and a length of about 75 mm to 100 mm, more preferably 85 mm. The second truncated conical portion may be 10 mm long and a diameter at the end of the truncated conical portion may be of about 14 mm. Additionally, in order to simulate standard human GI motility conditions, a solution of such as hydrochloric acid (HCl) 0.05M pH 1.3 or HCl 0.01M, pH 2 or such may be added in the test device 310. Additionally, the temperature of the solution may be maintained at about 37° C. For example, the rotating system 350 may include a bath at 37° C. in which the rotation is performed.

Similarly, a ratio between the amount of chyme representative objects having passed through the simulated pyloric valve (i.e., captured by the distal cover 315) and the amount of chyme representative objects placed initially upstream of the device can be determined. The ratio can be compared with a predetermined threshold representative of a minimal expected trapping success rate.

In particular, a method for testing the capability of a device, while being stopped by the pyloric valve, to allow chyme flow includes:
providing a blocking test device;
wherein the blocking test device includes a first tubing portion of a first diameter of about 30 mm joined at its connecting end to a narrowing truncated conical portion, an end of the truncated conical portion opposed to the connecting end being of a second diameter of about 16 mm, a length of the first tubing portion being of about 100 mm, a length of the narrowing truncated conical portion being of about 12 mm, the first tubing portion being further connected at its connecting end to a second tubing portion of diameter 30 mm, the second tubing portion being 96 mm long and comprising a distal cover sealing a distal end of the second tubing portion, the distal cover including valve mechanism to allow objects passing through the narrowing portion to be captured at a distal end of the second tubing portion, the first tubing portion comprising a proximal cover sealing the proximal end of the first tubing portion, the first tubing portion, second tubing portion and narrowing truncated conical portion being made of rigid plastic;
placing the device in the blocking test device over the narrowing truncated conical portion;
placing at least one (e.g., 3) glass spherical bead of about 6 mm diameter upstream of the device;
adding a solution of HCl 0.01M, pH 2 at 37° C. in the blocking test device;
placing a weight of 100 g upstream of the device;
rotating the blocking test device and its content at 5 rpm for 30 minutes in a bath maintained at 37° C.;
determining how many of the glass spherical beads have passed through the simulated pyloric valve after the 30 minutes period.

This invention will be better understood by reference to the experimental details, which follow, but those skilled in the art will readily appreciate that the specific experiments detailed are only illustrative.

### Experimental 1

In vitro testing of a device according to the present disclosure was performed.

Test results obtained with a device having a structure as shown in FIG. 1 and manufactured according to the assembly method detailed hereinabove (four arm device with support elements as Eudragit® L or S pins i.e., degradable at pH above 6 and 7, respectively) is now described. The device had folded dimensions of 10.5 mm in diameter and 29 mm in length.

### Shelf Life Test

The device was subject to shelf life tests. The device was placed in the closed configuration within a capsule shape container of 30 mm height by 11 mm diameter. After respectively 1 day, 7 days, the device was extracted from the container and subject to the test described with reference to FIG. 13 to determine if it maintained its properties of allowing passage of 6 mm spherical glass beads (representative of standard maximal chyme elements present in the stomach). The device was also subject to the test described with reference to FIG. 12 to determine if it maintained its property of not passing through the pyloric valve in the expanded configuration.

The results are listed in table 1 below:

TABLE 1
Shelf life test results

| | % of 6 mm glass | |
|---|---|---|
| Days | beads blocked * | Emptying force (D2 = 16 mm) |
| 0 | 0% | 1.5N |
| 1 | 0% | 1.5N |
| 7 | 0% | 1.5N |

* 2 beads tested 4 times

### Experimental 2

### 1. In Vitro Testing of Devices 1000, 2000 and 3000

### Device 1000

A device was prepared in accordance with the implementations of device 1000. The assembled device was then subjected to the two in vitro retention (emptying) tests, as described in reference to FIGS. 12-13 (with D1=30 mm, and D2=16 mm or 18 mm). Test results were acceptable. 0% of the 6 mm glass beads were retained, the emptying force was 3.5N (D2=16) and 2.5N mm (D2=18 mm). Results are summarized in table 2, below.

### Device 2000

A device was prepared in accordance with the implementations of device 2000. The assembled device was then subjected to the two in vitro retention (emptying) tests, as described in reference to FIGS. 12-13 (with D1=30 mm, and D2=16 mm or 18 mm). Test results were acceptable. 0% of the 6 mm glass beads were retained, the emptying force was 3.5.5N (D2=16 mm) and about 2.5N (D2=18 mm). Results are summarized in table 2 below.

### Device 3000

A device was prepared in accordance with the implementations of device 3000. The assembled device was then subjected to two in vitro retention test, as described in reference to FIGS. 12-13 (D1=30 mm, D2=16 mm or 18 mm). Test results where acceptable. 0% of the 6 mm glass beads were retained, the emptying force was >5N (D2=16 mm) and about 5N (D2=18 mm). Results are summarized in table 2 below.

### Device 2000′

A device was prepared in accordance with the implementations of device 2000′. The assembled device was then subjected to the two in vitro retention (emptying) tests, as described in reference to FIGS. 12-13 (with D1=30 mm, and D2=16 mm or 18 mm). Test results were acceptable. 0% of the 6 mm glass beads were retained, the emptying force was 3.5N (D2=16) and 2.5N mm (D2=18 mm). Results are summarized in table 2 below.

### SUMMARY

The rigid structure of the tested devices enables significant achievements. While maintaining the transition of 6 mm beads, it achieves higher device retention (in-vitro emptying force test), higher volume void enabling drug loading in device and small capsule size, enabling easy swallowing.

TABLE 2
results of in-vitro testing

| | | folded | | | | | % of 6 mm |
|---|---|---|---|---|---|---|---|
| | | dimension | unfolded | potential | emptying | emptying | glass |
| | biasing | (Ø, length | dimensions | capsule | force (grF)* | force (grF)* | beads |
| device | system | in mm) | (Ø in mm) | dimension | (D2 = 16 mm) | (D2 = 18 mm) | blocked |
| 1000 | silicone | 9.8, 28.8 | 24 | 10.2, 29 | 350 | 250 | 0% |
| 2000 | silicone | 9.8, 28.8 | 24 | 10.2, 29 | 350 | 250 | 0% |
| 3000 | stainless | 9.8, 28.8 | 24 | 10.2, 29 | >500 | >500 | 0% |
| | steel | | | | | | |
| | spring | | | | | | |
| 4000 | stainless | 10.2, 29 | 24.5 | 10.5, 29.5 | >500 | >500 | 0% |
| | steel | | | | | | |
| | spring | | | | | | |
| 2000′ | silicone | 9.8, 28.8 | 24 | 10.2, 29 | 350 | 250 | 0% |

*emptying test applied right after assembly, based on FIGS. 12-13 D1 = 30 mm

### 2. Pre-Clinical In Vivo Study

### Rigid Device Preparation for Pre-Clinical In Vivo Study

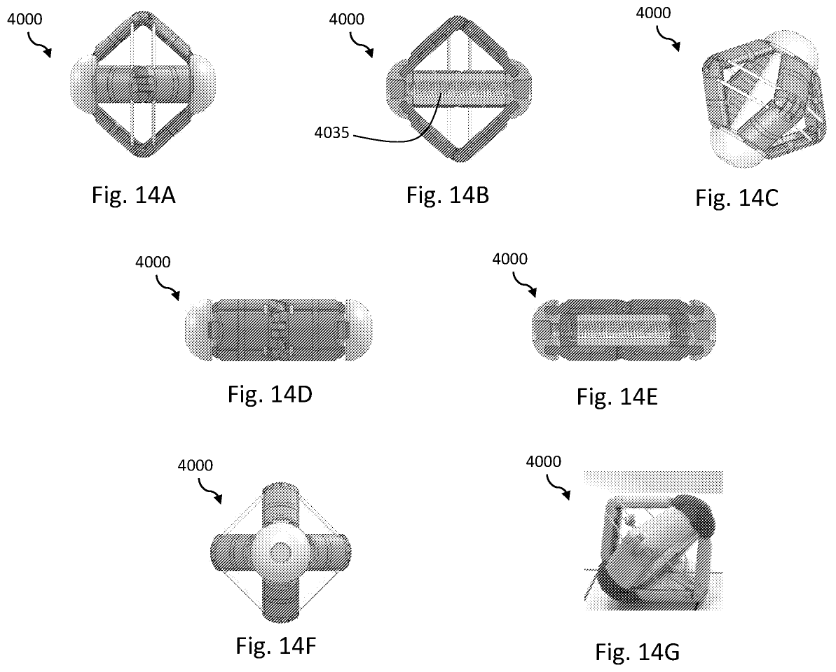

With reference to FIGS. 14A-14G, in order to test performance of the device in vivo, a rigid device 4000 based on the principles of devices 1000, 2000 and 3000, adapted for administration to pig was prepared. FIGS. 14A-14C and FIGS. 14D-14E show schematically the device 4000 for temporary stomach retention in a pig, respectively in an expanded (open) configuration (FIGS. 14A-14C) and in a closed (swallowing) configuration (FIGS. 14D-14E). FIG. 14F shows a top view of the device 4000 and FIG. 14G shows a photograph of the prepared device.

Device preparation and assembly was done as follows: device arms and head were made by Stratasys 3D printer rigid VERO material. Each of the arms was labeled with a barium thread to enable identification in X-ray imaging. A dummy 3D tablet was placed in each arm. Each two arms were hinged together with a nylon pin. A stainless steel biasing spring 4035 of 1.9 mm diameter was pulled through one of the heads. The first end was locked by a stainless steel locking element. The second end was pulled through the second head, and locked by a second locking element. Arms were then hinged to the heads via a designated arm-head locking structure. Device dimensions in the folded configuration were about 29 mm in length and 10.2 mm in diameter, while in the unfolded configuration the diameter was of about 23.5 mm.

The assembled device was then subjected to an in vitro retention test, as described in reference to FIGS. 12-13 (D1=30 mm, D2=16 mm). Test results were acceptable. 0% of the 6 mm glass beads were retained, the emptying force was 5N (D2=16 mm). Results are summarized in table 2 and discussed above.

### Dosing System Preparation for Pre-Clinical In Vivo Study

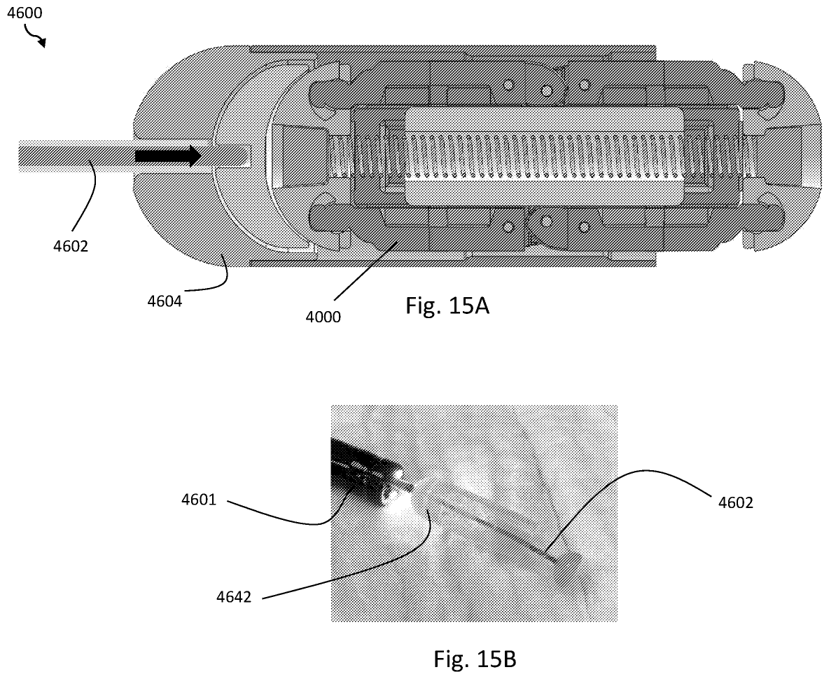

With reference to FIGS. 15A-15B, in order to dose the device into a pig's GI tract via a gastroscope 4601, a dosing system 4600 was prepared.

FIG. 15A shows schematically the dosing system, and FIG. 15B shows a photograph of the dosing system. A housing adaptor 4604 was made by a Stratasys 3D printer. The dosing adaptor was designed to house the device in the folded configuration in one end, and to be connected to an ejection lead 4602 (US endoscopy 00711144) on the other hand. The assembly of the dosing system was made as follows: the ejection lead 4602 was first pulled through the gastroscope 4601, the housing adaptor 4604 was then connected to the ejection lead 4602 and then, the device 4000 was placed in the housing adaptor 4604 so to enable ejection of the desired site in the animal GI.

### Platforms Tested

The study was aimed to evaluate the performance of the GRDF system in an animal model. The tested performance features included administration, unfolding of the device in stomach, retention of the device in stomach. Deliberately, no emptying timer mechanism was tested (instead of timers in the device 4000, support members of non-dissolving rigid material were fixed).

The trapping device 4000 as well as the control tablet were labeled with barium to enable detection by X-ray and CT scan imaging to assess transit along the GI. Control tablet was not erodible and had a conventional size tablet was made of 20% barium and 79% of ethyl cellulose and 1% magnesium stearate. Tablet was compressed in conventional tableting machine and then coated with 6% cellulose acetate solution in acetone. The coat was left to dry. Tablet remained intact when placed in water for 48 hr. Features are provided in table 3 below:

TABLE 3
Features of the tested platform

| feature | platform tested |
|---|---|
| device | 4000 |
| arms and head | 3D printing (Vero material) |
| inner tablet | 3D printing (Vero material) |
| biasing member | stainless steel |
| expanded diameter (mm) | 23.5 |
| expanded minimal circumference | 70 |
| (mm) | |
| folded length/diameter | 29/10.2 |
| timer pin material | stainless steel |
| emptying test force (gr) | 500 |
| (D2 = 16 mm) | |
| control tablet | non erodible, labeled with barium |
| tablet length/diameter (mm) | 24.5/9 |

Animals: 2 domestic pigs, each weighing 40-45 kg were acclimated for seven days before dosing. The animals were weighed and food consumption was assessed.

Study procedure: (dosing and monitoring) were performed as described below

12 hr prior to dosing, animals were fasted. On day 1 (t=0) animals were anesthetized and dosing was conducted. Dosing was conducted with the aid of a dosing system described in disclosure and a gastroscope.

First pig was dosed one device in the stomach. Second pig was dosed two devices and after seven days also dosed a tablet into the stomach. Once every day (day 1-11) X-ray was conducted to locate objects dosed. On day 4 and 5, in addition to X-ray, a CT imaging was conducted. Following dosing and every day after animals were given a daily conventional meal while water was ad libitum. At day 11, animals were euthanized and autopsy was done.

Study protocol is summarized in table 4.

TABLE 4
in vivo study protocol

| day | test |
|---|---|
| 1 | First and second pig device into stomach. X-ray imaging |
| 2-10 | X-ray imaging |
| 4, 5 | X-ray imaging + CT |
| 7 | 2^{nd} pig dosed with a dummy tablet into stomach |
| 11 | X-ray imaging + euthanasia + autopsy |

### Study Success Criteria

The device is retained in the stomach until the end of the study while control tablet is not retained.

No significant safety matters are observed in the animals and general status and gross pathology of the GI appear normal.

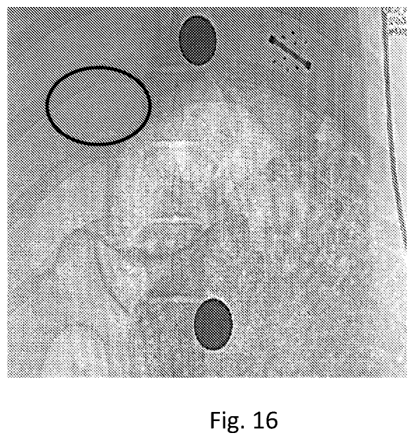

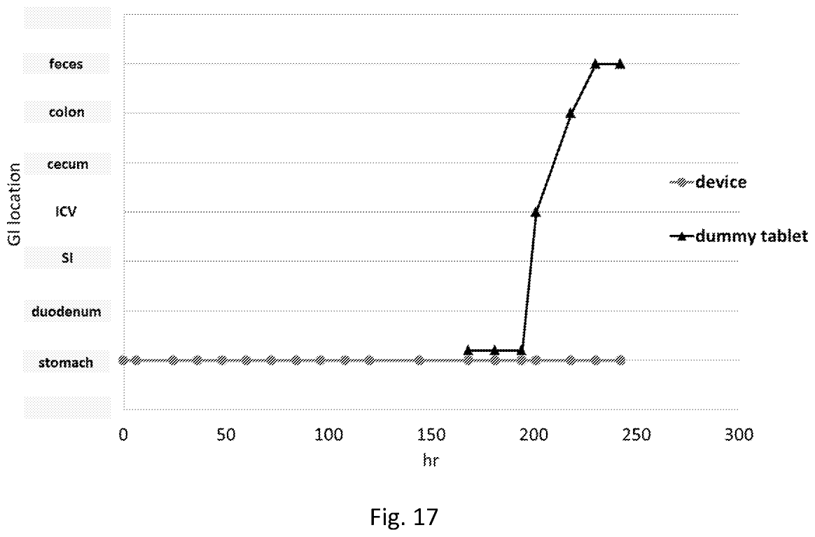

### Study Results (See FIGS. 16-17):

Retention: in both animals, devices were retained in stomach for 11 days (FIG. 16 an X-ray image of the device in the stomach), while tablet emptied from stomach within 2 days (46 hr) after doses. Results are given in FIG. 16.

Safety: No safe abnormalities observed. Animal food consumption, feces, behavior were normal. GI gross pathology was normal with no stomach abnormalities observed.

### 3. Emptying Test: Device Disassembly —Timer Pin Type a and Type B

Rigid devices according to specific implementations of device 1000 were prepared while the biasing member was made of straight silicone tube 55A shore, 3/1 diameter (without the curved locking corners 1325, 1345 described with reference to FIG. 8E). Timer type A and type B were made according to the specific implementations provided with reference to device 1000. Type A timer made of HPMC AS HG:MG:DBS ratio 8:2:1 that dissolves in pH above 6.5 while type B timer made of Eudragit® EPO that dissolves in pH below 5.

A total of four devices were prepared: two with type A timer and two with type B timer. An emptying test was applied as described in reference to FIGS. 12-13, while the medium used are as described in table 5, below. i.e. from 0-24 hr HLC pH 2, and from 24 hr 5 gr bicarbonate added to obtain a pH of 7. Type A timer was retained unfolded at least 24 hr (in HCl media) and disassembled within <24 hr after exposure to pH 7 while device with type A timer was unfolded at least 3 hr and found disassembled at 4 hr (in HCl media).

TABLE 5
device disassembly test with timer pin types A and B

| | | | Type A timer | |
|---|---|---|---|---|
| | | | HPMC AS | Type B timer |
| hr | medium | pH | HG:MG:DBS | Eudragit ® EPO pin |
| 0-24 | HCl pH 0.01M | 2 | retained | device disassembled |
| | | | unfolded | after 4 hr |
| 24 hr | HCl pH 0.01M + | 7 | device | |
| | 5 gr sodium | | disassembled | |
| | bicarbonate | | after <24 hr | |

The results exemplify two optional timer principles:

Type A: a timer made of at least partially an enteric polymer which is activated by a basifying agent which increases the local gastric pH to erode the pin to induce disassembly.

Type B: a timer made of a gastric soluble material (e.g. Eudragit® EPO). The erosion rate can be controlled by adding a pharmaceutical material (e.g. controlled release polymer) to control the release e.g. to prolong timer erosion duration, either in the timer matrix or as a coat.

All patent documents and publications are herein incorporated by reference to the same extent as if each individual publication was specifically and individually incorporated by reference.

For the foregoing embodiments, each embodiment disclosed herein is contemplated as being applicable to each of the other disclosed embodiments. For instance, the elements recited in the device embodiments can be used in the method or kit embodiments described herein and vice versa. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be contemplated by those skilled in the art, and that such modifications and variations are considered to be within the scope of this invention as defined by the appended claims.

1. A device for temporary residence in a stomach of a subject, the device comprising:
at least two arms, each arm of the at least two arms comprising a first end and a second end;
first and second coupling heads, wherein the first end of each arm is coupled to the first coupling head and the second end of each arm is coupled to the second coupling head;
a resiliently deformable member configured to force the first and second coupling heads together to bend the arms thereby biasing the device in an expanded configuration; and
a load of an active pharmaceutical ingredient,
wherein in the expanded configuration and positioned in the stomach, the device is configured to be retained in the stomach of the subject,
wherein the device is configured to transition from the expanded configuration into an emptying configuration,
wherein in the emptying configuration, the device is configured to pass through the pyloric valve, and
wherein in the expanded configuration and positioned in the stomach, the device is configured to at least partially release the active pharmaceutical ingredient.

2. The device of claim 1, wherein at least one arm of the at least two arms is articulated or comprises a set of arm sections, wherein each arm section of the set of arm sections is rigid.

3. The device of claim 2, wherein at least one arm section of the set of arm sections comprises a rigid material, a semi-rigid material, or a flexible material.

4. The device of claim 2, wherein the set of arm sections of the at least one arm are coupled end to end with a pivot-type coupling, wherein the pivot-type coupling comprises transversal hinge holes and a hinge connector passing through the transversal hinge holes.

5. The device of claim 2, wherein the at least two arms comprise at least three arms,
wherein each arm of the at least three arms comprises two arm sections, one arm section of the two arm sections being coupled at one end to the first or second coupling head and at the other end to the other arm section of the two arm sections,
wherein each arm section is substantially of a same length, and
wherein the device has a generally bipyramidal shape in the expanded configuration.

6. The device of claim 2, wherein at least one arm section of the set of arm sections comprises an exposed cavity that contains at least part of the load of the active pharmaceutical ingredient, wherein the at least one arm section of the set of arm sections comprises a peripheral wall, the peripheral wall defining apertures that expose the exposed cavity.

7. The device of claim 1, further comprising a trigger assembly configured to cause the device to transition from the expanded configuration into the emptying configuration upon activation,
wherein the trigger assembly is configured to activate when the device in the expanded configuration is exposed to an activation signal, and
wherein the activation signal comprises exposure to standard stomach environmental conditions for a predetermined residence time period.

8. The device, of claim 1, wherein the resiliently deformable member is secured to both the first and second coupling heads, and the resiliently deformable member is configured, for each activation signal when the device is in the expanded configuration, to be released from the first or the second coupling head, thereby causing the device to transition from the expanded configuration into the emptying configuration.

9. The device of claim 8, further comprising a trigger assembly configured, when the device is in the expanded configuration and is exposed to the activation signal, to release the resiliently deformable member from the first or the second coupling head, thereby causing the device to transition from the expanded configuration into the emptying configuration, wherein the activation signal comprises exposure to standard stomach environmental conditions for a predetermined residence time period.

10. The device of claim 9, wherein the trigger assembly comprises one or more support elements configured to temporarily secure the resiliently deformable member to the first or the second coupling head, and thereby temporarily maintain the device in the expanded configuration.

11. The device of claim 1, wherein in the expanded configuration, the device defines a convex hull having at least one of a sphericity above 0.8 or a ratio between a maximal circumference and a minimal circumference below 1.5.

12. The device of claim 1, wherein the device:
defines an inner space in a closed configuration formed around the resiliently deformable member between the at least two arms and the first and second coupling heads, the inner space containing at least part of the load of the active pharmaceutical ingredient, the device being configured to be deformable from the closed configuration into the expanded configuration;
comprises at least one portion or component that defines an exposed cavity, the exposed cavity containing at least part of the load of the active pharmaceutical ingredient, wherein the at least one portion or component comprises a peripheral wall, the peripheral wall defining apertures that expose the exposed cavity;
defines an exposed recess, the exposed recess lodging at least part of the load of the active pharmaceutical ingredient; or
comprises a coating on an exposed surface, the coating comprising at least part of the load of the active pharmaceutical ingredient.

13. The device of claim 1, wherein the load of the active pharmaceutical ingredient is in a solid, semi-solid, powder, gel, and/or liquid form.

14. The device of claim 1, wherein the device is deformable from a closed configuration into the expanded configuration.

15. The device of claim 14, wherein the load of the active pharmaceutical ingredient is in an embedded dosage form that occupies at least 5% of a volume of a convex hull defined by the device in the closed configuration.

16. The device of claim 14, wherein the device in the closed configuration is configured to be ingested, and
wherein the device in the closed configuration is configured to fit in a cylinder of at least one of a length of 35 mm or less or a diameter of 12 mm or less.

17. The device of claim 15, further comprising a container to temporarily at least partially enclose the device in the closed configuration,
wherein the container is configured to degrade in stomach environmental conditions.

18. The device of claim 1, wherein the first end of each arm of the at least two arms is releasably coupled to the first coupling head,
wherein the second end of each arm of the at least two arms is releasably coupled to the second coupling head,
wherein, in the expanded configuration, the first and second ends of each arm of the at least two arms are each inserted in a respective cavity of the first and second coupling heads, the resiliently deformable member maintaining the first and second ends of each arm of the at least two arms inside the respective cavity of the first and second coupling heads, and
wherein, when the device transitions from the expanded configuration into the emptying configuration, the first and second ends of each arm of the at least two arms are each configured to move out of the respective cavity to disassemble each arm of the at least two arms from the first and second coupling heads.

19. A system comprising:
a device for temporary residence in a stomach of a subject, the device comprising:
at least two arms, each arm of the at least two arms comprising a first end and a second;
first and second coupling heads, wherein the first end of each arm is coupled to the first coupling head and the second end of each arm is coupled to the second coupling head; and
a resiliently deformable member configured to force the first and second coupling heads together to bend the at least two arms thereby biasing the device in an expanded configuration; and
a load of an active pharmaceutical ingredient, wherein in the expanded configuration and positioned in the stomach, the device is configured to be retained in the stomach of the subject, wherein the device is configured to transition from the expanded configuration into an emptying configuration, and wherein in the emptying configuration, the device is configured to pass through a pyloric valve of the subject; and
a dosage form for administering an emptying agent, the dosage form comprising an effective amount of the emptying agent, wherein the effective amount of the emptying agent is sufficient to cause a stomach environment to reach at least one activation environmental condition thereby causing the device to transition into the emptying configuration.

20. A system comprising:
a device for temporary residence at an ileocecal valve of a subject, the device comprising:
at least two arms, each arm of the at least two arms comprising a first end and a second end;
first and second coupling heads, wherein the first end of each arm is coupled to the first coupling head and the second end of each arm is coupled to the second coupling head; and
a resiliently deformable member configured to force the first and second coupling heads together to bend the arms thereby biasing the device in an expanded configuration, wherein in the expanded configuration and positioned at the ileocecal valve, the device is configured to be retained at the ileocecal valve of the subject while allowing chyme flow, wherein the device being is configured to transition from the expanded configuration into an emptying configuration, and wherein in the emptying configuration, the device is configured to pass through the ileocecal valve; and
a dosage form for administering the dosage form comprising:
(a) an amount of an active pharmaceutical ingredient effective to treat a condition that benefits from local dispensing at the ileocecal valve;
(b) a coating for inhibiting release of the active pharmaceutical ingredient in a gastric environment and enabling release of the active pharmaceutical ingredient in a region of the ileocecal valve; and
(c) external dimensions that enable the dosage form to be blocked by the device while allowing chyme flow at the ileocecal valve of the subject in the expanded configuration and when the dosage form reaches the region of the ileocecal valve.
